SEQUENCE LISTING (40 sequences)

SEQ ID NO: 1 - DNA, 18 bp, Artificial Sequence
  SYNTHESIZED
ggagggggttgaggtgtt

SEQ ID NO: 2 - DNA, 23 bp, Artificial Sequence
  SYNTHESIZED
tgtgcacttttattggtctcaag

SEQ ID NO: 3 - DNA, 23 bp, Artificial Sequence
  SYNTHESIZED
cagtcatgaggatccattacctt

SEQ ID NO: 4 - DNA, 20 bp, Artificial Sequence
  SYNTHESIZED
aatttgggtaaaggctgcaa

SEQ ID NO: 5 - DNA, 21 bp, Artificial Sequence
  SYNTHESIZED
tccttgatggacaaggagtgt

SEQ ID NO: 6 - DNA, 19 bp, Artificial Sequence
  SYNTHESIZED
cccagtccatacggtacga

SEQ ID NO: 7 - DNA, 20 bp, Artificial Sequence
  SYNTHESIZED
taccccaatttccaatgctc

SEQ ID NO: 8 - DNA, 20 bp, Artificial Sequence
  SYNTHESIZED
tcttggtccttagccactcc

SEQ ID NO: 9 - DNA, 24 bp, Artificial Sequence
  SYNTHESIZED
agcactagtttcaacaccaagaaa

SEQ ID NO: 10 - DNA, 21 bp, Artificial Sequence
  SYNTHESIZED
cccagcctttcaaaattcttt

SEQ ID NO: 11 - DNA, 26 bp, Artificial Sequence
  SYNTHESIZED
ccatctatgagctacaagagaacaat

SEQ ID NO: 12 - DNA, 20 bp, Artificial Sequence
  SYNTHESIZED
tctgatccagtagcgacagc

SEQ ID NO: 13 - DNA, 20 bp, Artificial Sequence
  SYNTHESIZED
tatcgggtgacacagttgga

SEQ ID NO: 14 - DNA, 20 bp, Artificial Sequence
  SYNTHESIZED
tgaatggacttccccttcag

SEQ ID NO: 15 - DNA, 19 bp, Artificial Sequence
  SYNTHESIZED
gcatcctcatggagcacat

SEQ ID NO: 16 - DNA, 18 bp, Artificial Sequence
  SYNTHESIZED
ctggtcagccaggagctt

SEQ ID NO: 17 - DNA, 18 bp, Artificial Sequence
  SYNTHESIZED
ccctgcaccaagagcaac

SEQ ID NO: 18 - DNA, 19 bp, Artificial Sequence
  SYNTHESIZED
ggacccttcagggtacagg

SEQ ID NO: 19 - DNA, 20 bp, Artificial Sequence
  SYNTHESIZED
caccctgagcaagaaggaat

SEQ ID NO: 20 - DNA, 23 bp, Artificial Sequence
  SYNTHESIZED
tgtcatttatgagggcttcattt

SEQ ID NO: 21 - DNA, 20 bp, Artificial Sequence
  SYNTHESIZED
aggctgtcgctacatcactg

SEQ ID NO: 22 - DNA, 19 bp, Artificial Sequence
  SYNTHESIZED
acccgtagggcgattacag

SEQ ID NO: 23 - DNA, 20 bp, Artificial Sequence
  SYNTHESIZED
gtgattctgatggcccttct

SEQ ID NO: 24 - DNA, 20 bp, Artificial Sequence
  SYNTHESIZED
atcttgccagggaatcttga

SEQ ID NO: 25 - DNA, 20 bp, Artificial Sequence
  SYNTHESIZED
tgtcccccaacttgagatgt

SEQ ID NO: 26 - DNA, 23 bp, Artificial Sequence
  SYNTHESIZED
tgtgcacttttattcaactggtc

SEQ ID NO: 27 - DNA, 20 bp, Artificial Sequence
  SYNTHESIZED
tgatgcctcttccaggtgtt

SEQ ID NO: 28 - DNA, 20 bp, Artificial Sequence
  SYNTHESIZED
gcctcctcatggctttttgc

SEQ ID NO: 29 - DNA, 23 bp, Artificial Sequence
  SYNTHESIZED
actcacctcttcagaacgaattg

SEQ ID NO: 30 - DNA, 23 bp, Artificial Sequence
  SYNTHESIZED
ccatctttggaaggttcaggttg

SEQ ID NO: 31 - DNA, 20 bp, Artificial Sequence
  SYNTHESIZED
ccacctcagacctgatccca

SEQ ID NO: 32 - DNA, 21 bp, Artificial Sequence
  SYNTHESIZED
cccctggaattggttgtcctg

SEQ ID NO: 33 - DNA, 25 bp, Artificial Sequence
  SYNTHESIZED
ccttctcctttgggctggtcatcca

SEQ ID NO: 34 - DNA, 25 bp, Artificial Sequence
  SYNTHESIZED
cagacactggcaacattgcggacac

SEQ ID NO: 35 - DNA, 23 bp, Artificial Sequence
  SYNTHESIZED
acgtgatgacaagattgacaagc

SEQ ID NO: 36 - DNA, 21 bp, Artificial Sequence
  SYNTHESIZED
gcgaggtaatttgtgcccttt

SEQ ID NO: 37 - DNA, 23 bp, Artificial Sequence
  SYNTHESIZED
ggtcatagaacacatcatggagg

SEQ ID NO: 38 - DNA, 19 bp, Artificial Sequence
  SYNTHESIZED
ggcctggcttatggtggtg

SEQ ID NO: 39 - DNA, 22 bp, Artificial Sequence
  SYNTHESIZED
cctctctctaatcagccctctg

SEQ ID NO: 40 - DNA, 21 bp, Artificial Sequence
  SYNTHESIZED
gaggacctgggagtagatgag

(19) United States
(12) Patent Application Publication
(10) Pub. No.: US 2020/0375929 A1
(43) Pub. Date: Dec. 3, 2020

(54) METHODS AND COMPOSITIONS FOR THE TREATMENT OF DISEASES ASSOCIATED WITH CANCER, INFLAMMATION, OR IMMUNE RESPONSE

(71) Applicant: Washington University, St. Louis, MO (US)

(72) Inventors: Maxim Artyomov, St. Louis, MO (US); Monika Bambouskova, St. Louis, MO (US); Vicky Lampropoulou, St. Louis, MO (US); Roland Dolle, St. Louis, MO (US)

(21) Appl. No.: 16/639,352

(22) PCT Filed: Aug. 14, 2018
(86) PCT No.: PCT/US2018/046767

§ 371 Date: Feb. 14, 2020

Related U.S. Application Data
(60) Provisional application No. 62/545,154, filed on Aug. 14, 2017.
(60) Provisional application No. 62/545,118, filed on Aug. 14, 2017.
(60) Provisional application No. 62/654,698, filed on Apr. 9, 2018.

(51) Int. Cl. A61K 31/194; A61K 31/22; A61P 35/00

(52) U.S. Cl. CPC A61K 31/194; A61P 35/00; A61K 31/22

(57) ABSTRACT

Among the various aspects of the present disclosure is the provision of composition comprising itaconate, malonate, n or a derivative thereof and uses thereof. The present disclosure provides for methods of treating Ikb-ζ associated disease, disorder, or conditions comprising administering a therapeutically effective amount of an itaconate, malonate, or a derivative thereof to a subject. In some embodiments, the therapeutically effective amount reduces or prevents tumor growth, inflammation, or an immune response. Another aspect of the present disclosure includes a method to suppress Ikb-ζ induction comprising administering an itaconate, malonate, or a derivative thereof.

### CROSS-REFERENCE TO RELATED APPLICATIONS

This application claims the benefit of U.S. Provisional Application 62/545,154, filed Aug. 14, 2017, U.S. Provisional Application No. 62/545,118, filed Aug. 14, 2017 and U.S. Provisional Application No. 62/654,698, filed Apr. 9, 2018, the disclosures of which are hereby incorporated by reference in their entirety.

### FIELD OF THE TECHNOLOGY

The present disclosure generally relates to methods and compositions for the treatment of diseases associated with cancer, inflammation, or immune response. In particular, the provided compositions and methods can comprise administering an itaconate, malonate, or derivative thereof to a subject in need. Further, the disclosure provides administration of dimethyl itaconate to down-regulate Ikb-ζ induction, therefore useful in treatment for Ikb-ζ associated diseases, such as psoriasis, multiple sclerosis, and lymphoma (e.g., ABC subtype of DLBL lymphoma) or to reduce the extent of tissue injury in cardiovascular infarction.

### BACKGROUND

Metabolic regulation emerged as a novel powerful principle guiding immune responses. Remodeling of the tricarboxylic acid (TCA) cycle is a metabolic adaptation mechanism accompanying inflammatory macrophage activation. During this process, endogenous metabolites can adopt regulatory roles that govern specific aspects of inflammatory response. One of the most significant metabolic signals comes from succinate, which regulates the downstream pro-inflammatory IL-1β-HIF-1a axis. At present, the regulatory mechanisms modulating succinate levels remain unknown.

### SUMMARY

One aspect of the present disclosure is directed to the provision of a composition comprising an itaconate, malonate, or derivative thereof and uses thereof. The present disclosure provides for methods of treating Ikb-ζ associated disease, disorder, or conditions comprising administering a therapeutically effective amount of an itaconate, malonate, or derivative thereof to a subject. In some embodiments, the therapeutically effective amount reduces or prevents tumor growth, inflammation, or an immune response. Another aspect of the present disclosure includes a method to suppress Ikb-ζ induction comprising administering an Ikb-ζ modulation agent. Another aspect of the present disclosure includes a method of inhibiting tumor growth comprising administering an Ikb-ζ modulation agent. In some embodiments, the Ikb-ζ modulation agent comprises itaconate, itaconic acid, dimethyl itaconate (DI), 4-methyl itaconate, 3-(ethoxycarbonyl)but-3-enoic acid, 4-ethoxy-2-methylene-4-oxobutanoic acid, 4-octyl itaconate, dimethyl fumarate (DMF), diethyl malonate (DEM), dimethyl malonate, malonate, malonic acid, 2-methylenesuccinic acid, monoethylitaconate, 2-methyl fumaric acid, fuamaric acid, or a derivative or a pharmaceutically acceptable salt thereof, including all tautomers and stereoisomers.

In one aspect the itaconate, malonate, or a derivative thereof comprises a compound of formula I:

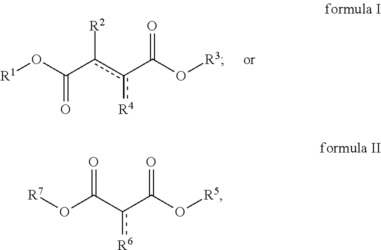

or a pharmaceutically acceptable salt thereof, including all tautomers and stereoisomers thereof wherein,

R¹ is hydrogen, unsubstituted or substituted alkyl; unsubstituted or substituted alkenes; or unsubstituted or substituted alkynes;

R² is hydrogen, unsubstituted or substituted alkyl; unsubstituted or substituted alkenes; or unsubstituted or substituted alkynes;

R³ is hydrogen, unsubstituted or substituted alkyl; unsubstituted or substituted alkenes; or unsubstituted or substituted alkynes;

R⁴ is hydrogen, unsubstituted or substituted alkyl; unsubstituted or substituted alkenes; or unsubstituted or substituted alkynes;

R⁵ is hydrogen, unsubstituted or substituted alkyl; unsubstituted or substituted alkenes; or unsubstituted or substituted alkynes;

R⁶ is hydrogen, unsubstituted or substituted alkyl; unsubstituted or substituted alkenes; or unsubstituted or substituted alkynes; and

R⁷ is hydrogen, unsubstituted or substituted alkyl; unsubstituted or substituted alkenes; or unsubstituted or substituted alkynes; wherein

R¹, R², R³, R⁴, R⁵, R⁶ or R⁷ can be optionally substituted with one or more groups independently selected from the group consisting of hydroxyl; C₁₋₁₀ alkyl hydroxyl; amine; C₁₋₁₀ carboxylic acid; C₁₋₁₀ carboxyl; straight chain or branched C₁₋₁₀ alkyl, optionally containing unsaturation; a C₂₋₆ cycloalkyl optionally containing unsaturation or one oxygen or nitrogen atom; straight chain or branched C₁₋₁₀ alkyl amine; heterocyclyl; heterocyclic amine; and aryl comprising a phenyl; heteroaryl containing from 1 to 4 N, O, or S atoms; unsubstituted phenyl ring; substituted phenyl ring; unsubstituted heterocyclyl; and substituted heterocyclyl, wherein

the unsubstituted phenyl ring or substituted phenyl ring can be optionally substituted with one or more groups independently selected from the group consisting of hydroxyl; C₁₋₁₀ alkyl hydroxyl; amine; C₁₋₁₀ carboxylic acid; C₁₋₁₀ carboxyl; straight chain or branched C₁₋₁₀ alkyl, optionally containing unsaturation; straight chain or branched C₁₋₁₀ alkyl amine, optionally containing unsaturation; a C₂₋₆ cycloalkyl optionally containing unsaturation or one oxygen or nitrogen atom; straight chain or branched C₁₋₁₀ alkyl amine; heterocyclyl; heterocyclic amine; aryl comprising a phenyl; and heteroaryl containing from 1 to 4 N, O, or S atoms; and the unsubstituted heterocyclyl or substituted heterocyclyl can be optionally substituted with one or more groups independently selected from the group consisting of hydroxyl; C₁₋₁₀ alkyl hydroxyl; amine; C₁₋₁₀ carboxylic acid; C₁₋₁₀ carboxyl; straight chain or branched C₁₋₁₀alkyl, optionally containing unsaturation; straight chain or branched C₁₋₁₀ alkyl amine, optionally containing unsaturation; a C₂₋₆ cycloalkyl optionally containing unsaturation or one oxygen or nitrogen atom; heterocyclyl; straight chain or branched C₁₋₁₀ alkyl amine; heterocyclic amine; and aryl comprising a phenyl; and heteroaryl containing from 1 to 4 N, O, or S atoms.

Other objects and features will be in part apparent and in part pointed out hereinafter.

### BRIEF DESCRIPTION OF THE FIGURES

The application file contains at least one drawing executed in color. Copies of this patent application publication with color drawing(s) will be provided by the Office upon request and payment of the necessary fee.

FIG. 1A, FIG. 1B, FIG. 1C, FIG. 1D, FIG. 1E, FIG. 1F and FIG. 1G show a series of images and graphs. (FIG. 1A) Expression of selected genes from RNA-Seq dataset generated from BMDMs treated or not with 250 μM DI for 12 and stimulated with LPS 100 ng/ml for indicated times. (FIG. 1B) Structure of DI. (FIG. 1C) Nrf2 protein detection in whole cell lysates prepared from BMDM treated as in A and stimulated with LPS 100 ng/ml for indicated times. (FIG. 1D) BMDMs were treated with 250 μM DI for indicated times and production of intracellular ROS was determined by H2DCFDA detection by flow cytometry. MFI for H2DCFDA signal in samples is shown. (FIG. 1E) Total content of GSH in extracts prepared from BMDMs treated with 250 μM DI for indicated times. (FIG. 1F) GSH/GSSG ratio in extracts from BMDMs treated with 250 μM DI for indicated times. (FIG. 1G) BMDM were treated as in A and stimulated with 100 ng/ml LPS for 24 h. In some samples NAC was added at the time of DI addition (NAC_12) and in some samples NAC was added at the time of stimulation with LPS (NAC_0). Production of IL6 in cell supernatants was detected by ELISA.

FIG. 2A, FIG. 2B, FIG. 2C, FIG. 2 D, FIG. 2E, FIG. 2F, FIG. 2G. FIG. 2H, FIG. 2I, FIG. 2J and FIG. 2K show a series of images and graphs comparing BMDM cells under treatment conditions. (FIG. 2A) BMDM were treated with DI 250 μM for 12 h and stimulated with LPS for 30 min. Cells were fixed, permeabilized and stained for nuclei (DARI) p65 and F-actin (in green). (FIG. 2B) BMDM were treated with various DI concentration (100, 150, 250 μM) for 12 h and stimulated with LPS for indicated times. Phosphorylated IKK and total Ikbζ (FIG. 2C) were detected by western blot in whole cell lysates. (FIG. 2D) BMDM were treated as in FIG. 2A and stimulated with 100 ng/ml LPS for indicated times. Ikbζ protein levels were detected by western blot. GAPDH was used as loading control (FIG. 2E) BMDM were treated as in FIG. 2A and stimulated with 100 ng/ml LPS for indicated times. Ikbζ expression mRNA was analyzed by qPCR. (FIG. 2F) BMDM were treated as in FIG. 2A and stimulated with 100 ng/ml LPS for indicated times. In some samples NAC was added at the time of DI addition (NAC_12) and in some samples NAC was added at the time of stimulation with LPS (NAC_0). Ikbζ protein levels were detected by western blot. (FIG. 2G) BMDM were treated as in FIG. 2A and stimulated with LPS for indicated times. In some samples 10 μM MG132 was added at the time of stimulation. Ikbζ protein levels were detected by western blot. (FIG. 2H) Human CD14+ monocytes were treated with 250 μM DI for 12 h and stimulated with 100 ng/ml LPS for indicated times. Ikbζ protein levels were detected by western blot. (FIG. 2I) Human CD14+ monocytes treated as in FIG. 2G were stimulated with 100 ng/ml LPS for 24 h. Cytokine levels in cell supernatants were detected by ELISA. (FIG. 2J) GFP or GFP fused to 3-UTR sequence of Nfkbiz mRNA were introduced to BV2 cells by lentiviral transduction. Positive clones were selected by puromycin. Cells were then treated with 250 μM DI for 12 h and stimulated with 100 ng/ml LPS for 1 h. GFP fluorescence was determined by flow cytometry. (FIG. 2K) BMDM were treated as in FIG. 2A and stimulated with 100 ng/ml LPS for indicated times. Regnase-1 protein levels were detected by western blot.

FIG. 3A and FIG. 3B show a series of Western blots. (FIG. 3A) BMDM derived from WT or Nrf2 KO mice were treated with 250 μM DI for 12 h and stimulated with 100 ng/ml LPS for indicated times. Ikb-ζ protein levels were detected by western blot. GAPDH was used as loading control. (FIG. 3B) BMDMs were treated as in FIG. 3A and STAT3 phosphorylation at Y705 or S727 sites was determined by western blot. GAPDH was used as loading control.

FIG. 4A. FIG. 4B, FIG. 4C, FIG. 4D and FIG. 4E show a series of graphs. (FIG. 4A) HaCat cells were treated with DI as indicated for 12 h and then stimulated with 100 ng/ml IL17A for indicated times. Ikb-ζ, STAT3 and STAT3 phosphorylation at Y705 were detected by western blot. GAPDH was used as loading control. (FIG. 4B) HaCat cells were treated as in A and cell viability was determined by propidium iodide staining and flow cytometry. (FIG. 4C) Human primary keratinocytes were treated with DI as indicated for 12 h and then stimulated with 100 ng/ml IL17 for indicated times. Ikb-ζ protein levels were detected by western blot. GAPDH was used as loading control. (FIG. 4D) Human primary keratinocytes were treated as in FIG. 4C and cell viability was determined by propidium iodide staining and flow cytometry. (FIG. 4E) Human primary keratinocytes were treated and stimulated as in FIG. 4C and mRNA expression of selected genes was analyzed by qPCR.

FIG. 5A and FIG. 5B is a series of images and a graph. (FIG. 5A) DI was administered to mice daily, which led to significant improvement of the psoriatic pathology. (FIG. 5B) The same daily regimen of DI or vehicle (ve) administration was used in vivo as in the EAE model of the mice and found that DI prevented clinical development of EAE.

FIG. 6 is a bar graph showing DLBCL cell lines cultured at the presence of indicated concentrations of DI. Media containing DI was refreshed every day for four days in total. Cell viability was determined by resazurin assay at the day four.

FIG. 7A and FIG. 7B is a series of Western blots and bar graphs. (FIG. 7A) BMDM were treated with DMF at different concentrations for 12 h and stimulated with 100 ng/ml LPS for indicated times. Ikb-ζ and pro-IL1β protein levels and STAT3 activation were determined by western blot. GAPDH was used as loading control. (FIG. 7B) BMDM were treated as in FIG. 7A and stimulated with 100 ng/ml LPS for indicated times. Cytokine production in cell supernatants was determined by ELISA.

FIG. 8A, FIG. 8B, FIG. 8C, FIG. 8D, FIG. 8E, FIG. 8F, FIG. 8G and FIG. 8H show a series of graphs and images including the structure of DI and comparison transcriptional profiles. (FIG. 8A) Structure of DI. (FIG. 8B) Comparison of transcriptional profiles of cKeap KO BMDMs and BMDMs treated with 250 μM DI for 12 h. (FIG. 8C) Triggering of Nrf2 expression and expression of Nrf2-targets (Nqo1, HO-1) in BMDMs treated as in B. (FIG. 8D) Analysis of intracellular DI uptake and identification of DI-glutathione adduct. (FIG. 8E) ROS production measured by detection of CM-H2DCFDA in BV2 cells treated with DI as in B. (FIG. 8F) Glutathione depletion and GSH/GSSG ratio (FIG. 8G) in BMDMs treated with DI as in FIG. 8B (FIG. 8H) N-acetylcysteine (NAC) neutralizes effect of DI on cytokine production. BMDMs were treated with 250 μM DI and stimulated with LPS for 12 h. In some samples NAC was added simultaneously with DI.

FIG. 9A, FIG. 9B and FIG. 9C show a series of Western blot images and fluorescent microscope images. (FIG. 9A) BMDMs were treated with 250 μM DI for 12 h and stimulated with LPS. Loss of IRAK1 detection upon LPS refers to its K63 ubiquitination that correlates with activation. DI affected phosphorylation of IKK in time-dependent manner. (FIG. 9B) Degradation of Ikbζ is also not affected by DI treatment. (FIG. 9C) BMDMs were treated with DI as in A and fixed, permeabilized and stained for p65, F-Actin and nuclei. DI pretreatment does not inhibit nuclear translocation of p65 upon LPS stimulation.

FIG. 10A, FIG. 10B, FIG. 10C, FIG. 10D, FIG. 10E, FIG. 10F, FIG. 10G and FIG. 10H show a series of Western blots images and graphs. (FIG. 10A) Detection of Ikbζ expression in BMDMs treated with DI for 12 h and stimulated with LPS. (FIG. 10B) Dose dependent effect of DI on cytokine production in BMDMs pretreated with DI for 12 h and stimulated with LPS for 4 h. (FIG. 10C) Dose dependent inhibition of Ikbζ expression in in cell treated as in FIG. 10B and stimulated with LPS for 1 h. (FIG. 10D) Nfkbiz mRNA expression in Ikbζ protein expression in cells treated as in FIG. 10C. (FIG. 10E and FIG. 10G) Diminished Ikbζ protein expression is not due to the proteasomal degradation as shown by treatment of cells with MG132 or due to lysosomal/autophagy degradation as shown by treatment of cells with BafalomycinA (BafA). (FIG. 10F) Dimethyl malonate (DM), a succinyl dehydrogenase inhibitor, does not affect Ikbζ expression. BMDMs were treated with 10 mM DM for 12 h and stimulated with LPS. (FIG. 10H) Detection of Ikbζ expression in human CD14+ monocytes treated with DI for 12 h and stimulated with LPS.

FIG. 11A, FIG. 11B, FIG. 11C and FIG. 11D show a series of images and graphs. (FIG. 11A) Ikbζ expression in BMDMs treated with 250 μM DI for 12 h and stimulated with LPS. In some samples N-acetylcysteine was added simultaneously with DI (NAC_12) or at the time of LPS stimulation (NAC_0). (FIG. 11B) Ikbζ expression in Nrf2 deficient BMDMs. Cell were treated with 250 μM DI for 12 h prior to LPS stimulation. (FIG. 11C) Transcriptional comparison of DI treated WT and Nrf2 KO BMDM. Left panel shows signature pathways up-regulated and down-regulated by DI independent of Nrf2. (FIG. 11D) Most significantly up-regulated genes by DI in Nrf2-independent manner.

FIG. 12A, FIG. 12B, FIG. 12C, FIG. 12D, FIG. 12E and FIG. 12F show a series of Western blots and bar graphs. (FIG. 12A) shows Ikbζ expression in primary mouse keratinocytes that were treated with DI for 12 h and then stimulated with IL-17A (100 ng/ml). (FIG. 12B) Viability of mouse keratinocytes treated as in FIG. 12A. (FIG. 12C) qPCR analysis of gene expression in mouse keratinocytes treated as in FIG. 12A. (FIG. 12D) Ikbζ expression in primary human keratinocytes that were treated with DI for 12 h then stimulated with IL-17A (100 ng/ml). (FIG. 12E) Viability of mouse keratinocytes treated as in FIG. 12D. (FIG. 12F) qPCR analysis of gene expression in mouse keratinocytes treated as in FIG. 12D.

FIG. 13A and FIG. 13B is a series of images and bar graphs. (FIG. 13A) BIG mice were injected i.p. with a dose of DI and imiquimod (IMQ) was applied topically on ear skin daily for 7 days. Sections of imiquimod-treated ears from mice following 5 d of treatment are shown. (FIG. 13B) qPCR analysis of gene expression in skin tissue of mice treated as in FIG. 13A.

FIG. 14A, FIG. 14B, FIG. 14C, FIG. 14D, FIG. 14E, FIG. 14F, FIG. 14G, FIG. 14H and FIG. 14I depict a series of images and graphs showing that DI induces electrophilic stress in macrophages. (FIG. 14A) Nrf2 response genes in DI treated BMDMs. (FIG. 14B and FIG. 14C) Western blot of Nrf2 and Nrf2 targets in DI treated or LPS-stimulated BMDMs. (FIG. 14D) Structures of DI- or Ita-GSH. (FIG. 14E) DI-GSH levels in media of DI-treated BMDMs, mean, n=2 cultures. (FIG. 14F) Itaconate and Ita-GSH levels in BMDMs, mean±s.e.m, n=3 cultures. (FIG. 14G) GSH levels in DI-treated BMDMs, mean, n=2 experiments. (FIG. 14H and FIG. 14I) Cytokines in BMDMs treated as indicated and LPS stimulated for FIG. 14H, 4 h; FIG. 14I, 24 h; mean±s.e.m, n=3 experiments. Western blots are representatives of 3 experiments. Two-tailed t-test.

FIG. 15A, FIG. 15B, FIG. 15C, FIG. 15D, FIG. 15E, FIG. 15F, FIG. 15G, FIG. 15H, FIG. 15I and FIG. 15J depict a series of images and graphs showing DI-GSH and Ita-GSH detection and electrophilic stress response. (FIG. 15A) Transcriptional comparison of KpCKO and WT BMDMs and enrichment of DI gene signature. (FIG. 15B) DI reacting with thiol group in Michael reaction. (FIG. 15C) DI levels in media of BMDMs treated with DI for indicated time determined by GCMS, mean, n=2 cultures. (FIG. 15D) DI-GSH conjugate levels in media of BMDMs treated with DI for indicated time detected by LCMS, mean, n=2 cultures. Data from FIG. 14E are overlaid with data for cell free media. (FIG. 15E) DI-GSH conjugate levels in BMDMs treated with 13C5-labeled DI for indicated time or in their media detected by LCMS, mean, n=2 cultures. (FIG. 15F and FIG. 15G) Representative extracted ion chromatograms of FIG. 15F, DI-GSH detected in media of BMDMs treated with DI (6 h) compared to synthesized DI-GSH standard; FIG. 15G, Ita-GSH structure detected in BMDMs stimulated with LPS (24 h) compared to synthesized Ita-GSH standard; n=10 technical replicates. (FIG. 15H) ROS detection in BV2 cells treated with DI for indicated time determined by flow cytometry, mean, n=2 experiments. (FIG. 15I) Cytokine production in BMDMs treated with DI in presence of EtGSH and stimulated with LPS for 4 h, mean±s.e.m., n=3 experiments. (FIG. 15J) Western blot of HO-1 in BMDMs treated with DMF. Representative of 3 experiments. Two-tailed t test.

FIG. 16A, FIG. 16B, FIG. 16C, FIG. 16D, FIG. 16E, FIG. 16F, FIG. 16G, FIG. 16H and FIG. 16I show a series of images and graphs showing DI inhibits LPS-mediated IκBζ induction. (FIG. 16A) mRNA expression in LPS-stimulated BMDMs. Representative of 2 experiments. (FIG. 16B, FIG. 16C, FIG. 16G, FIG. 16H, FIG. 16I, and FIG. 16J) Western blot of IκBζ expression in BMDMs treated with DI, DMF, MI (FIG. 16H, 5 μM, FIG. 16J, 10 μM) or 3MI, LPS for 1 h or as indicated. (FIG. 16D) Relative levels of IκBζ protein and mRNA in BMDMs treated with DI, LPS 1 h, mean±s.e.m., n=3 experiments. (FIG. 16E) Scheme of DI action. (FIG. 16F) Structures of DMF and itaconate derivatives. (FIG. 16K) Densitometry of IκBζ from FIG. 16B and itaconate levels, mean, n=6 cultures. (FIG. 16I) Western blot of IκBζ expression in BMDMs tolerized in presence of BSO. Western blots are representative of 3 experiments; (FIG. 16H) 2 experiments.

FIG. 17A, FIG. 17B, FIG. 17C, FIG. 17D, FIG. 17E, FIG. 17F, FIG. 17G, FIG. 17H, FIG. 17I, FIG. 17J, FIG. 17K, FIG. 17L and FIG. 17M show a series of images and graphs showing that DI downregulates secondary transcriptional response to TLR stimulation. (FIG. 17A) Western blot of IκBζ expression in WT or Nfkbiz−/− BMDMs stimulated with LPS. (FIG. 17B) Cytokine production in WT and Nfkbiz−/− BMDM stimulated with LPS for 4 h, mean±s.e.m., n=3 experiments. (FIG. 17C) RNA-seq analysis of BMDMs treated with DI and stimulated with LPS and IFN-γ. (FIG. 17D) mRNA expression in WT and Nfkbiz−/− BMDMs treated with DI and stimulated with LPS for 4 h, mean±s.e.m., n=3 experiments. (FIG. 17E) Western blot of IκBζ expression in DI-treated BMDMs stimulated with LPS 1 h. (FIG. 17F) mRNA expression in human blood monocytes treated with DI and stimulated with LPS. (FIG. 17G) Western blot of IκBζ expression in human blood monocytes treated DI and stimulated with LPS. (FIG. 17H and FIG. 17I) Western blot of FIG. 17H, IκBα or FIG. 17I, IRAK1 expression and IKK phosphorylation in BMDMs treated with DI and stimulated with LPS. (FIG. 17J) p65 localization in DI-treated, LPS-stimulated BMDMs. DAPI, nuclei. Bars 25 μM.

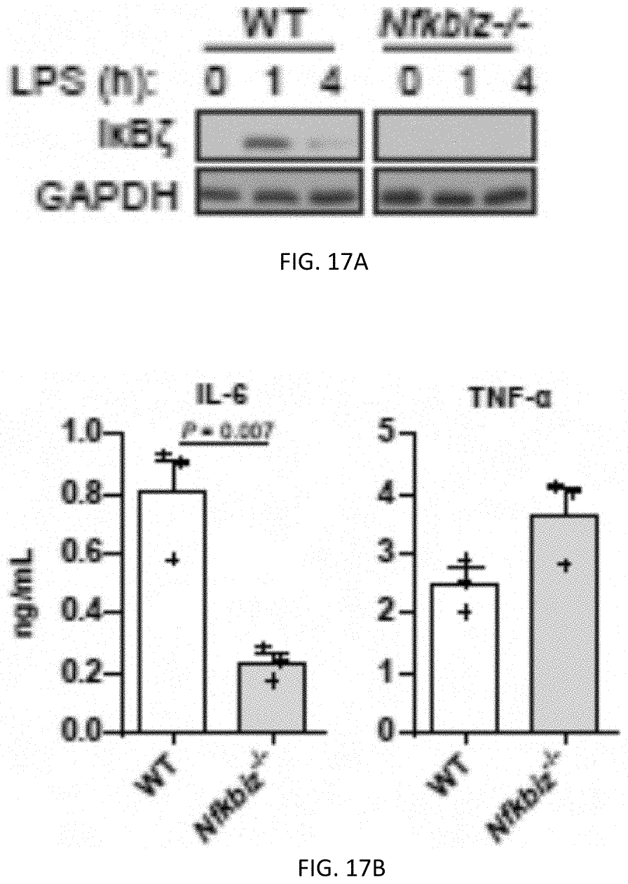

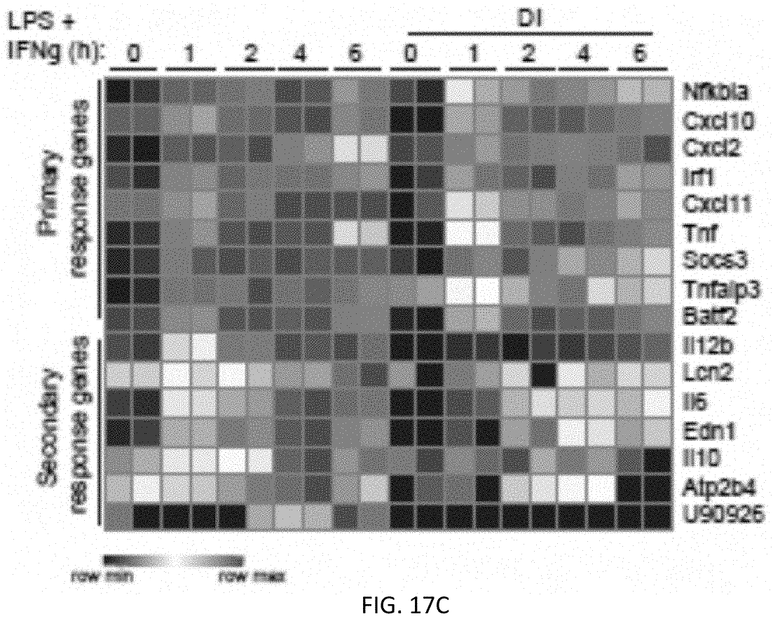

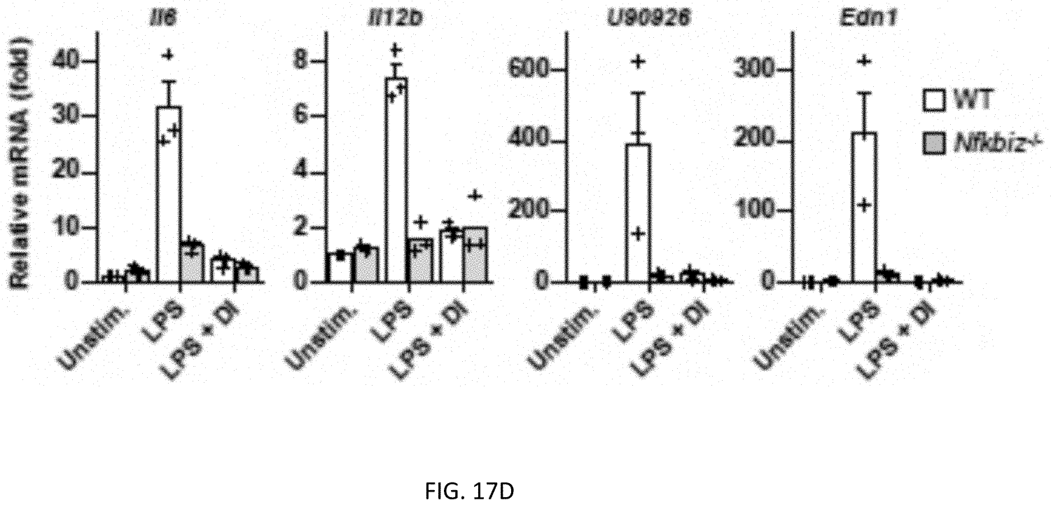

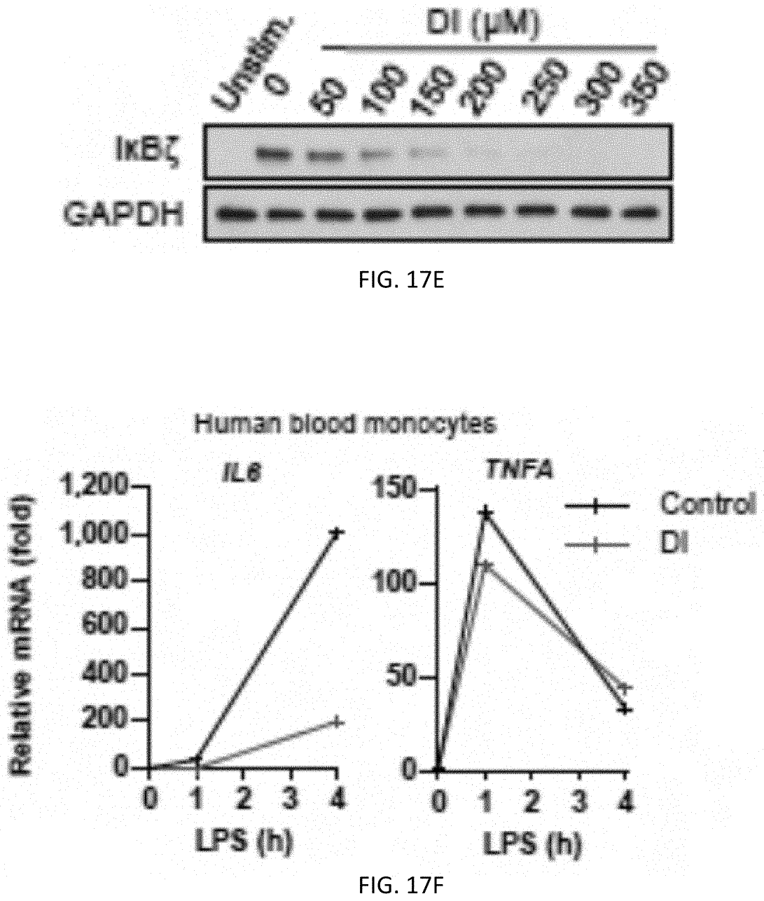

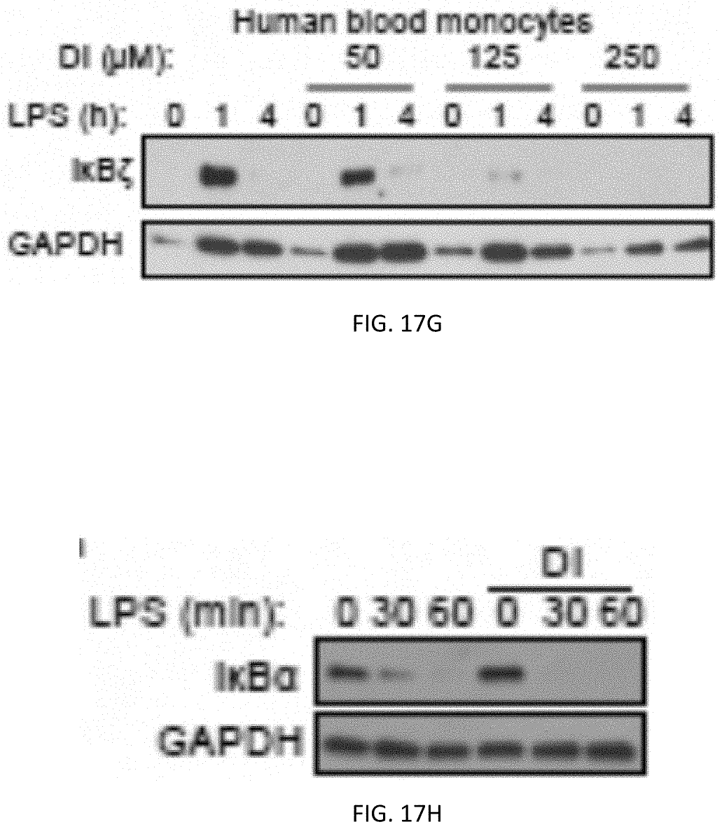

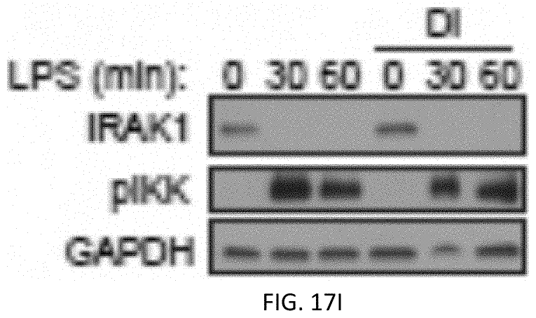

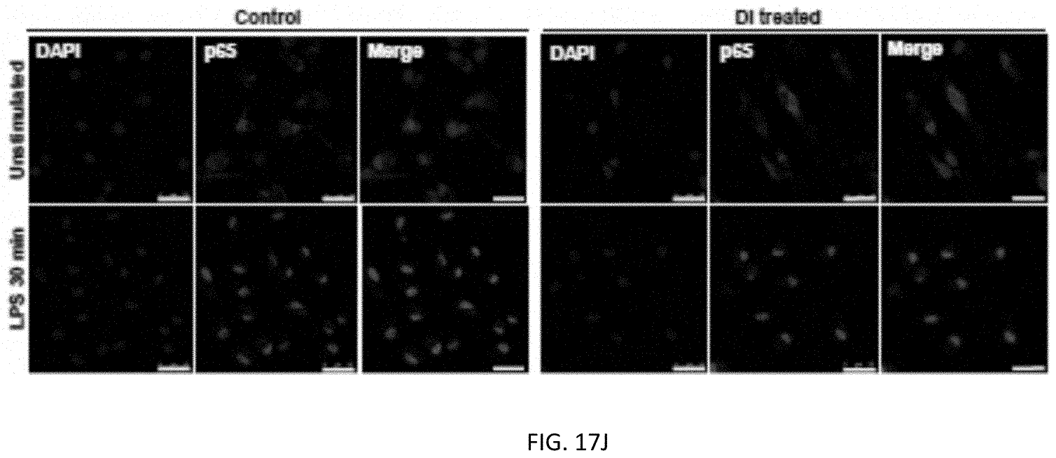

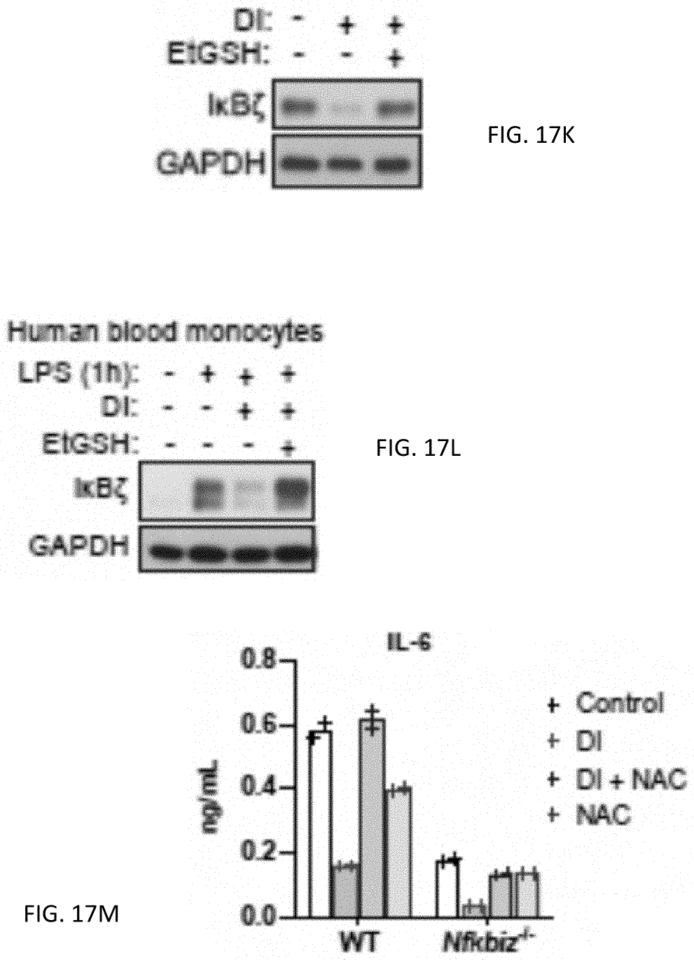

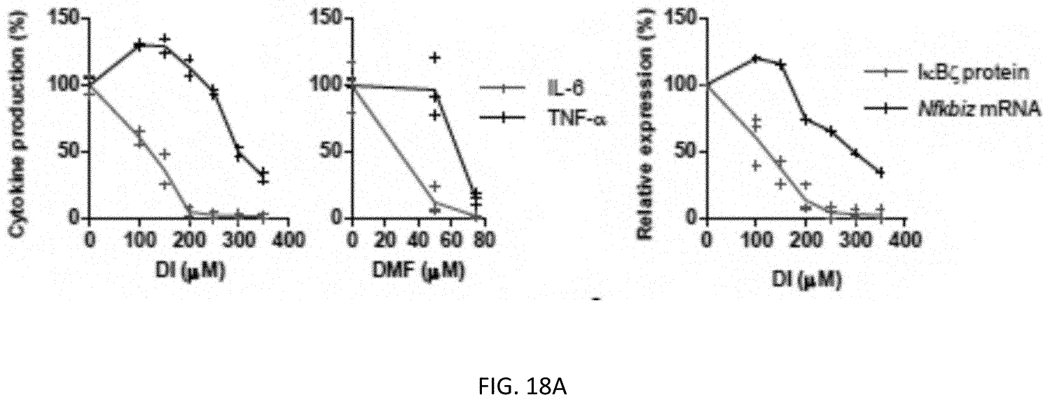

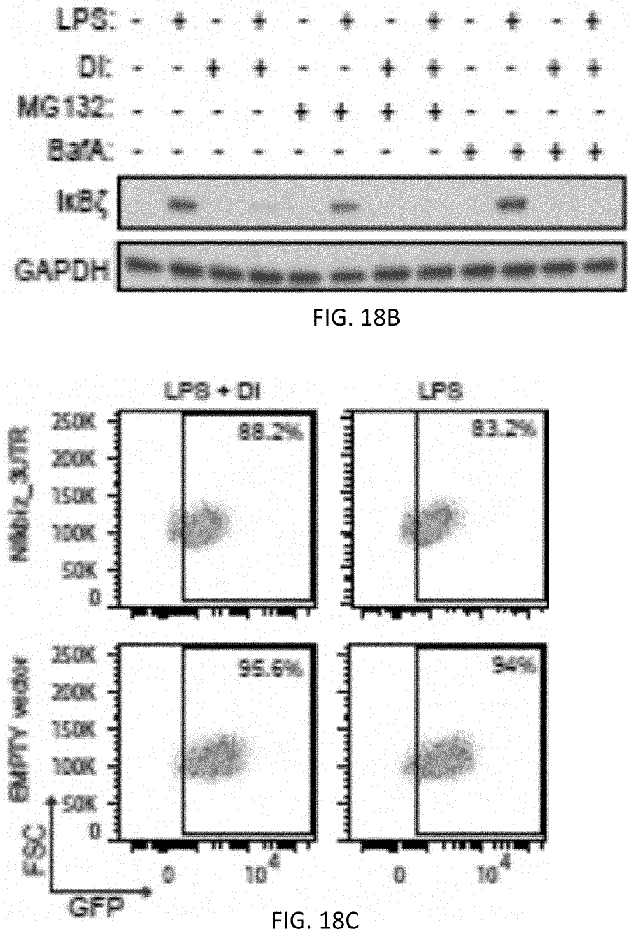

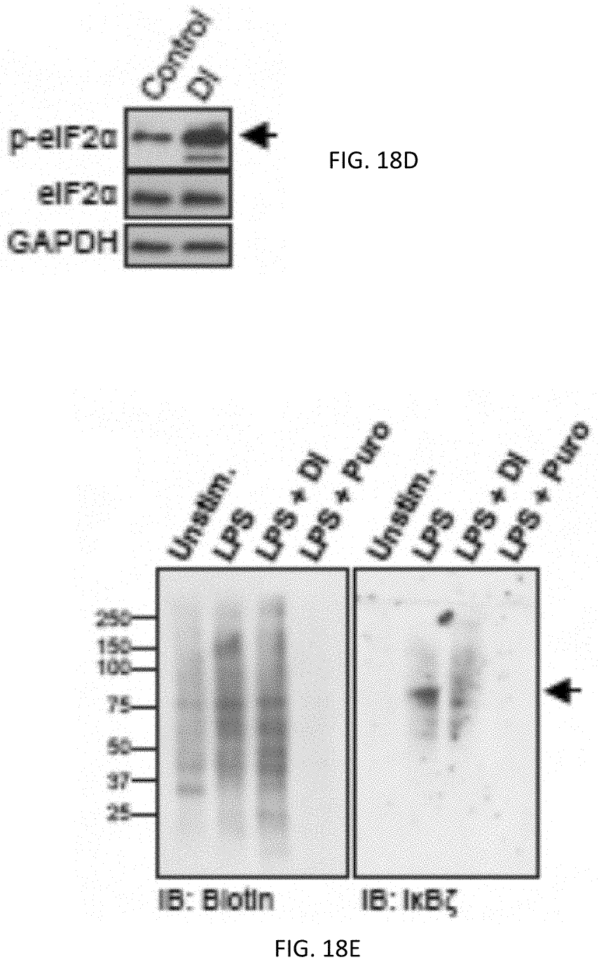

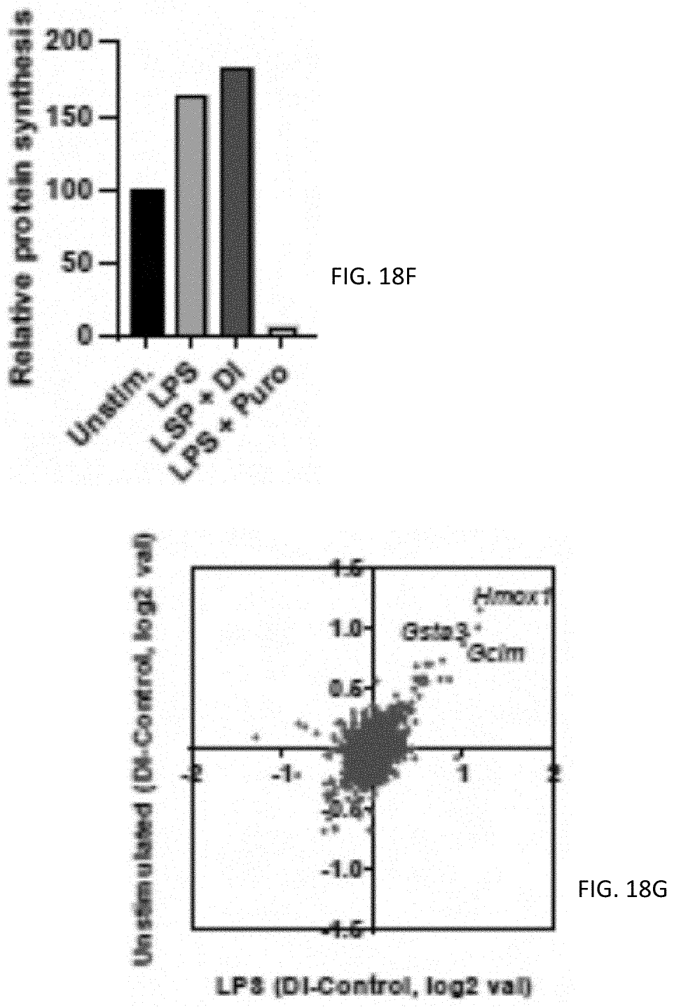

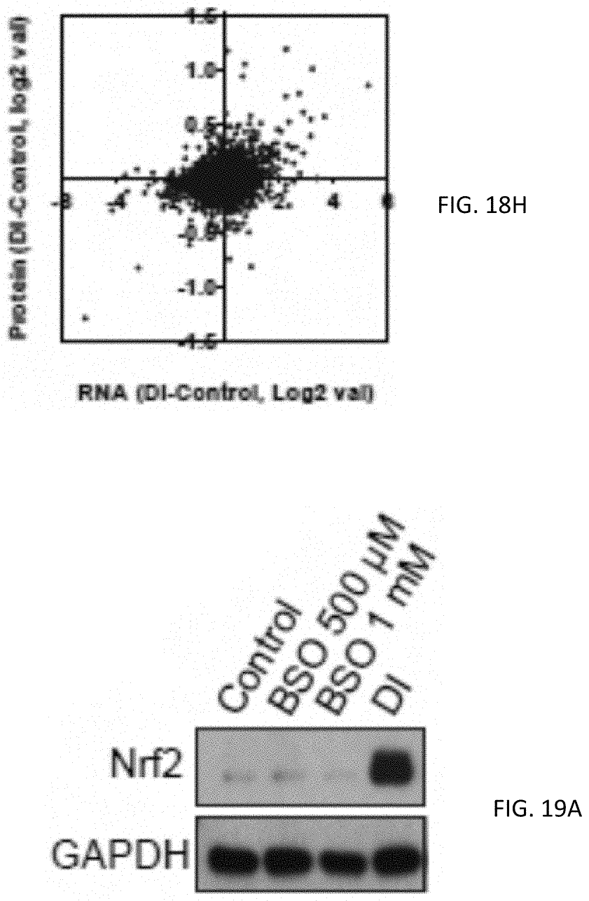

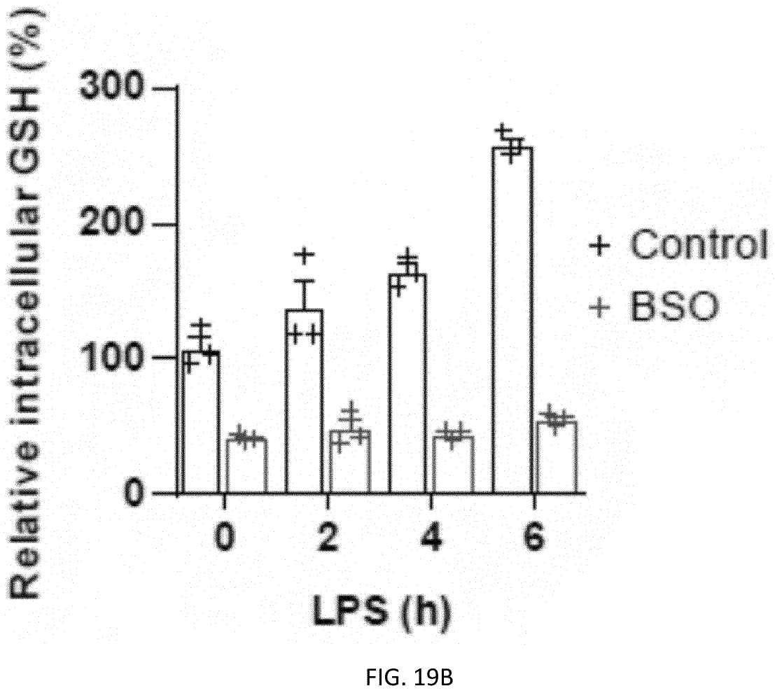

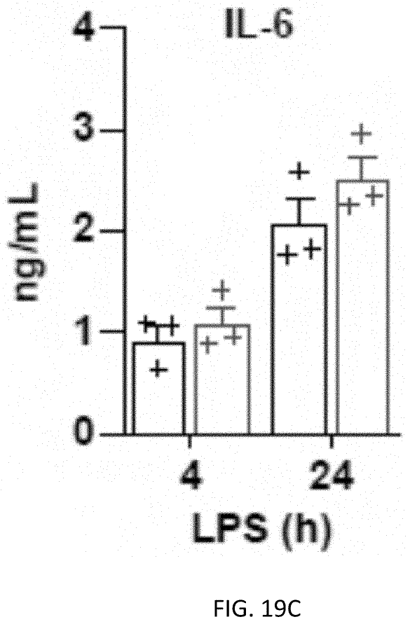

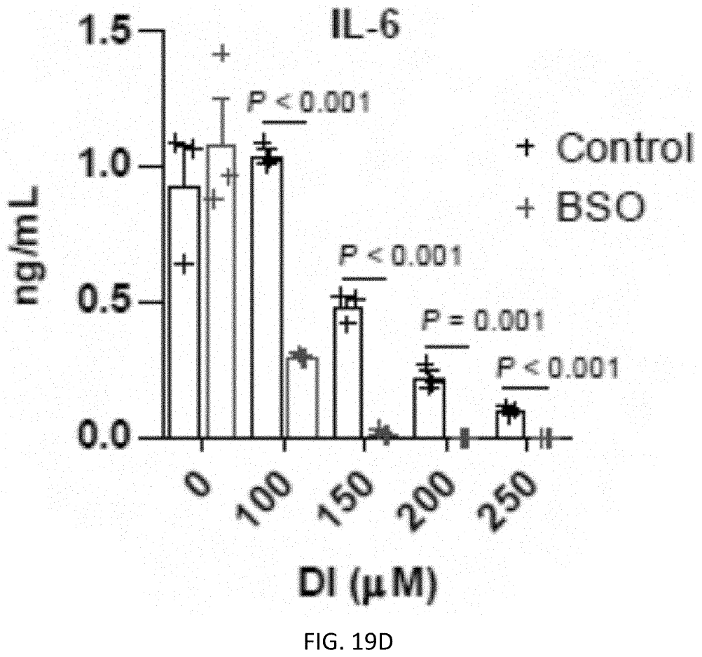

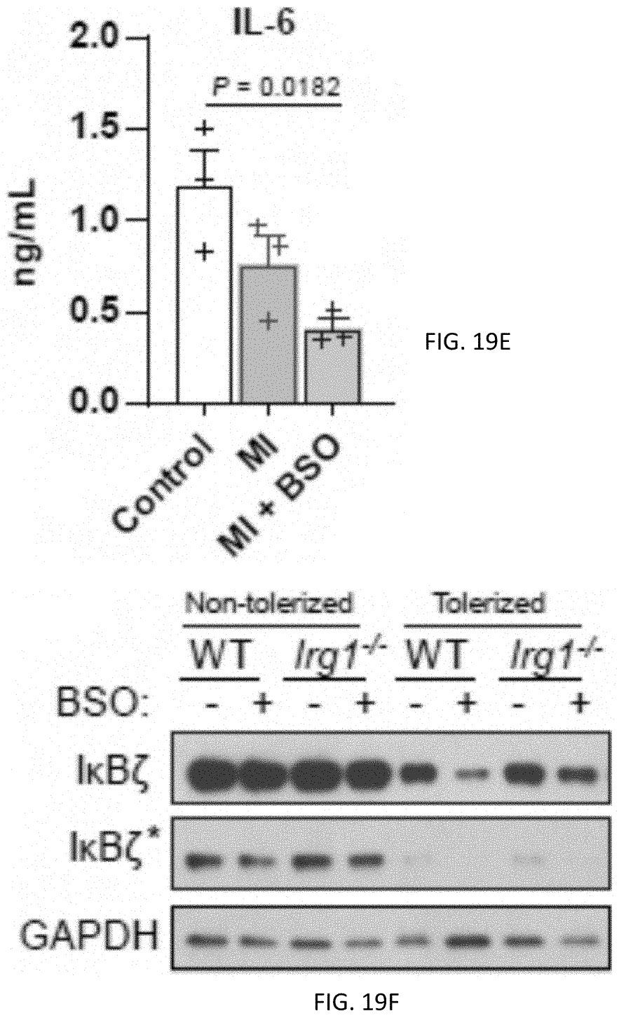

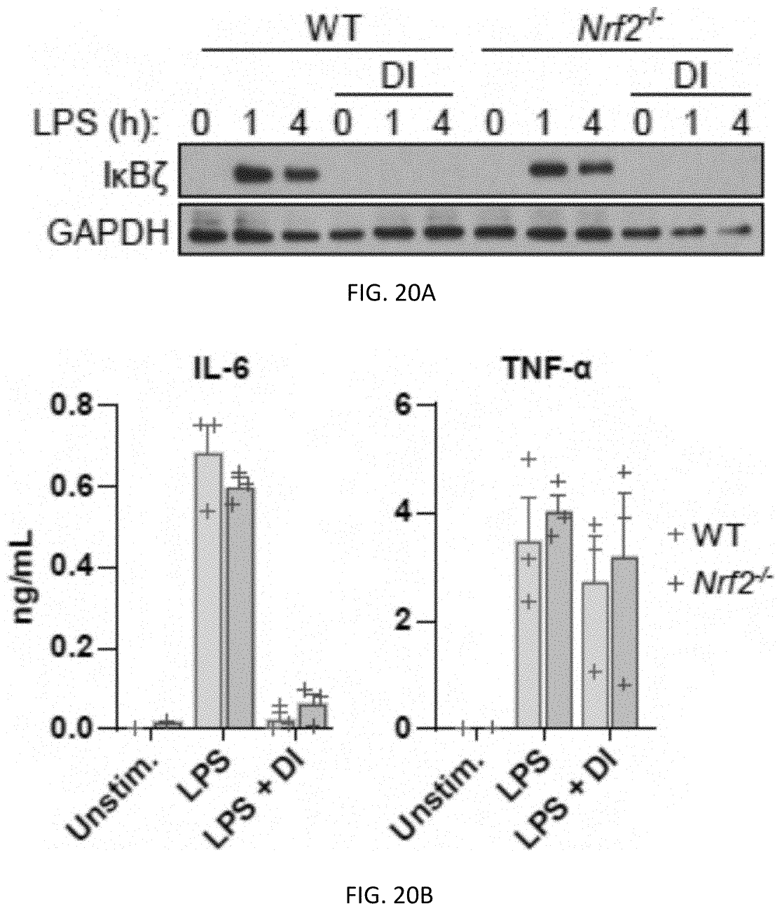

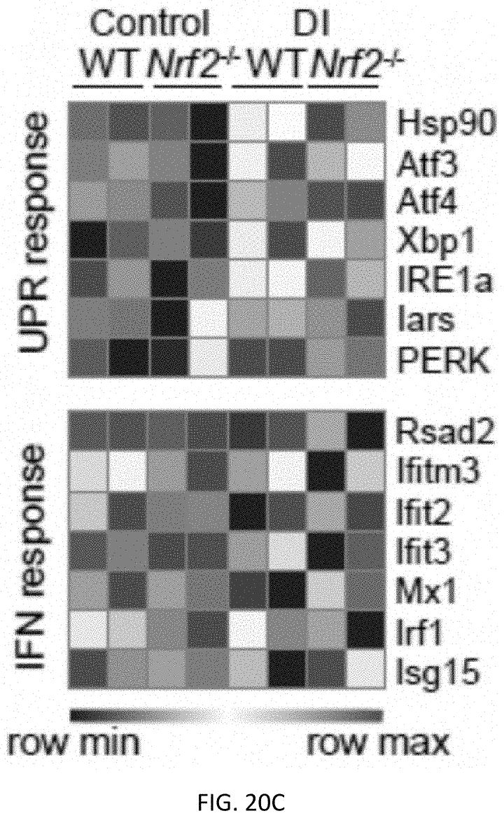

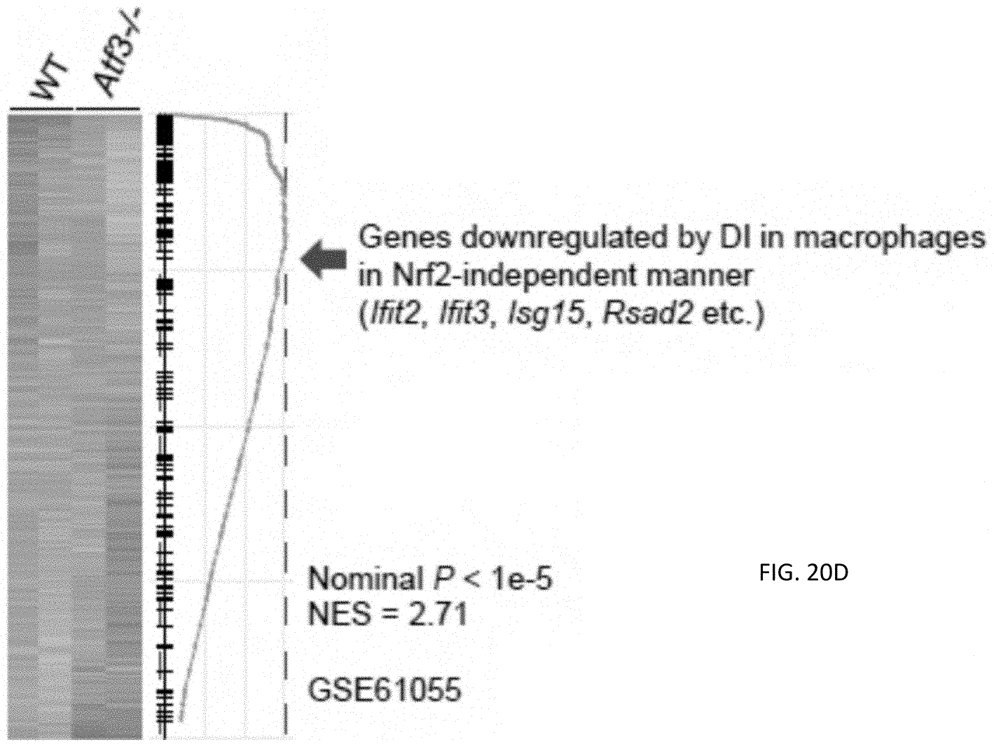

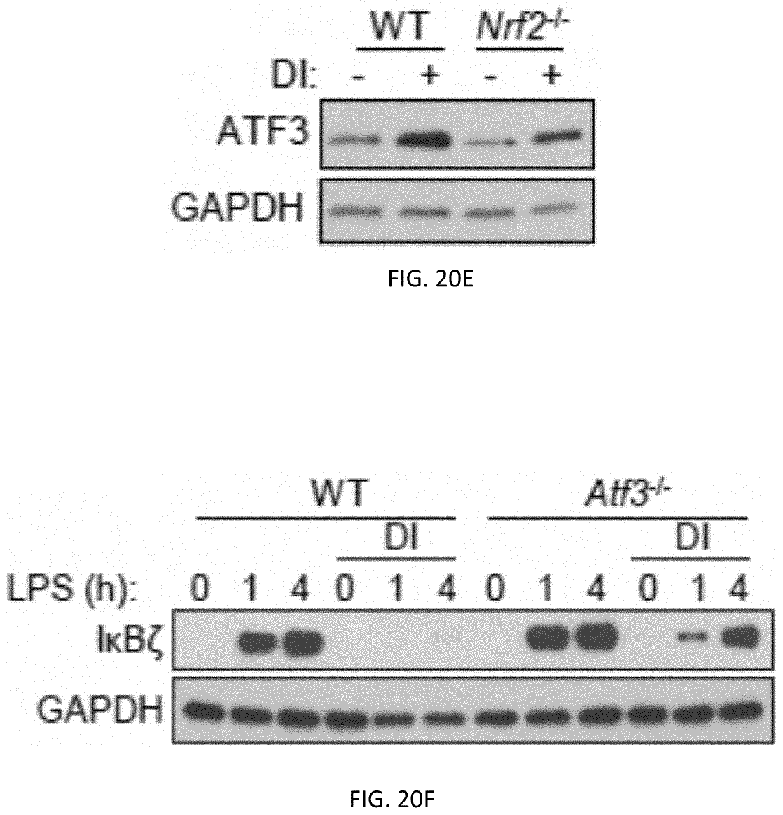

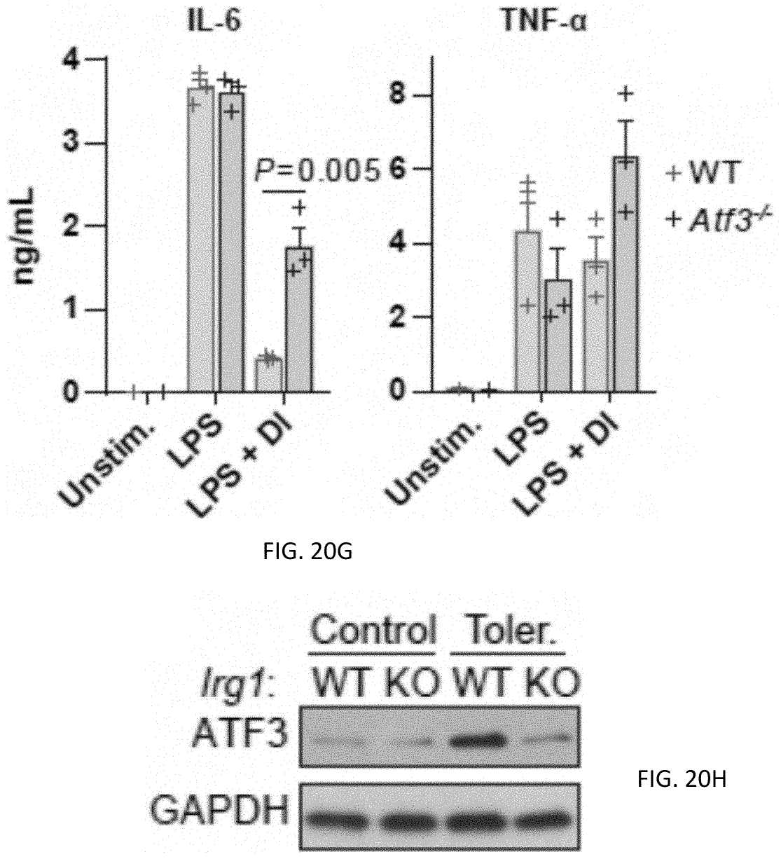

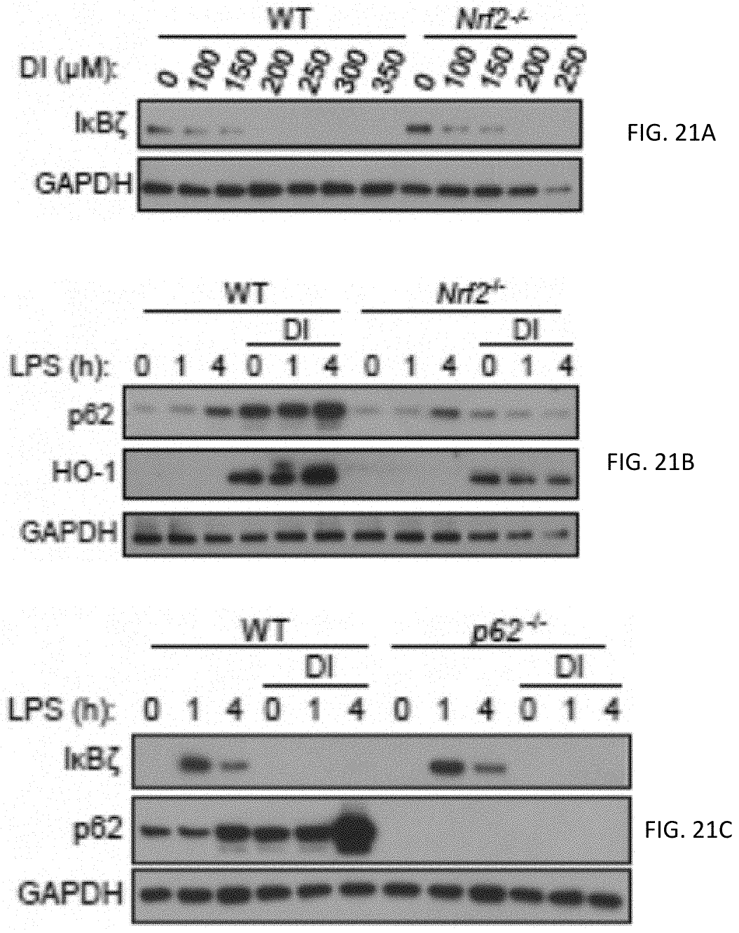

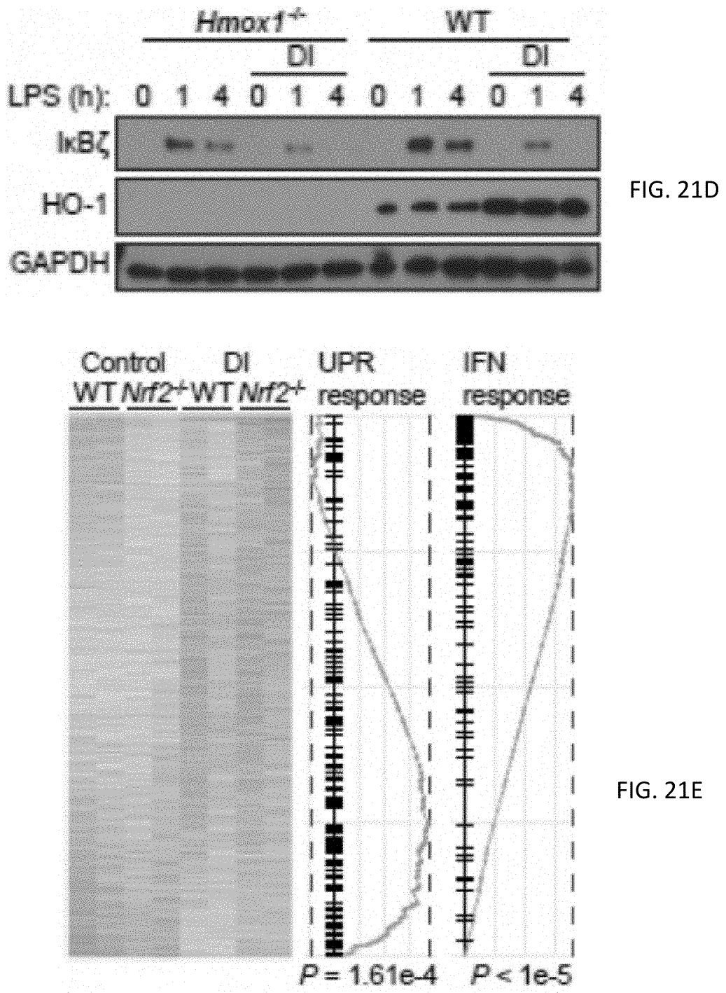

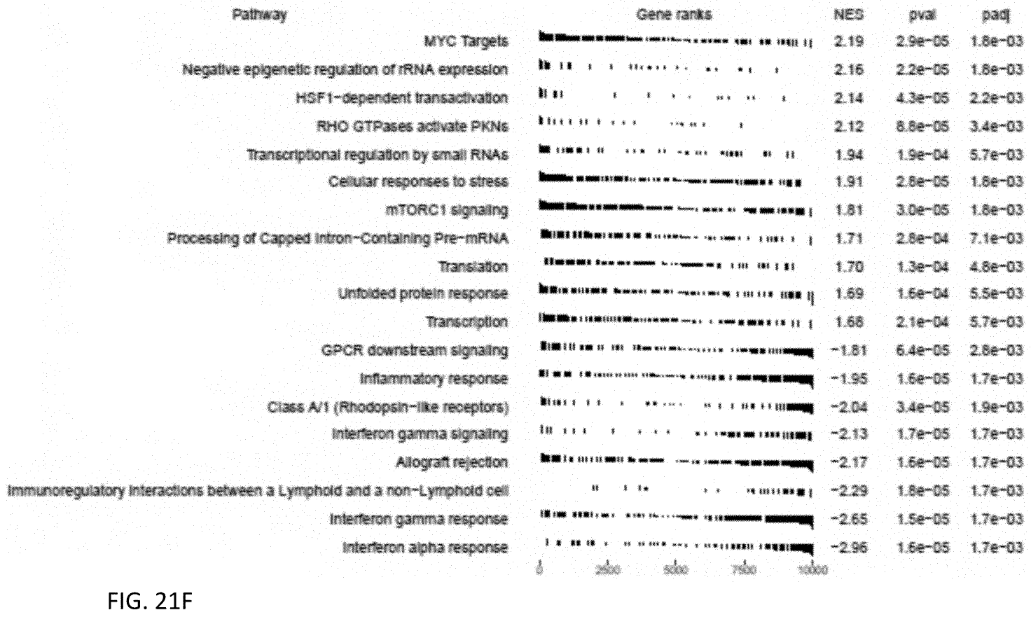

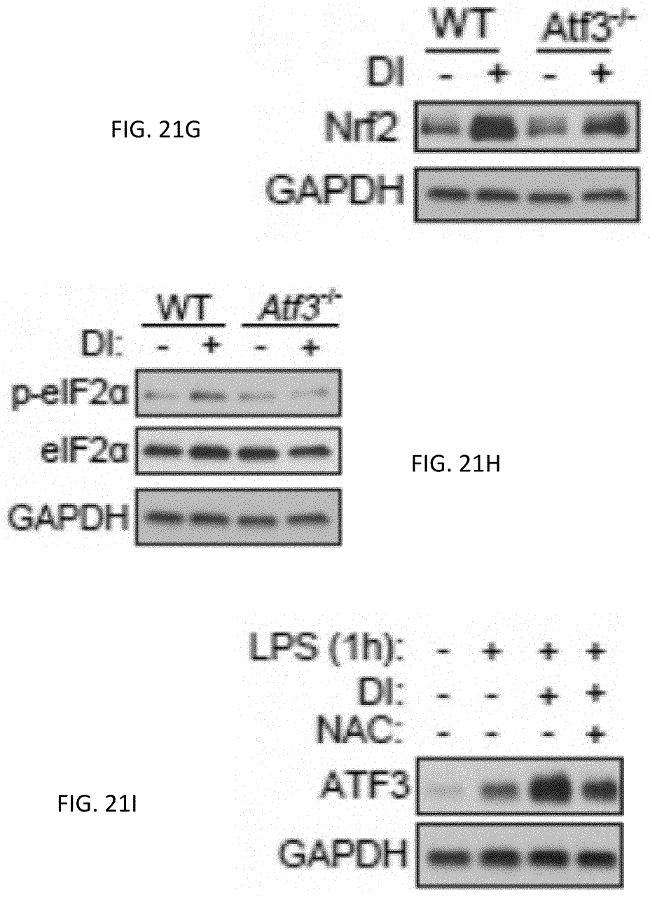

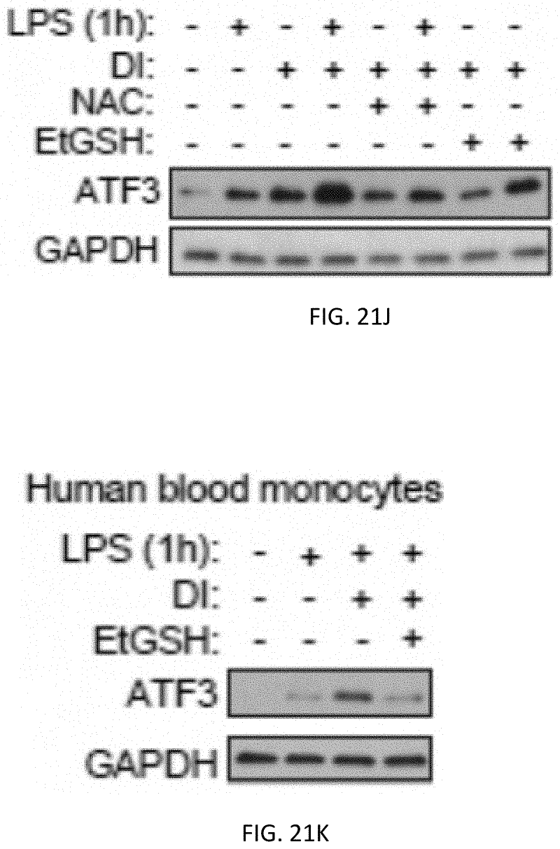

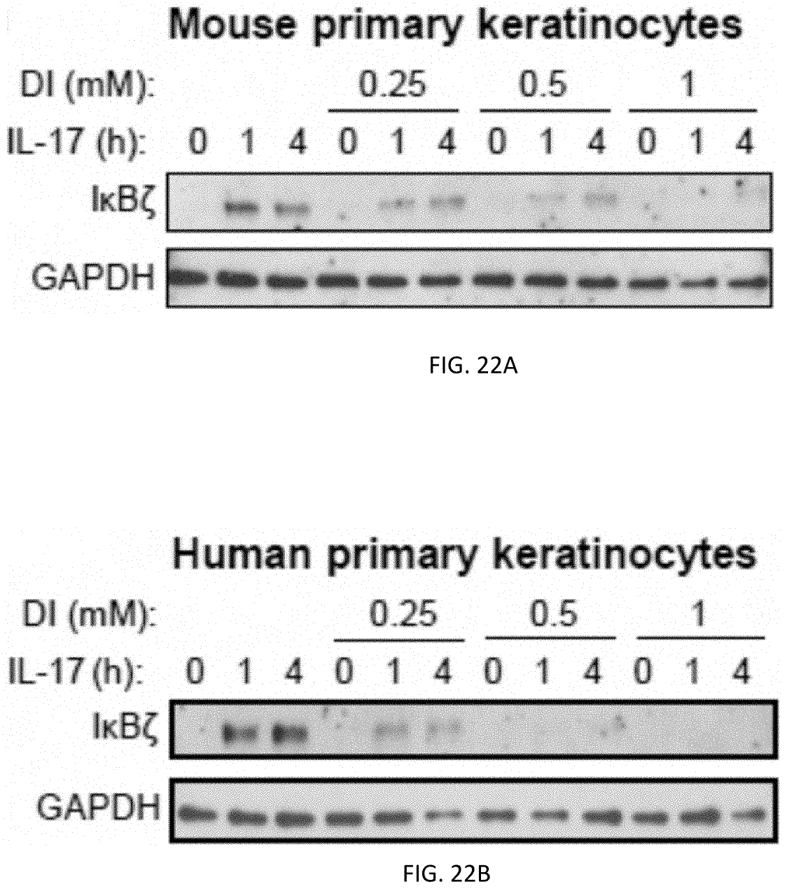

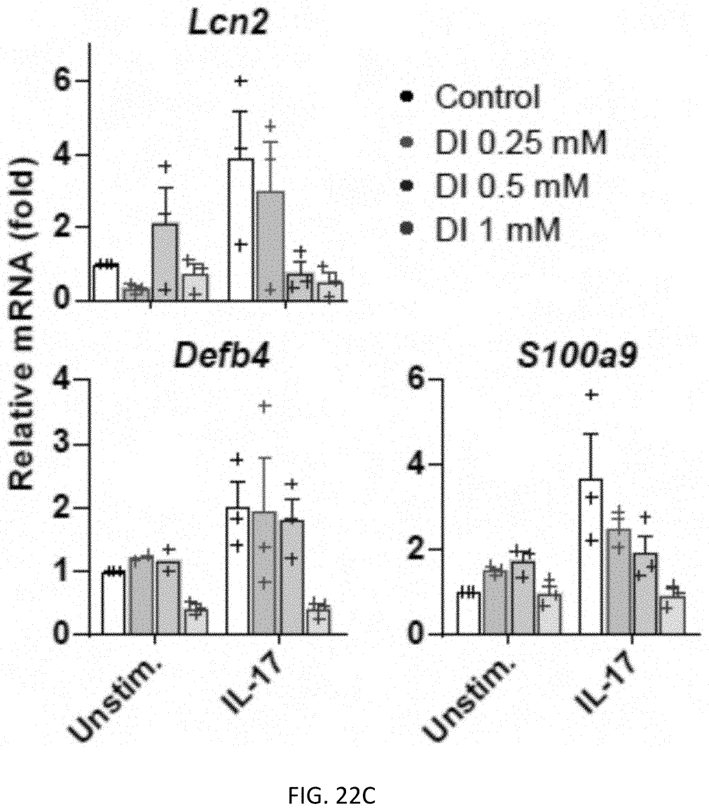

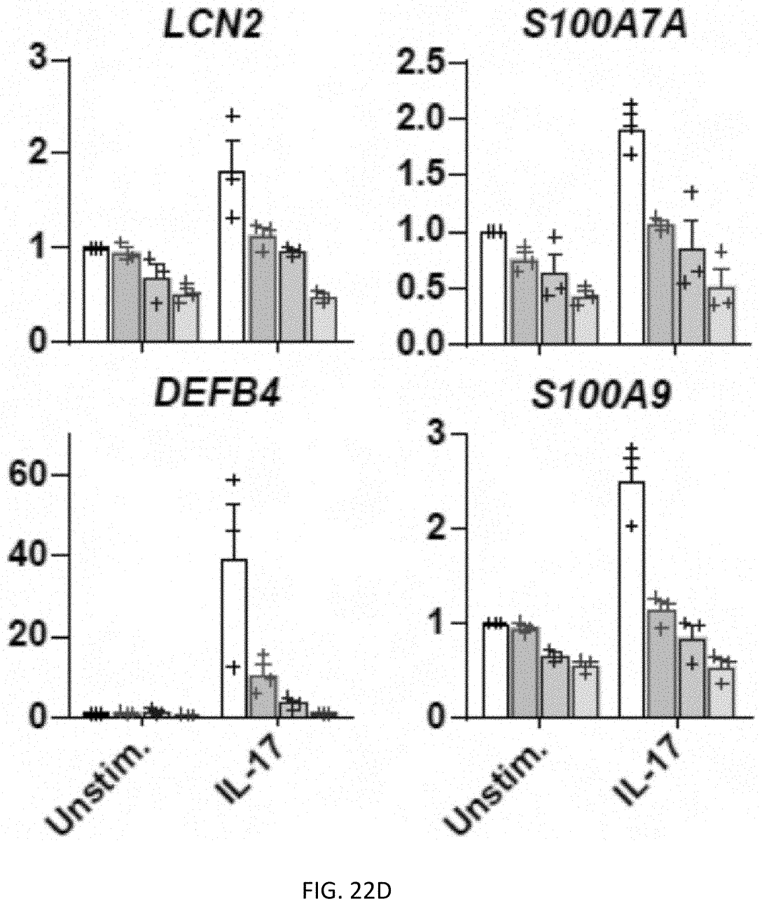

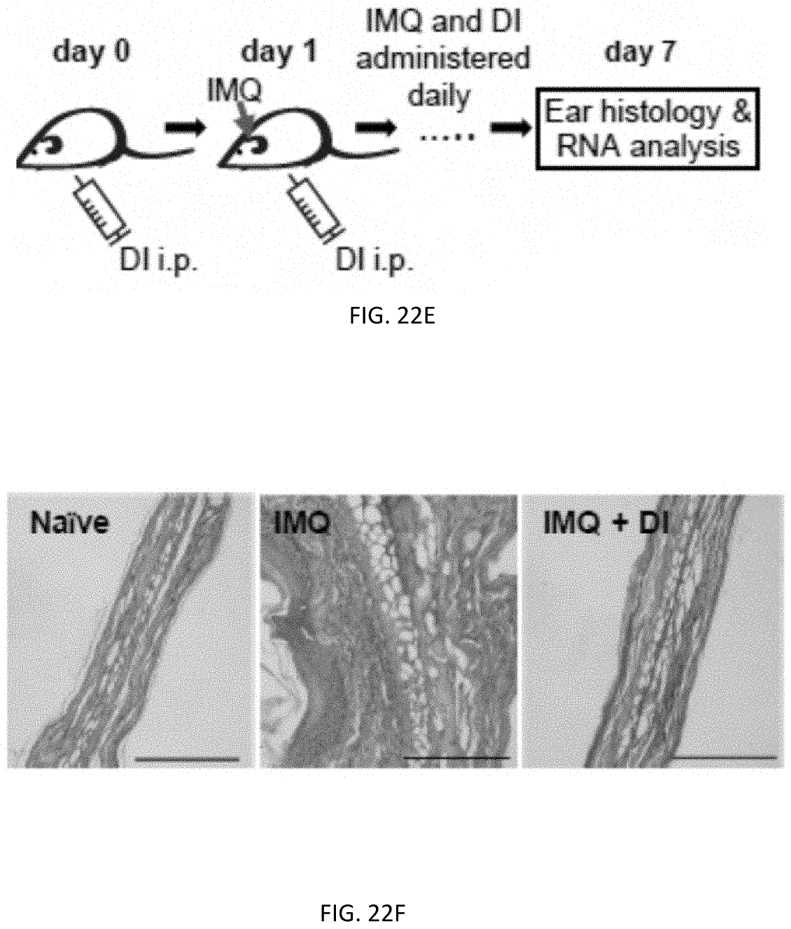

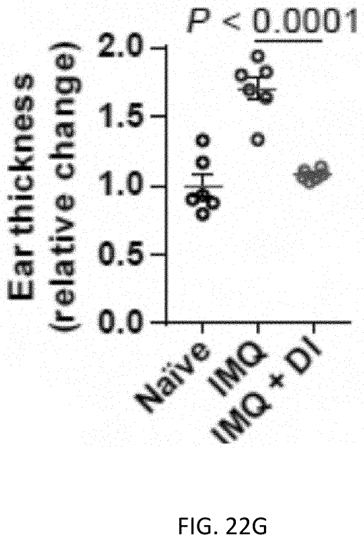

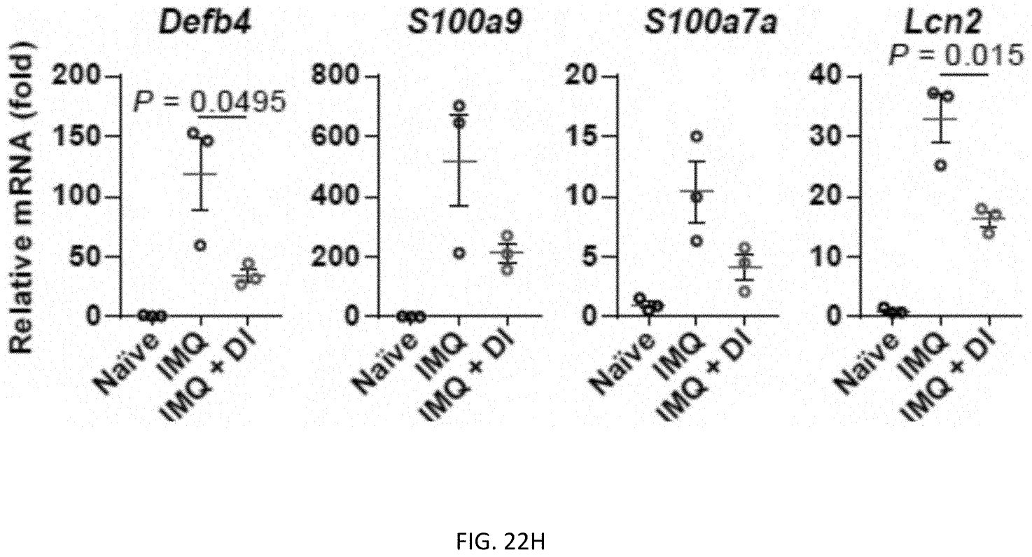

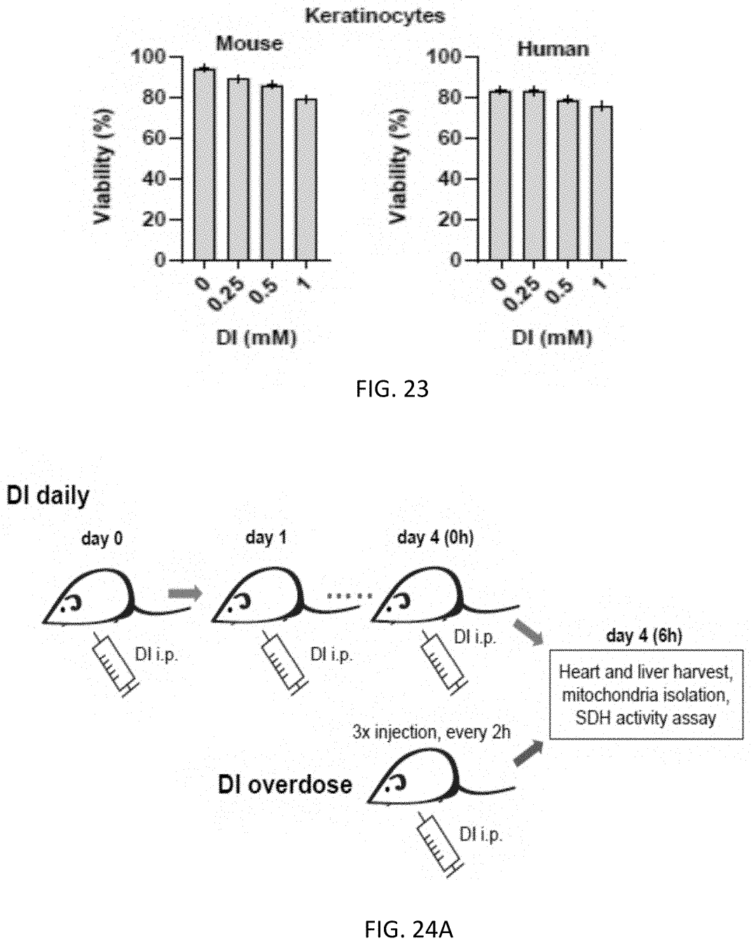

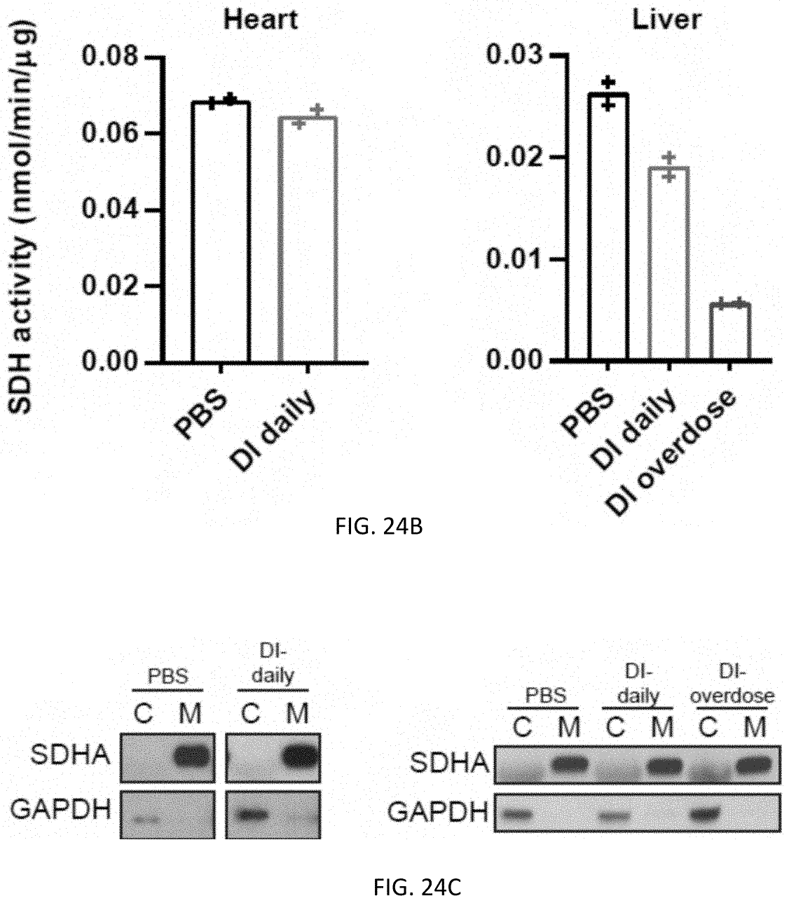

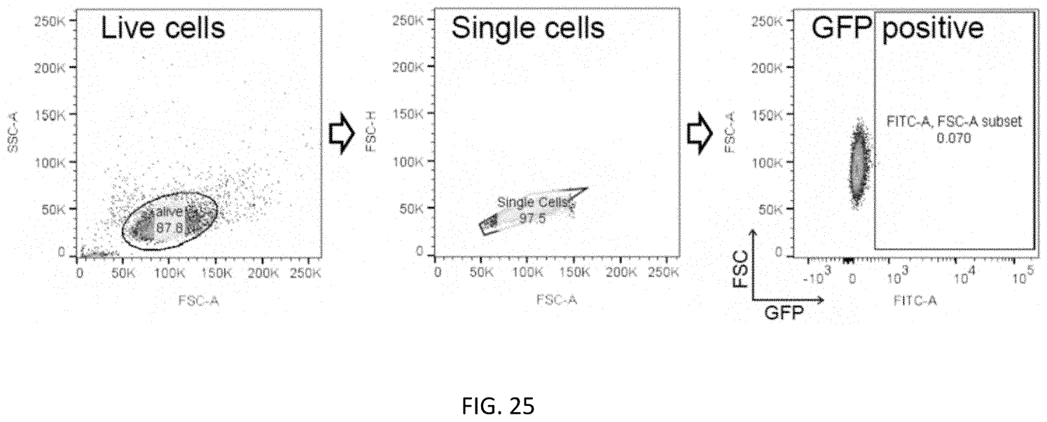

Representative of 2 cultures. (FIG. 17K) Western blot of IκBζ expression in BMDMs treated with DI in presence of EtGSH and stimulated with LPS for 1 h. (FIG. 17L) Western blot of IκBζ expression in human blood monocytes treated with DI in presence of EtGSH and stimulated with LPS for 1 h. (FIG. 17M) Cytokine production of WT or Nfkbiz−/− BMDMs treated with DI in presence of NAC, stimulated with LPS for 4 h, mean, n=2 cultures. Representative data in FIG. 17A from 2 experiments; FIG. 17E, FIG. 17H, FIG. 17I, and FIG. 17K are from 3 experiments; FIG. 17F and FIG. 17G are representative data of 3 donors; FIG. 17I, 2 donors. Two-tailed t test.

FIG. 18A, FIG. 18B, FIG. 18C, FIG. 18D, FIG. 18E, FIG. 18F, FIG. 18G and FIG. 18H show a series of images and graphs showing DI regulates IκBζ on post-transcriptional level. (FIG. 18A) Comparison of DI effects on IL-6, TNF-α and IκBζ on the protein and mRNA levels. Cytokine production is shown in BMDMs treated with DI or DMF and stimulated with LPS for 4 h (DI), mean, n=2 experiments, or 24 h (DMF), mean±s.e.m., n=3 experiments. Densitometric quantification of IκBζ protein and mRNA expression is shown for BMDMs treated with DI, stimulated with LPS for 1 h, mean, n=3 experiments, mRNA representative of 2 experiments. (FIG. 18B) Western blot of IκBζ expression in BMDMs treated with DI and stimulated with LPS for 1 h. MG132 or bafilomycin A (BafA) were added 30 min before LPS stimulation. (FIG. 18C) Nfkbiz 3′UTR reporter expressing GFP in BV2 cells treated with DI (250 μM) for 12 h and stimulated with LPS for 1 h. EMPTY vector expressing GFP only. GFP expression determined by flow cytometry. (FIG. 18D) Western blot of phosphorylated and total eIF2α in DI treated BMDMs. (FIG. 18E) Western blot of nascent protein synthesis detected using biotin-alkyne Click chemistry in BMDMs treated with DI and stimulated with LPS for 1 h. Same membrane was reprobed for IκBζ. Representative of 2 experiments. (FIG. 18F) Densitometric quantification of biotin signal in membrane in FIG. 18E. (FIG. 18G) Log fold change of proteomic signal in unstimulated versus LPS stimulated cells. (FIG. 18H) Log fold change of transcript versus protein. (FIG. 18B, FIG. 18C, and FIG. 18D) Representative data from 3 experiments.

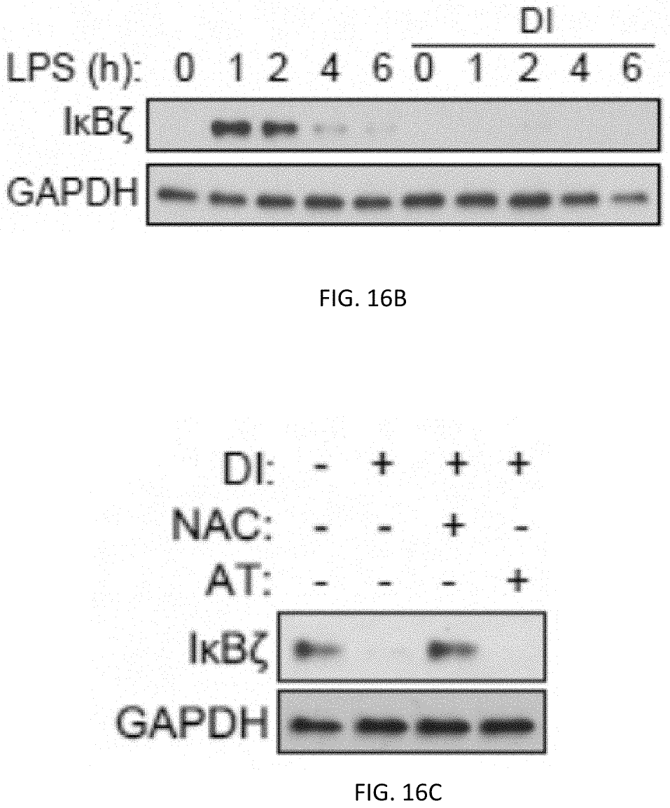

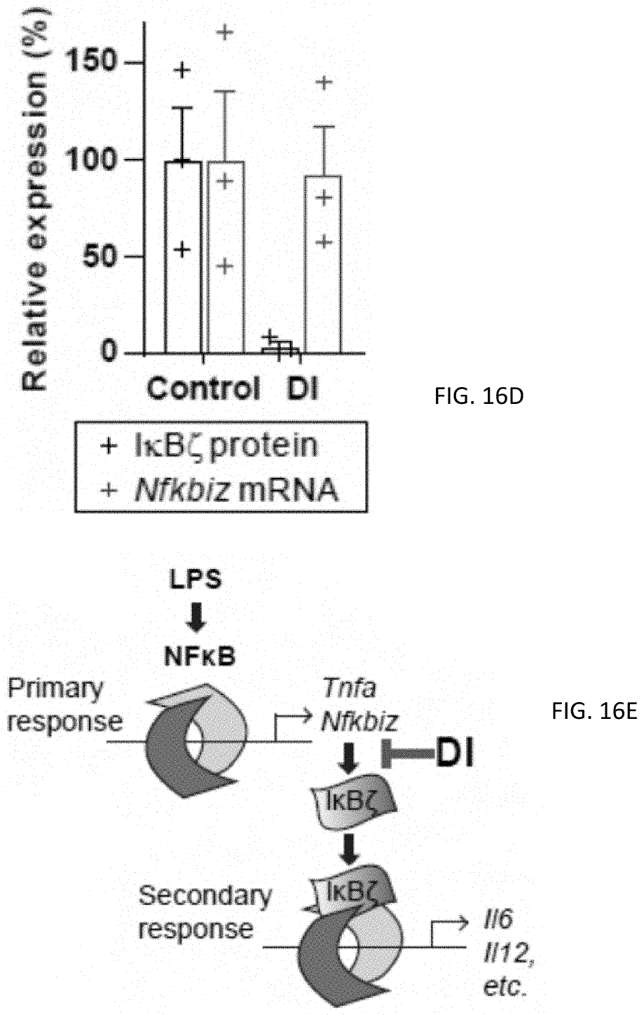

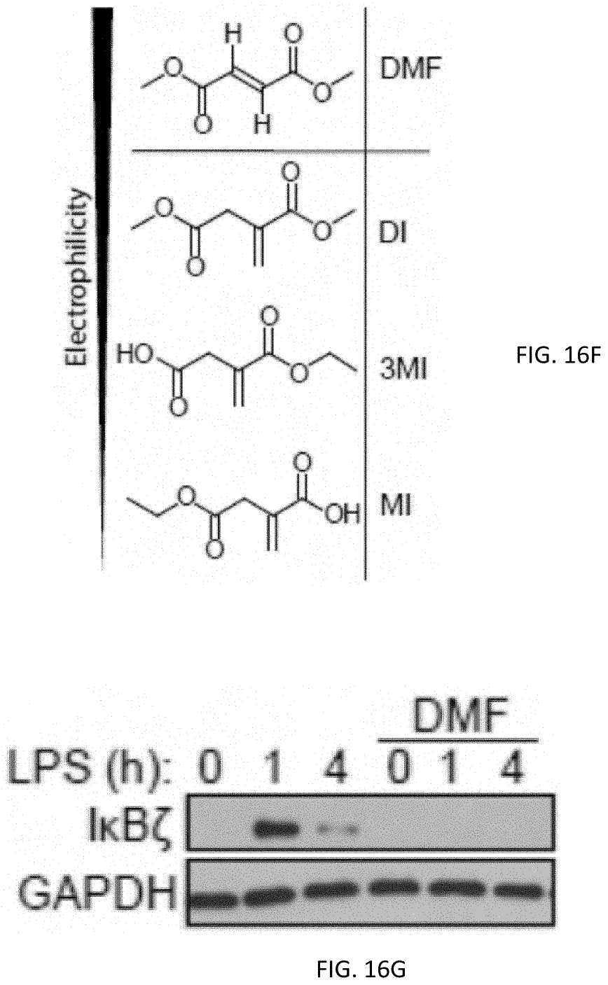

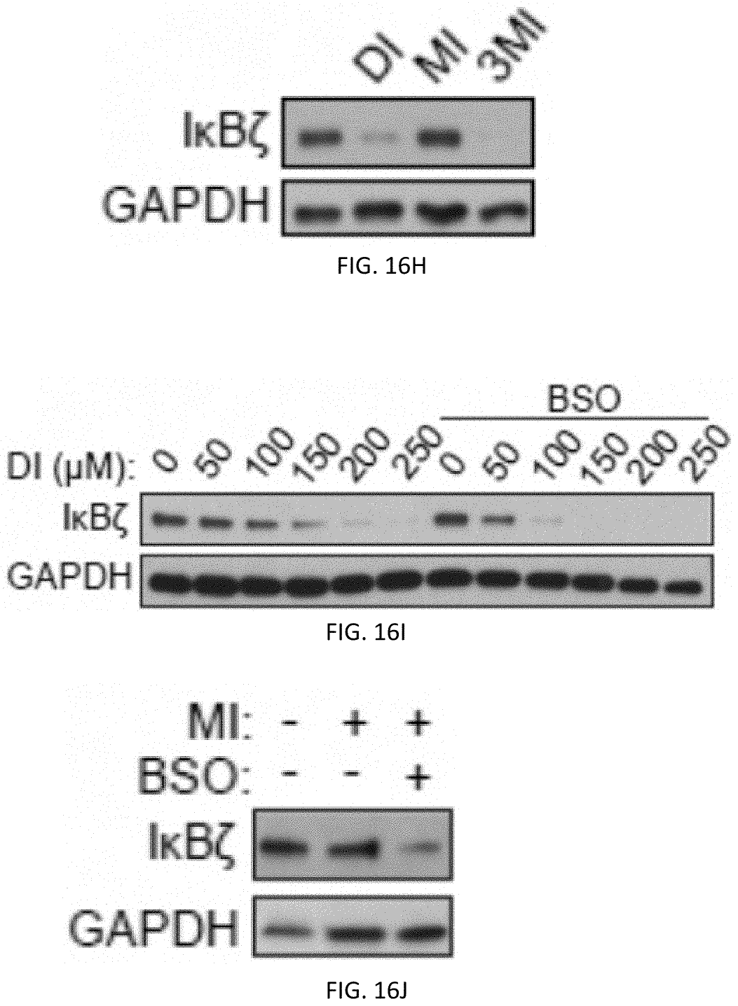

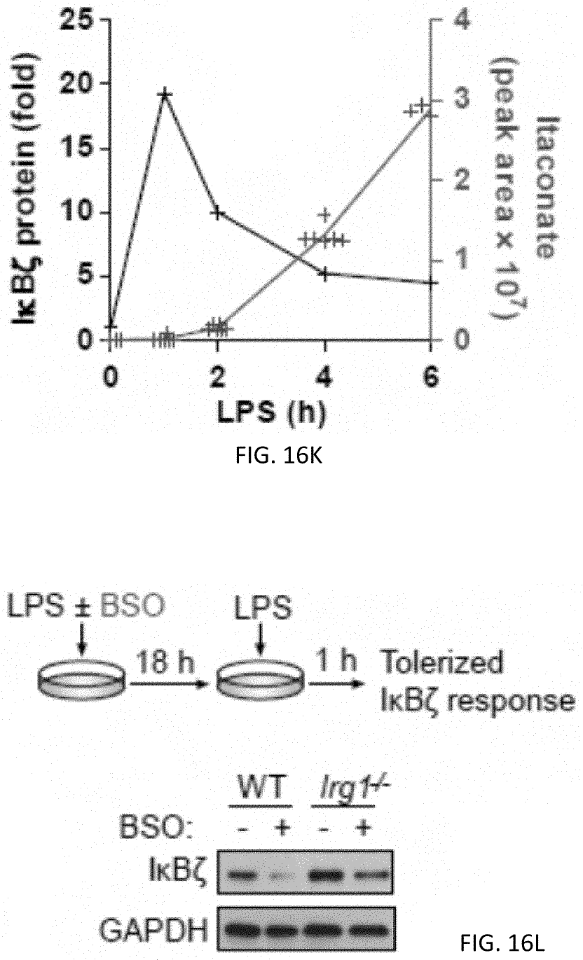

FIG. 19A, FIG. 19B, FIG. 19C, FIG. 19D, FIG. 19E and FIG. 19F show a series of images and graphs showing BSO potentiates inhibitory effect of DI. (FIG. 19A) Western blot of Nrf2 expression in BMDMs were treated with BSO or DI. (FIG. 19B) GSH levels in BMDMs treated with BSO and stimulated with LPS, mean±s.e.m., n=3 cultures. (FIG. 19C) Cytokine production in BMDMs treated with BSO and stimulated with LPS, mean±s.e.m., n=3 experiments. (FIG. 19D) Cytokine production in BMDMs treated with DI and BSO and stimulated with LPS for 4 h, mean±s.e.m., n=3 experiments. (FIG. 19E) Cytokine production in BMDMs treated with MI (10 mM) and BSO and stimulated with LPS for 4 h, mean±s.e.m., n=3 experiments. (FIG. 19F) Western blot of IκBζ expression in BMDMs tolerized with LPS in presence of BSO for 18 h and restimulated for 1 h (see e.g., FIG. 16L), asterisk shows different exposure. Western blot data are representative of 3 experiments. Two-tailed t test.

FIG. 20A, FIG. 20B, FIG. 20C, FIG. 20D, FIG. 20E, FIG. 20F, FIG. 20G and FIG. 20H show a series of images and graphs showing that DI induces Nrf2-independent response and inhibits IL-6/IκBζ axis via ATF3. (FIG. 20A and FIG. 20F) Western blot of IκBζ expression; (FIG. 20B and FIG. 20G) cytokines in BMDMs treated with DI, stimulated with LPS (FIG. 20B, 4h; FIG. 20G, 24h), mean±s.e.m., n=3 experiments. (FIG. 20C) Genes regulated by DI independently of Nrf2. (FIG. 20D) Transcriptional comparison of Atf3−/− and WT BMDMs and enrichment of DI signature. (FIG. 20E and FIG. 20H) Western blot of ATF3 expression in BMDMs (FIG. 20E) DI-treated; (FIG. 20H) tolerized in presence of BSO. Western blot data are representative of 3 experiments. Two-tailed t-test.

FIG. 21A, FIG. 21B, FIG. 21C, FIG. 21D, FIG. 21E, FIG. 21F, FIG. 21G and FIG. 21H show a series of images and graphs showing the Nrf2-independent action of DI. (FIG. 21A) Western blot of IκBζ expression in WT or Nrf2−/− BMDMs treated with DI and stimulated with LPS for 1 h. (FIG. 21B) Western blot of p62 and HO-1 in WT or Nrf2−/− BMDMs treated with DI and stimulated with LPS. (FIG. 21C) Western blot of IκBζ expression in WT and p62-deficient BMDMs treated with DI and stimulated with LPS. (FIG. 21D) Western blot of IκBζ expression in WT and Hmox1-deficient BMDMs treated with DI and stimulated with LPS. (FIG. 21E) Transcriptional comparison of Nrf2−/− and WT BMDMs treated with DI and GSEA statistics for unfolded protein response (UPR) and IFN-α pathways. (FIG. 21F) Pathways regulated by DI in Nrf2−/− independent manner. Gene ranks, normalized enrichment score (NES), P and adjusted P (padj) are shown. (FIG. 21G and FIG. 21H) Western blot of FIG. 21G, Nrf2 expression or FIG. 21H, phosphorylated and total eIF2α in DI-treated WT or Atf3−/− BMDMs. (FIG. 21I, FIG. 21J, and FIG. 21K) Western blot of ATF3 in cells treated with DI in combination of NAC or EtGSH and stimulated with LPS: (FIG. 21I and FIG. 21J) BMDMs; (FIG. 21K) human blood monocytes. Data in FIG. 21A, FIG. 21G, FIG. 21I, and FIG. 21J are representatives from 3 experiments, data in FIG. 21B, FIG. 21C, and FIG. 21H from 2 experiments, data in FIG. 21D were performed in one experiment and FIG. 21K are representative data from 2 donors.

FIG. 22A, FIG. 22B, FIG. 22C, FIG. 22D, FIG. 22E, FIG. 22F, FIG. 22G and FIG. 22H show a series of images and graphs showing that DI inhibits IL-17-mediated IκBζ induction in keratinocytes and ameliorates psoriatic pathology. (FIG. 22A and FIG. 22B) Western blot of IκBζ expression in DI-treated, IL-17A-stimulated primary keratinocytes. Representative of 3 mice/donors. (FIG. 22C and FIG. 22D) mRNA expression in DI-treated, IL-17A-stimulated (4 h) primary keratinocytes, mean±s.e.m, n=3 mice/donors. (FIG. 22E) DI administration in psoriasis model. (FIG. 22F) Ear histology. Bars 100 μM. Representative of 6 mice in 2 experiments. (FIG. 22G) quantification of FIG. 22F, mean±s.e.m., n=6 mice. (FIG. 22H) mRNA expression in ear tissue, mean±s.e.m., n=3 mice. Two-tailed t-test.

FIG. 23 shows set of graphs showing keratinocyte viability after DI treatment. Mouse or human primary keratinocytes were treated with DI for 12 h and viability was determined by propidium iodide staining and flow cytometry. Percentage of PI negative cells is shown. Representative of 2 mice/donors.

FIG. 24A, FIG. 24B and FIG. 24C show a series of images and graphs showing the lack of in vivo toxicity of DI. (FIG. 24A) Scheme of DI administration for analysis of SDH activity in heart and liver. (FIG. 24B) SDH activity in heart and liver of mice treated as in FIG. 24A, mean, n=2 technical replicates. Representative data from 2 mice. (FIG. 24C) Western blot of SDH and GAPDH in mitochondrial and cytoplasmic fractions from heart and liver of mice treated as in FIG. 24A. Representative of 2 mice.

FIG. 25 shows a series of flow cytometry gating. Dead cells and debris were gated out based on FSC and SSC. Next, single cells were gated based on FSC. Final dot plots indicate FCS versus GFP signal. GFP negative cells (non-transduced BV2 as shown) were used to set the gate for GFP positive cells.

### DETAILED DESCRIPTION

Provided herein are compositions comprising itaconate, malonate or derivatives thereof and methods of use. Applicants have discovered that itaconate, malonate and derivatives thereof can modulate immune response, modulate inflammatory function, and have anti-cancer or anti-tumor properties. In particular, Applicants have discovered that itaconate, malonate and derivatives thereof can selectively down-regulate Ikb-ζ induction in a Nrf2-independent manner and as such represents a therapeutic option for treatment of Ikbz/II17 associated diseases such as: 1) auto-inflammatory diseases such as psoriasis and multiple sclerosis, as well as 2) Ikbζ-dependent tumors such as ABC subtype of DLBCL.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular compound is disclosed and discussed and a number of modifications that can be made to a number of molecules of the compound are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Additional aspects of the disclosure are described below.

### (I) Compositions

One aspect of the present disclosure encompasses itaconate, malonate, and derivatives thereof. Itaconate, malonate, or derivatives thereof may be modified to improve potency, bioavailability, solubility, stability, handling properties, or a combination thereof, as compared to an unmodified version. Thus, in another aspect, a composition of the invention comprises modified itaconate, malonate, or derivatives thereof. In still another aspect, a composition of the invention comprises a prodrug of a itaconate, malonate, or derivatives thereof.

A composition of the invention may optionally comprise one or more additional drug or therapeutically active agent in addition to the itaconate, malonate, or derivatives thereof. A composition of the invention may further comprise a pharmaceutically acceptable excipient, carrier, or diluent. Further, a composition of the invention may contain preserving agents, solubilizing agents, stabilizing agents, wetting agents, emulsifiers, sweeteners, colorants, odorants, salts (substances of the present invention may themselves be provided in the form of a pharmaceutically acceptable salt), buffers, coating agents, or antioxidants.

Other aspects of the invention are described in further detail below.

### (a) Itaconate, Malonate, and Derivatives Thereof

In general, the compounds detailed herein include compounds comprising an itaconate, structure as diagrammed below. Itaconate is a non-peptide synthetic. Its chemical elements are expressed as C₅H₄O₄⁻², and its synthesis is known. For example, dry distillation of citric acid affords itaconic anhydride, which undergoes hydrolysis to itaconic acid. Itaconic acid is manufactured commercially.

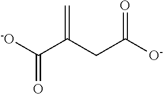

Moreover, the compounds detailed herein include compounds comprising malonate, or Propanedioate, structure as diagrammed below. Malonate is a non-peptide synthetic molecule with a molecular weight of 102.045 g/mol. Its chemical elements are expressed as C3H₂C₄⁻², and its synthesis is known. Malonic acid is manufactured commercially.

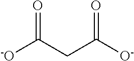

Provided herein are derivatives of itaconate and malonate. Itaconate and malonate derivatives are modified versions of itaconate or malonate, respectively that are useful as an Ikb-ζ modulation agent, an immunomodulation agent, an anti-inflammation agents, an anti-cancer agent, or an anti-tumor agent. As used herein an “itaconate derivative” or “malonate derivate” may be any derivative known in the art, a derivative of Formula (I) or Formula (II). Itaconate derivatives and malonate derivates are known in the art. In non-limiting examples, an itaconate or malonate derivative can be a dimethyl itaconate (DI), dimethyl fumarate (DMF), diethyl malonate (DEM), or a derivative thereof. See also, for example, PCT/US2017/17766 and U.S. Provisional App. No. 62/545,118, the disclosures of which are incorporated herein in their entirety by reference. Itaconate and malonate derivatives with the ability to modulate Ikb-ζ are potentially used as an immunomodulation agent, an anti-inflammation agents, an anti-cancer agent, or an anti-tumor agent.

Provided herein are compounds comprising Formula (I):

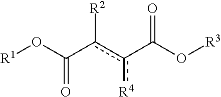

wherein:
R¹, R², R³, and R⁴ are each independently selected from the group consisting of hydrogen, deuterium, unsubstituted or substituted alkyl; unsubstituted or substituted alkenes; or unsubstituted or substituted alkynes;
   wherein
R¹, R², R³, and R⁴ can be optionally substituted with one or more groups independently selected from the group consisting of hydroxyl; C₁₋₁₀ alkyl hydroxyl; amine; C₁₋₁₀ carboxylic acid; C₁₋₁₀ carboxyl; straight chain or branched C₁₋₁₀ alkyl, optionally containing unsaturation; a C₂₋₆ cycloalkyl optionally containing unsaturation or one oxygen or nitrogen atom; straight chain or branched C₁₋₁₀ alkyl amine; heterocyclyl; heterocyclic amine; and aryl comprising a phenyl; heteroaryl containing from 1 to 4 N, O, or S atoms; unsubstituted phenyl ring; substituted phenyl ring; unsubstituted heterocyclyl; and substituted heterocyclyl, wherein
the unsubstituted phenyl ring or substituted phenyl ring can be optionally substituted with one or more groups independently selected from the group consisting of hydroxyl; C₁₋₁₀ alkyl hydroxyl; amine; C₁₋₁₀ carboxylic acid; C1-10carboxyl; straight chain or branched C₁₋₁₀ alkyl, optionally containing unsaturation; straight chain or branched C1-10alkyl amine, optionally containing unsaturation; a C₂₋₆ cycloalkyl optionally containing unsaturation or one oxygen or nitrogen atom; straight chain or branched C₁₋₁₀ alkyl amine; heterocyclyl; heterocyclic amine; aryl comprising a phenyl; and heteroaryl containing from 1 to 4 N, O, or S atoms; and
the unsubstituted heterocyclyl or substituted heterocyclyl can be optionally substituted with one or more groups independently selected from the group consisting of hydroxyl; C₁₋₁₀ alkyl hydroxyl; amine; C₁₋₁₀ carboxylic acid; C₁₋₁₀ carboxyl; straight chain or branched C₁₋₁₀ alkyl, optionally containing unsaturation; straight chain or branched C₁₋₁₀ alkyl amine, optionally containing unsaturation; a C₂₋₆ cycloalkyl optionally containing unsaturation or one oxygen or nitrogen atom; heterocyclyl; straight chain or branched C₁₋₁₀ alkyl amine; heterocyclic amine; and aryl comprising a phenyl; and heteroaryl containing from 1 to 4 N, O, or S atoms.

The dashed lines can indicate a bond, a double bond, or a delocalized bond.

In an embodiment, a compound of Formula (I) comprises any of the preceding compounds of Formula (I), wherein R¹ may be selected from the group consisting of hydrogen, deuterium, C₁₋₁₀ alkyl or CH₃. In a particular embodiment, a compound of Formula (I) comprises any of the proceeding compounds of Formula (I), wherein R¹ is H. In another particular embodiment, a compound of Formula (I) comprises any of the proceeding compounds of Formula (I), wherein R₁ is a C₁₋₁₀ alkyl. In still another particular embodiment, a compound of Formula (I) comprises any of the proceeding compounds of Formula (I), wherein R¹ is a CH₃. In still yet another particular embodiment, a compound of Formula (I) comprises any of the proceeding compounds of Formula (I), wherein R¹ is a C₂H₅.

In another embodiment, a compound of Formula (I) comprises any of the preceding compounds of Formula (I), wherein R² may be selected from the group consisting of hydrogen, deuterium CH₂, C₁₋₁₀ alkyl, or CH₃. In a particular embodiment, a compound of Formula (I) comprises any of the proceeding compounds of Formula (I), wherein R² is hydrogen. In another particular embodiment, a compound of Formula (I) comprises any of the proceeding compounds of Formula (I), wherein R² is a C₁₋₁₀ alkyl. In still another particular embodiment, a compound of Formula (I) comprises any of the proceeding compounds of Formula (I), wherein R² is a CH₃. In still yet another particular embodiment, a compound of Formula (I) comprises any of the proceeding compounds of Formula (I), wherein R² is a CH₂.

In still another embodiment, a compound of Formula (I) comprises any of the preceding compounds of Formula (I), wherein R³ may be selected from the group consisting of hydrogen, deuterium C₁₋₁₀ alkyl, CH₃, or C₈H₁₇. In a particular embodiment, a compound of Formula (I) comprises any of the proceeding compounds of Formula (I), wherein R₃ is selected is hydrogen. In another particular embodiment, a compound of Formula (I) comprises any of the proceeding compounds of Formula (I), wherein R³ is a C₁₋₁₀ alkyl. In still another particular embodiment, a compound of Formula (I) comprises any of the proceeding compounds of Formula (I), wherein R³ is a CH₃. In still yet another particular embodiment, a compound of Formula (I) comprises any of the proceeding compounds of Formula (I), wherein R³ is C₈H₁₇.

In an embodiment, a compound of Formula (I) comprises any of the preceding compounds of Formula (I), wherein R⁴ may be selected from the group consisting of hydrogen, deuterium, C₁₋₁₀ alkyl, CH₂ or CH₃. In a particular embodiment, a compound of Formula (I) comprises any of the proceeding compounds of Formula (I), wherein R⁴ is hydrogen. In another particular embodiment, a compound of Formula (I) comprises any of the proceeding compounds of Formula (I), wherein R⁴ is a C₁₋₁₀ alkyl. In still another particular embodiment, a compound of Formula (I) comprises any of the proceeding compounds of Formula (I), wherein R⁴ is CH₃. In still yet another particular embodiment, a compound of Formula (I) comprises any of the proceeding compounds of Formula (I), wherein R⁴ is a CH₂.

Provided herein are compounds comprising Formula (II):

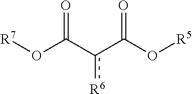

wherein:
R⁵, R⁶, and R⁷, are each independently selected from the group consisting of hydrogen, deuterium, unsubstituted or substituted alkyl; unsubstituted or substituted alkenes; or unsubstituted or substituted alkynes;
wherein
R⁵, R⁶, and R⁷, can be optionally substituted with one or more groups independently selected from the group consisting of hydroxyl; C₁₋₁₀ alkyl hydroxyl; amine; C₁₋₁₀ carboxylic acid; C₁₋₁₀ carboxyl; straight chain or branched C1-10 alkyl, optionally containing unsaturation; a C₂₋₆ cycloalkyl optionally containing unsaturation or one oxygen or nitrogen atom; straight chain or branched C₁₋₁₀ alkyl amine; heterocyclyl; heterocyclic amine; and aryl comprising a phenyl; heteroaryl containing from 1 to 4 N, O, or S atoms; unsubstituted phenyl ring; substituted phenyl ring; unsubstituted heterocyclyl; and substituted heterocyclyl, wherein
the unsubstituted phenyl ring or substituted phenyl ring can be optionally substituted with one or more groups independently selected from the group consisting of hydroxyl; C₁₋₁₀ alkyl hydroxyl; amine; C₁₋₁₀ carboxylic acid; C1-10carboxyl; straight chain or branched C₁₋₁₀ alkyl, optionally containing unsaturation; straight chain or branched C1-10alkyl amine, optionally containing unsaturation; a C₂₋₆ cycloalkyl optionally containing unsaturation or one oxygen or nitrogen atom; straight chain or branched C₁₋₁₀ alkyl amine; heterocyclyl; heterocyclic amine; aryl comprising a phenyl; and heteroaryl containing from 1 to 4 N, O, or S atoms; and
the unsubstituted heterocyclyl or substituted heterocyclyl can be optionally substituted with one or more groups independently selected from the group consisting of hydroxyl; C₁₋₁₀ alkyl hydroxyl; amine; C₁₋₁₀ carboxylic acid; C₁₋₁₀ carboxyl; straight chain or branched C₁₋₁₀ alkyl, optionally containing unsaturation; straight chain or branched C₁₋₁₀ alkyl amine, optionally containing unsaturation; a C₂₋₆ cycloalkyl optionally containing unsaturation or one oxygen or nitrogen atom; heterocyclyl; straight chain or branched C₁₋₁₀ alkyl amine; heterocyclic amine; and aryl comprising a phenyl; and heteroaryl containing from 1 to 4 N, O, or S atoms.

The dashed lines can indicate a bond, a double bond, or a delocalized bond.

In another embodiment, a compound of Formula (II) comprises any of the preceding compounds of Formula (II), wherein R⁵ may be selected from the group consisting of hydrogen, deuterium CH₂, C₁₋₁₀ alkyl, or CH₃. In a particular embodiment, a compound of Formula (II) comprises any of the proceeding compounds of Formula (II), wherein R⁵ is hydrogen. In another particular embodiment, a compound of Formula (II) comprises any of the proceeding compounds of Formula (II), wherein R⁵ is a C₁₋₁₀ alkyl. In still another particular embodiment, a compound of Formula (II) comprises any of the proceeding compounds of Formula (II), wherein R⁵ is a CH₃.

In still another embodiment, a compound of Formula (II) comprises any of the preceding compounds of Formula (II), wherein R⁶ may be selected from the group consisting of hydrogen, deuterium C₁₋₁₀ alkyl, or CH₃. In a particular embodiment, a compound of Formula (II) comprises any of the proceeding compounds of Formula (II), wherein R⁶ is selected is hydrogen. In another particular embodiment, a compound of Formula (II) comprises any of the proceeding compounds of Formula (II), wherein R⁶ is a C₁₋₁₀ alkyl. In still another particular embodiment, a compound of Formula (II) comprises any of the proceeding compounds of Formula (II), wherein R⁶ is CH₃.

In still another embodiment, a compound of Formula (II) comprises any of the preceding compounds of Formula (II), wherein R⁷ may be selected from the group consisting of hydrogen, deuterium C₁₋₁₀ alkyl, or CH₃. In a particular embodiment, a compound of Formula (II) comprises any of the proceeding compounds of Formula (II), wherein R⁷ is selected is hydrogen. In another particular embodiment, a compound of Formula (II) comprises any of the proceeding compounds of Formula (II), wherein R⁷ is a C₁₋₁₀ alkyl. In still another particular embodiment, a compound of Formula (II) comprises any of the proceeding compounds of Formula (II), wherein R⁷ is CH₃.

In exemplary embodiments, a compound of the disclosure comprises Formula (I) or Formula (II) as shown below:

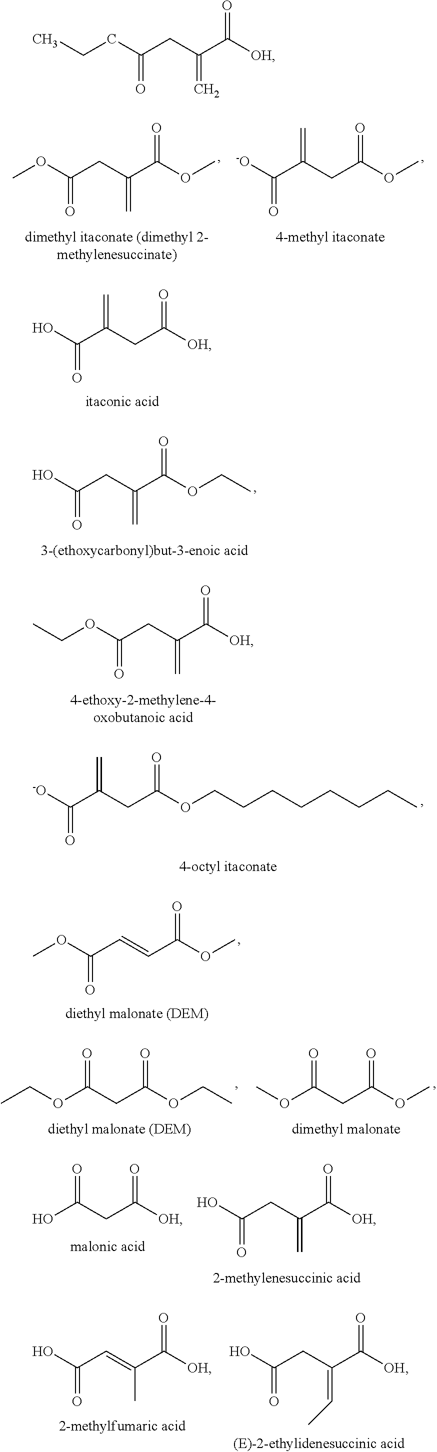

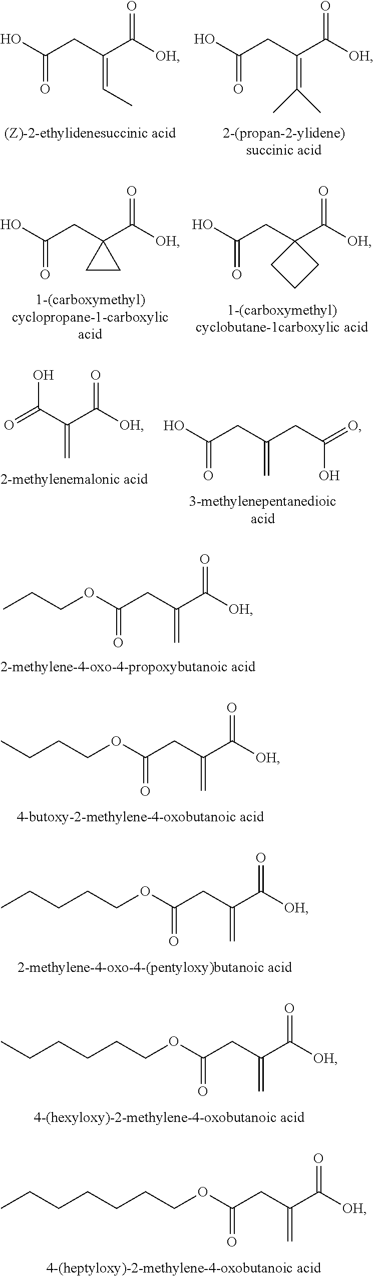

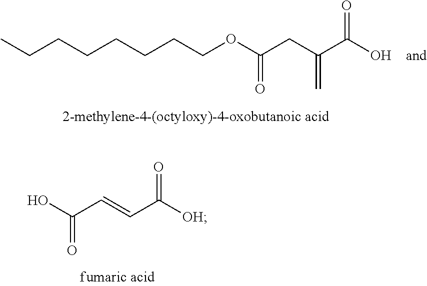 [SMILES: C=C(CC(=O)OCCCCCCCC)C(=O)O ; O=C(O)/C=C/C(=O)O]

or

a pharmaceutically acceptable salt thereof, including all tautomers and stereoisomers. In some embodiments, the itaconate, malonate, or derivative thereof is not dimethyl fumaric acid.

### (b) Components of the Composition

The present disclosure also provides pharmaceutical compositions. The pharmaceutical composition comprises itaconate, malonate, derivatives thereof, a compound of Formula (I), or a compound of Formula (II), as an active ingredient, and at least one pharmaceutically acceptable excipient.

The pharmaceutically acceptable excipient may be a diluent, a binder, a filler, a buffering agent, a pH modifying agent, a disintegrant, a dispersant, a preservative, a lubricant, taste-masking agent, a flavoring agent, or a coloring agent. The amount and types of excipients utilized to form pharmaceutical compositions may be selected according to known principles of pharmaceutical science.

In each of the embodiments described herein, a composition of the invention may optionally comprise one or more additional drug or therapeutically active agent in addition to the itaconate, malonate, derivatives thereof, a compound of Formula (I), or a compound of Formula (II). Thus, in addition to the therapies described herein, one may also provide to the subject other therapies known to be efficacious for treatment of the disease, disorder, or condition. In some embodiments, the additional drug or therapeutically active agent induces anti-inflammatory effects. In some embodiments, the secondary agent is an antibody. In some embodiments, the secondary agent is selected from a corticosteroid, a non-steroidal anti-inflammatory drug (NSAID), an intravenous immunoglobulin, a tyrosine kinase inhibitor, a fusion protein, a monoclonal antibody directed against one or more pro-inflammatory cytokines, a chemotherapeutic agent and a combination thereof. In some embodiments, the secondary agent may be a glucocorticoid, a corticosteroid, a non-steroidal anti-inflammatory drug (NSAID), a phenolic antioxidant, an anti-proliferative drug, a tyrosine kinase inhibitor, an anti-IL-5 or an IL5 receptor monoclonal antibody, an anti-IL-13 or an anti-IL-13 receptor monoclonal antibody, an IL-4 or an IL-4 receptor monoclonal antibody, an anti IgE monoclonal antibody, a monoclonal antibody directed against one or more pro-inflammatory cytokines, a TNF-α inhibitor, a fusion protein, a chemotherapeutic agent or a combination thereof. In some embodiments, the secondary agent is an anti-inflammatory drug. In some embodiments, anti-inflammatory drugs include, but are not limited to, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, curcumin, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, lysofylline, meclofenamate sodium, meclofenamic acid, meclorisone dibutyrate, mefenamic acid, mesalamine, meseclazone, methylprednisolone suleptanate, momiflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicylic acid), salicylic acid, corticosteroids, glucocorticoids, tacrolimus, pimecorlimus, mepolizumab, prodrugs thereof, and a combination thereof. In some embodiments the tyrosine kinase inhibitor is imatinib. In some embodiments the anti-IL-5 monoclonal antibody is mepolizumab or reslizumab. In some embodiments, the IL-5 receptor monoclonal antibody is benralizumab. In some embodiments, the anti-IL-13 monoclonal antibody is lebrikizumab or dulipumab. In some embodiments the anti-IL-4 monoclonal antibody is dulipumab. In some embodiments, the anti IgE monoclonal antibody is omalizumab. In some embodiments, the TNF-α inhibitor is infliximab, adalimumab, certolizumab pegol, or golimumab. In some embodiments, the secondary agent is a drug used to treat heart failure such as a beta-blocker, an ACE-inhibitor, an angiotensin receptor blocker (ARB), a neprilisine inhibitor or an aldosterone antagonist.

(i) Diluent

In one embodiment, the excipient may be a diluent. The diluent may be compressible (i.e., plastically deformable) or abrasively brittle. Non-limiting examples of suitable compressible diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, malitol, sorbitol, xylitol, maltodextrin, and trehalose. Non-limiting examples of suitable abrasively brittle diluents include dibasic calcium phosphate (anhydrous or dihydrate), calcium phosphate tribasic, calcium carbonate, and magnesium carbonate.

(ii) Binder

In another embodiment, the excipient may be a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, polypeptides, oligopeptides, and combinations thereof.

(iii) Filler

In another embodiment, the excipient may be a filler. Suitable fillers include, but are not limited to, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

(iv) Buffering Agent

In still another embodiment, the excipient may be a buffering agent. Representative examples of suitable buffering agents include, but are not limited to, phosphates, carbonates, citrates, tris buffers, and buffered saline salts (e.g., Tris buffered saline or phosphate buffered saline).

(v) pH Modifier

In various embodiments, the excipient may be a pH modifier. By way of non-limiting example, the pH modifying agent may be sodium carbonate, sodium bicarbonate, sodium citrate, citric acid, or phosphoric acid.

(vi) Disintegrant

In a further embodiment, the excipient may be a disintegrant. The disintegrant may be non-effervescent or effervescent. Suitable examples of non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. Non-limiting examples of suitable effervescent disintegrants include sodium bicarbonate in combination with citric acid and sodium bicarbonate in combination with tartaric acid.

(vii) Dispersant

In yet another embodiment, the excipient may be a dispersant or dispersing enhancing agent. Suitable dispersants may include, but are not limited to, starch, alginic acid, polyvinylpyrrolidones, guar gum, kaolin, bentonite, purified wood cellulose, sodium starch glycolate, isoamorphous silicate, and microcrystalline cellulose. (viii) Excipient

In another alternate embodiment, the excipient may be a preservative. Non-limiting examples of suitable preservatives include antioxidants, such as BHA, BHT, vitamin A, vitamin C, vitamin E, or retinyl palmitate, citric acid, sodium citrate; chelators such as EDTA or EGTA; and antimicrobials, such as parabens, chlorobutanol, or phenol.

(ix) Lubricant

In a further embodiment, the excipient may be a lubricant. Non-limiting examples of suitable lubricants include minerals such as talc or silica; and fats such as vegetable stearin, magnesium stearate, or stearic acid.

(x) Taste-Masking Agent

In yet another embodiment, the excipient may be a taste-masking agent. Taste-masking materials include cellulose ethers; polyethylene glycols; polyvinyl alcohol; polyvinyl alcohol and polyethylene glycol copolymers; monoglycerides or triglycerides; acrylic polymers; mixtures of acrylic polymers with cellulose ethers; cellulose acetate phthalate; and combinations thereof.

(xi) Flavoring Agent

In an alternate embodiment, the excipient may be a flavoring agent. Flavoring agents may be chosen from synthetic flavor oils and flavoring aromatics and/or natural oils, extracts from plants, leaves, flowers, fruits, and combinations thereof.

(xii) Coloring Agent

In still a further embodiment, the excipient may be a coloring agent. Suitable color additives include, but are not limited to, food, drug and cosmetic colors (FD&C), drug and cosmetic colors (D&C), or external drug and cosmetic colors (Ext. D&C).

The weight fraction of the excipient or combination of excipients in the composition may be about 99% or less, about 97% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

### (d) Administration

(i) Dosage Forms

The composition can be formulated into various dosage forms and administered by a number of different means that will deliver a therapeutically effective amount of the active ingredient. Such compositions can be administered orally (e.g. inhalation), parenterally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, intra-articular, or intrasternal injection, or infusion techniques. Formulation of drugs is discussed in, for example, Gennaro, A. R., Remington's Pharmaceutical Sciences, Mack Publishing Co., Easton, Pa. (18th ed, 1995), and Liberman, H. A. and Lachman, L., Eds., Pharmaceutical Dosage Forms, Marcel Dekker Inc., New York, N Y. (1980). In a specific embodiment, a composition may be a food supplement or a composition may be a cosmetic.

Solid dosage forms for oral administration include capsules, tablets, caplets, pills, powders, pellets, and granules. In such solid dosage forms, the active ingredient is ordinarily combined with one or more pharmaceutically acceptable excipients, examples of which are detailed above. Oral preparations may also be administered as aqueous suspensions, elixirs, or syrups. For these, the active ingredient may be combined with various sweetening or flavoring agents, coloring agents, and, if so desired, emulsifying and/or suspending agents, as well as diluents such as water, ethanol, glycerin, and combinations thereof. For administration by inhalation, the compounds are delivered in the form of an aerosol spray from pressured container or dispenser which contains a suitable propellant, e.g., a gas such as carbon dioxide, or a nebulizer.

For parenteral administration (including subcutaneous, intradermal, intravenous, intramuscular, intra-articular and intraperitoneal), the preparation may be an aqueous or an oil-based solution. Aqueous solutions may include a sterile diluent such as water, saline solution, a pharmaceutically acceptable polyol such as glycerol, propylene glycol, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as etheylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based solutions or suspensions may further comprise sesame, peanut, olive oil, or mineral oil. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules, and tablets.

For topical (e.g., transdermal or transmucosal) administration, penetrants appropriate to the barrier to be permeated are generally included in the preparation. Pharmaceutical compositions adapted for topical administration may be formulated as ointments, creams, suspensions, lotions, powders, solutions, pastes, gels, sprays, aerosols, or oils. In some embodiments, the pharmaceutical composition is applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administration to the eye include eye drops wherein the active ingredient is dissolved or suspended in a suitable carrier, especially an aqueous solvent. Pharmaceutical compositions adapted for topical administration in the mouth include lozenges, pastilles, and mouth washes. Transmucosal administration may be accomplished through the use of nasal sprays, aerosol sprays, tablets, or suppositories, and transdermal administration may be via ointments, salves, gels, patches, or creams as generally known in the art.

In certain embodiments, a composition comprising the itaconate, malonate, derivatives thereof, a compound of Formula (I), or a compound of Formula (II), is encapsulated in a suitable vehicle to either aid in the delivery of the compound to target cells, to increase the stability of the composition, or to minimize potential toxicity of the composition. As will be appreciated by a skilled artisan, a variety of vehicles are suitable for delivering a composition of the present invention. Non-limiting examples of suitable structured fluid delivery systems may include nanoparticles, liposomes, microemulsions, micelles, dendrimers, and other phospholipid-containing systems. Methods of incorporating compositions into delivery vehicles are known in the art.

In one alternative embodiment, a liposome delivery vehicle may be utilized. Liposomes, depending upon the embodiment, are suitable for delivery of the itaconate, malonate, derivatives thereof, a compound of Formula (I), or a compound of Formula (II), in view of their structural and chemical properties. Generally speaking, liposomes are spherical vesicles with a phospholipid bilayer membrane. The lipid bilayer of a liposome may fuse with other bilayers (e.g., the cell membrane), thus delivering the contents of the liposome to cells. In this manner, the itaconate, malonate, derivatives thereof, a compound of Formula (I), or a compound of Formula (II) may be selectively delivered to a cell by encapsulation in a liposome that fuses with the targeted cell's membrane.

Liposomes may be comprised of a variety of different types of phosolipids having varying hydrocarbon chain lengths. Phospholipids generally comprise two fatty acids linked through glycerol phosphate to one of a variety of polar groups. Suitable phospholids include phosphatidic acid (PA), phosphatidylserine (PS), phosphatidylinositol (PI), phosphatidylglycerol (PG), diphosphatidylglycerol (DPG), phosphatidylcholine (PC), and phosphatidylethanolamine (PE). The fatty acid chains comprising the phospholipids may range from about 6 to about 26 carbon atoms in length, and the lipid chains may be saturated or unsaturated. Suitable fatty acid chains include (common name presented in parentheses) n-dodecanoate (laurate), n-tretradecanoate (myristate), n-hexadecanoate (palmitate), n-octadecanoate (stearate), n-eicosanoate (arachidate), n-docosanoate (behenate), n-tetracosanoate (lignocerate), cis-9-hexadecenoate (palmitoleate), cis-9-octadecanoate (oleate), cis,cis-9,12-octadecandienoate (linoleate), all cis-9, 12, 15-octadecatrienoate (linolenate), and all cis-5,8,11,14-eicosatetraenoate (arachidonate). The two fatty acid chains of a phospholipid may be identical or different. Acceptable phospholipids include dioleoyl PS, dioleoyl PC, distearoyl PS, distearoyl PC, dimyristoyl PS, dimyristoyl PC, dipalmitoyl PG, stearoyl, oleoyl PS, palmitoyl, linolenyl PS, and the like.

The phospholipids may come from any natural source, and, as such, may comprise a mixture of phospholipids. For example, egg yolk is rich in PC, PG, and PE, soy beans contains PC, PE, PI, and PA, and animal brain or spinal cord is enriched in PS. Phospholipids may come from synthetic sources too. Mixtures of phospholipids having a varied ratio of individual phospholipids may be used. Mixtures of different phospholipids may result in liposome compositions having advantageous activity or stability of activity properties. The above mentioned phospholipids may be mixed, in optimal ratios with cationic lipids, such as N-(1-(2,3-dioleolyoxy)propyl)-N,N,N-trimethyl ammonium chloride, 1,1′-dioctadecyl-3,3,3′,3′-tetramethylindocarbocyanine perchloarate, 3,3′-deheptyloxacarbocyanine iodide, 1,1′-dedodecyl-3,3,3′,3′-tetramethylindocarbocyanine perchloarate, 1,1′-dioleyl-3,3,3′,3′-tetramethylindo carbocyanine methanesulfonate, N-4-(delinoleylaminostyryl)-N-methylpyridinium iodide, or 1,1,-dilinoleyl-3,3,3′,3′-tetramethylindocarbocyanine perchloarate.

Liposomes may optionally comprise sphingolipids, in which spingosine is the structural counterpart of glycerol and one of the one fatty acids of a phosphoglyceride, or cholesterol, a major component of animal cell membranes. Liposomes may optionally contain pegylated lipids, which are lipids covalently linked to polymers of polyethylene glycol (PEG). PEGs may range in size from about 500 to about 10,000 daltons.

Liposomes may further comprise a suitable solvent. The solvent may be an organic solvent or an inorganic solvent. Suitable solvents include, but are not limited to, dimethylsulfoxide (DMSO), methylpyrrolidone, N-methylpyrrolidone, acetronitrile, alcohols, dimethylformamide, tetrahydrofuran, or combinations thereof.

Liposomes carrying the itaconate, malonate, derivatives thereof, a compound of Formula (I), or a compound of Formula (II), may be prepared by any known method of preparing liposomes for drug delivery, such as, for example, detailed in U.S. Pat. Nos. 4,241,046; 4,394,448; 4,529,561; 4,755,388; 4,828,837; 4,925,661; 4,954,345; 4,957,735; 5,043,164; 5,064,655; 5,077,211; and 5,264,618, the disclosures of which are hereby incorporated by reference in their entirety. For example, liposomes may be prepared by sonicating lipids in an aqueous solution, solvent injection, lipid hydration, reverse evaporation, or freeze drying by repeated freezing and thawing. In a preferred embodiment the liposomes are formed by sonication. The liposomes may be multilamellar, which have many layers like an onion, or unilamellar. The liposomes may be large or small. Continued high-shear sonication tends to form smaller unilamellar lipsomes.

As would be apparent to one of ordinary skill, all of the parameters that govern liposome formation may be varied. These parameters include, but are not limited to, temperature, pH, concentration of the itaconate, malonate, derivatives thereof, a compound of Formula (I), or a compound of Formula (II), concentration and composition of lipid, concentration of multivalent cations, rate of mixing, presence of and concentration of solvent.

In another embodiment, a composition of the invention may be delivered to a cell as a microemulsion. Microemulsions are generally clear, thermodynamically stable solutions comprising an aqueous solution, a surfactant, and “oil.” The “oil” in this case, is the supercritical fluid phase. The surfactant rests at the oil-water interface. Any of a variety of surfactants are suitable for use in microemulsion formulations including those described herein or otherwise known in the art. The aqueous microdomains suitable for use in the invention generally will have characteristic structural dimensions from about 5 nm to about 100 nm. Aggregates of this size are poor scatterers of visible light and hence, these solutions are optically clear. As will be appreciated by a skilled artisan, microemulsions can and will have a multitude of different microscopic structures including sphere, rod, or disc shaped aggregates. In one embodiment, the structure may be micelles, which are the simplest microemulsion structures that are generally spherical or cylindrical objects. Micelles are like drops of oil in water, and reverse micelles are like drops of water in oil. In an alternative embodiment, the microemulsion structure is the lamellae. It comprises consecutive layers of water and oil separated by layers of surfactant. The “oil” of microemulsions optimally comprises phospholipids. Any of the phospholipids detailed above for liposomes are suitable for embodiments directed to microemulsions. The compound of the itaconate, malonate, derivatives thereof, a compound of Formula (I), or a compound of Formula (II) may be encapsulated in a microemulsion by any method generally known in the art.

In yet another embodiment, the itaconate, malonate, derivatives thereof, a compound of Formula (I), or a compound of Formula (II), may be delivered in a dendritic macromolecule, or a dendrimer. Generally speaking, a dendrimer is a branched tree-like molecule, in which each branch is an interlinked chain of molecules that divides into two new branches (molecules) after a certain length. This branching continues until the branches (molecules) become so densely packed that the canopy forms a globe. Generally, the properties of dendrimers are determined by the functional groups at their surface. For example, hydrophilic end groups, such as carboxyl groups, would typically make a water-soluble dendrimer. Alternatively, phospholipids may be incorporated in the surface of a dendrimer to facilitate absorption across the skin. Any of the phospholipids detailed for use in liposome embodiments are suitable for use in dendrimer embodiments. Any method generally known in the art may be utilized to make dendrimers and to encapsulate compositions of the invention therein. For example, dendrimers may be produced by an iterative sequence of reaction steps, in which each additional iteration leads to a higher order dendrimer. Consequently, they have a regular, highly branched 3D structure, with nearly uniform size and shape. Furthermore, the final size of a dendrimer is typically controlled by the number of iterative steps used during synthesis. A variety of dendrimer sizes are suitable for use in the invention. Generally, the size of dendrimers may range from about 1 nm to about 100 nm.

Generally, a safe and effective amount of itaconate, malonate, derivatives thereof, a compound of Formula (I), or a compound of Formula (II) is, for example, that amount that would cause the desired therapeutic effect in a subject while minimizing undesired side effects. In various embodiments, an effective amount of an itaconate, malonate, derivatives thereof, a compound of Formula (I), or a compound of Formula (II) described herein can substantially modulate IκBζ, inhibit an IκBζ/IL17 associated disease, slow the progress of an IκBζ/IL17 associated disease, or limit the development of an IκBζ/IL17 associated disease.

When used in the treatments described herein, a therapeutically effective amount of an itaconate, malonate, derivatives thereof, a compound of Formula (I), or a compound of Formula (II) can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to modulate IκBζ, inhibit an IκBζ/IL17 associated disease, slow the progress of an IκBζ/IL17 associated disease, or limit the development of an IκBζ/IL17 associated disease.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD50 (the dose lethal to 50% of the population) and the ED50, (the dose therapeutically effective in 50% of the population). The dose ratio between toxic and therapeutic effects is the therapeutic index that can be expressed as the ratio LD50/ED50, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of itaconate, malonate, derivatives thereof, a compound of Formula (I), or a compound of Formula (II) can occur as a single event or over a time course of treatment. For example, an itaconate, malonate, derivatives thereof, a compound of Formula (I), or a compound of Formula (II) can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for an IκBζ/IL17 associated disease.

Itaconate, malonate, derivatives thereof, a compound of Formula (I), or a compound of Formula (II) can be administered simultaneously or sequentially with another agent, such as an antibiotic, an anti-inflammatory, or another agent. For example, itaconate, malonate, derivatives thereof, a compound of Formula (I), or a compound of Formula (II) can be administered simultaneously with another agent, such as an antibiotic or an anti-inflammatory. Simultaneous administration can occur through administration of separate compositions, each containing one or more of itaconate, malonate, derivatives thereof, a compound of Formula (I), or a compound of Formula (II), an antibiotic, an anti-inflammatory, or another agent. Simultaneous administration can occur through administration of one composition containing two or more of itaconate, malonate, derivatives thereof, a compound of Formula (I), or a compound of Formula (II), an antibiotic, an anti-inflammatory, or another therapeutic agent for an Ikb-C associated disease (e.g., chemotherapy, radiation, or immunotherapy for cancer). Itaconate, malonate, derivatives thereof, a compound of Formula (I), or a compound of Formula (II) can be administered sequentially with an antibiotic, an anti-inflammatory, or another agent. For example, itaconate, malonate, derivatives thereof, a compound of Formula (I), or a compound of Formula (II) can be administered before or after administration of an antibiotic, an anti-inflammatory, or another agent.

### (II) Methods

The present disclosure encompasses a method of treating an IκBζ associated disease in a subject in need thereof. Generally, the method comprises administration of a therapeutically effective amount of itaconate, malonate, derivatives thereof, a compound of Formula (I), or a compound of Formula (II), so as to modulate IκBζ, inhibit an IκBζ/IL17 associated disease, slow the progress of an IκBζ/IL17 associated disease, or limit the development of an IκBζ/IL17 associated disease. In another aspect, the present disclosure encompasses a method suppressing Ikb-ζ induction in a subject in need thereof or in a biological sample, the method comprising administering to the subject, or contacting the biological sample with a composition comprising a therapeutically effective amount a compound of itaconate, malonate, derivatives thereof, a compound of Formula (I), a compound of Formula (II) or combinations thereof. In yet another aspect, the present disclosure encompasses of inhibiting tumor growth in a subject in need thereof, the method comprising administering to the subject a composition comprising a therapeutically effective amount a compound of itaconate, malonate, derivatives thereof, a compound of Formula (I), a compound of Formula (II) or combinations thereof. In still yet another aspect, the present disclosure provides a composition comprising itaconate, malonate, derivatives thereof, a compound of Formula (I), a compound of Formula (II) or combinations thereof, for use in vitro, in vivo, or ex vivo. Suitable compositions comprising itaconate, malonate, or derivatives thereof are disclosed herein, for instance those described in Section I.

According to an aspect of the invention a pharmaceutical composition comprising itaconate, malonate, derivatives thereof, a compound of Formula (I), a compound of Formula (II) or combinations thereof can treat, reduce, or prevent a disease, disorder, or condition associated with inflammation or an immune response. For example, diseases associated with inflammation or an immune response can include ischaemia-reperfusion, cardiovascular infarction, inflammatory bowel disease, psoriasis, multiple sclerosis, rheumatoid arthritis auto-inflammatory disease, or an autoimmune disease. As another example, the disease, disorder, or condition can be ischaemia-reperfusion in the heart, kidney, or brain or a tissue injury caused by ischaemia-reperfusion in the heart, kidney, or brain, or myocardial injury, where the tissue injury can occur during reperfusion.

The itaconate, malonate and derivatives thereof as described herein can treat a disease, disorder, or condition associated with inflammation or an immune response by modulating cytokines and inflammatory markers. For example, the compositions of the disclosure have been shown to modulate the expression or secretion of Casp1, iNOS, HIF-1α, pro-IL-1β, ASC, NLRP3, NOS2, iNOS, IL6, IL12B, IFNB1, IL-12p70, IL-6, IL-1β, IL-12β, NO, GM-CSF, IL-17, or IL-18. As another example, the compositions as described herein can treat a disease, disorder, or condition associated with increased expression or secretion of Casp1, iNOS, HIF-1α, pro-IL-1β, ASC, NLRP3, NOS2, iNOS, IL6, IL12B, IFNB1, IL-12p70, IL-6, IL-1β, IL-12β, NO, GM-CSF, IL-17, or IL-18.

Diseases, disorders, and conditions that can be treated by the compounds of the disclosure include: adult and juvenile Still disease; asthma; allergy; Alzheimer's disease; age-related macular degeneration; antisynthetase syndrome; autoinflammatory disease; autoimmune disease; autoimmune response; Behget disease; Blau syndrome; cancer; cardiovascular infarction; chronic infantile neurological cutaneous and articular (CINCA) syndrome; chronic recurrent multifocal osteomyelitis; cinca syndrome; classic autoinflammatory diseases; cryopyrin-associated autoinflammatory syndromes (CAPS); deficiency in IL-1 receptor antagonist (DIRA); diabetes mellitus; Erdheim-Chester syndrome (histiocytosis); extrapulmonary tuberculosis; familial atypical mycobacteriosis; familial cold autoinflammatory syndrome (FCAS); gastric cancer Risk after H. pylori Infection; Guillain-Barré syndrome; Hashimoto's thyroiditis; heart failure; hepatic fibrosis; Huntington's disease; hyper IgD syndrome (HIDS); hypoxia; ischaemia-reperfusion; immunodeficiency 29; inflammation; inflammation by HIV; inflammatory bowel disease (IBD); macrophage activation syndrome (MAS); mycobacteriosis; Miller-Fisher syndrome; Muckle-Wells syndrome (MWS); multiple sclerosis (MS); neonatal-onset multisystem inflammatory disease (NOMID); neuropathic pain; N syndrome; osteoarthritis; osteoporosis; Periodontal Disease; periodic fever, aphthous stomatitis, pharyngitis, adenitis syndrome (PFAPA); postmyocardial infarction heart failure; psoriasis; recurrent idiopathic pericarditis; recurrent pericarditis; relapsing chondritis; relapsing-remitting multiple sclerosis; rheumatoid arthritis (RA); Sapho Syndrome; Schnitzler syndrome; secondary progressive multiple sclerosis; septic shock; smoldering myeloma; Sweet syndrome; synovitis, acne, pustulosis, hyperostosis, osteitis (SAPHO); systemic juvenile rheumatoid arthritis; familial Mediterranean fever (FMF); pyogenic arthritis; pyoderma gangrenosum, acne (PAPA); TNF receptor-associated periodic syndrome (TRAPS); type 2 diabetes; urate crystal arthritis (gout); or urticarial vasculitis.

Cytokines are considered to be in a broad and loose category of small proteins (5-20 kDa) that are important in cell signaling. Their release has an effect on the behavior of cells around them. It can be said that cytokines are involved in autocrine signaling, paracrine signaling and endocrine signaling as immunomodulating agents. Their definite distinction from hormones is still part of ongoing research. Cytokines are generally known to include chemokines, interferons, interleukins, lymphokines, and tumor necrosis factors but generally not hormones or growth factors (despite some overlap in the terminology). Cytokines can be produced by a broad range of cells, including immune cells like macrophages, B lymphocytes, T lymphocytes and mast cells, as well as endothelial cells, fibroblasts, and various stromal cells; a given cytokine may be produced by more than one type of cell.

Cytokines can act through receptors, and are especially important in the immune system. Cytokines can modulate the balance between humoral and cell-based immune responses, and they can regulate the maturation, growth, or responsiveness of particular cell populations. Some cytokines can enhance or inhibit the action of other cytokines in complex ways.

Cytokines are different from hormones, which can also be important in cell signaling molecules, in that hormones circulate in less variable concentrations and hormones tend to be made by specific kinds of cells.

Cytokines can be important in health and disease, specifically in host responses to infection, immune responses, inflammation, trauma, sepsis, cancer, or reproduction.

The compositions as described herein can treat a disease, disorder, or condition associated with increased expression or secretion of Casp1, iNOS, HIF-1α, pro-IL-1β, ASC, NLRP3, NF-kappa-B, NOS2, iNOS, IL6, IL12B, IFNB1, IL-12p70, IL-6, IL-1β, IL-12β, NO, GM-CSF, IL-17, or IL-18.

(a) Ikb-ζ/IL17 and NF-κβ Associated Diseases

Interleukin-17 (IL-17) family cytokines have recently emerged as important players in inflammatory responses. IL-17A and IL-17F, which are most highly related among IL-17 family cytokines, are expressed by a distinct T cell subset, Th17 cells. IκBζ was identified to be induced by STAT3 and promote Th17 cell differentiation.

NF-kappa-B inhibitor zeta (aka, NFKBIZ, IKBZ, INAP, MAIL, NFKB inhibitor zeta) is a protein that in humans is encoded by the NFKBIZ gene. This gene is a member of the Ankyrin-repeat family and is induced by lipopolysaccharide (LPS). The C-terminal portion of the encoded product which contains the Ankyrin repeats, shares high sequence similarity with the I kappa B family of proteins. The latter are known to play a role in inflammatory responses to LPS by their interaction with NF-B proteins through Ankyrin-repeat domains.

NF-κB associated inflammatory diseases can include rheumatoid arthritis (RA), atherosclerosis, multiple sclerosis, chronic inflammatory demyelinating polyradiculoneuritis, asthma, inflammatory bowel disease, Heliobacter pylori-associated gastritis, or systemic inflammatory response syndrome (Tak et al. J Clin Invest. 2001 Jan. 1; 107(1): 7-11).

NF-κB associated cancer can include prostate cancer, breast cancer, lung cancer, head and neck squamous cell carcinomas, glioblastoma, skin cancer, brain cancer, glioma, liver cancer, non-small cell lung cancers (NSCLC), lymphoma, leukemia, rectal cancer, gastric cancer, or colon cancer (Xia et al. Cancer Immunol Res. 2014 September; 2(9): 823-830).

An Ikbζ/IL17 associated disease can be psoriasis, multiple sclerosis, or activated B-cell-like (ABC) subtype of diffuse large B-cell lymphoma (DLBCL), but not to GCB subtype. ABC DLBCL is associated with substantially worse outcomes when treated with standard chemoimmunotherapy. The ABC subtype is characterized by chronic active B-cell receptor (BCR) signaling, which stimulates NF-κB activity.

Other Ikbζ/IL17 associated diseases can be a Ikbζ/IL17 associated carcinoma, an autoimmune disease, Sjögren's syndrome (or a Sjögren's syndrome-like autoimmune disease), lupus, myxoid liposarcoma, brain glioblastoma multiforme, hypersensitivity syndrome, multiple sclerosis, susceptibility to pneumococcal disease, ocular surface inflammatory disorders, epilepsy, or meningococcal meningitis, atopic dermatitis, bursitis, tendinitis, psoriasis, or allergic conjunctivitis.

Interleukin 17 is a pro-inflammatory cytokine produced by T-helper cells, gamma-delta T cells and subsets of innate lymphoid cells (Sutton et al, EJI 2012; Klose and Artis, Nat Immunol, 2016), and is induced and/or promoted by cytokines including IL-6, IL-23, IL-1β, or TGFβ. To elicit its functions, IL-17 binds to a type I cell surface receptor called IL-17R of which there are at least three variants IL17RA, IL17RB, and IL17RC. IL-17 acts as a potent mediator in delayed-type reactions by increasing chemokine production in various tissues. Signaling from IL-17 recruits monocytes and neutrophils to the site of inflammation in response to invasion by pathogens, similar to Interferon gamma. In promoting inflammation, IL-17 has been demonstrated to act synergistically with tumor necrosis factor and interleukin-1. This activity can also be redirected towards the host and result in various autoimmune disorders that involve chronic inflammation, such as the skin disorder psoriasis.

IL-17 is implicated in numerous inflammatory diseases (see e.g., psoriasis, vitiligo, allergies, autoimmune disease, rheumatoid arthritis, inflammatory bowel disease, multiple sclerosis, or asthma) (Wang et al PlosOne 2011).

IL-17 has also been associated with cancer. For example, an IL-17 associated cancer can be breast cancer, lung cancer, colorectal cancer (CRC), prostate cancer, breast cancer, myeloma, melanoma, ovarian cancer, renal cell carcinoma, colon cancer, acute myeloid leukemia, gastric cancer, lymphoma, pancreatic cancer, or lung cancer (Murugaiyan et al. J Immunol 2009; 183:4169-4175)

As described herein, itaconate and derivatives thereof can down-regulate Ikbζ induction. The inventors have shown the DI-effect on Ikb-ζ occurs in Nrf2-independent manner. Ikb-ζ is an inducible nuclear protein that regulates Toll/IL-1-receptor-mediated gene expression and has been shown to be critical for production of IL6 but not TNFα in macrophages4. The inventors have also shown, itaconate and derivatives thereof can down-regulate IL17-induced Ikbζ activation. Furthermore, itaconate and derivatives thereof have been shown to inhibit STAT3 activation. DI inhibits STAT3 activation in macrophages by mechanism that does not involve Nrf2.

(b) IL-1β Associated Disease

Interleukin 1 beta (IL1β), including pro-IL-1β, also known as leukocytic pyrogen, leukocytic endogenous mediator, mononuclear cell factor, lymphocyte activating factor or other names, is a cytokine protein that in humans is encoded by the IL1B gene. There are two genes for interleukin-1 (IL-1): IL-1 alpha and IL-1 beta. IL-1β precursor is cleaved by cytosolic caspase 1 (interleukin 1 beta convertase) to form mature IL-1β.

Increased production of IL-1β can causes a number of different autoinflammatory syndromes, most notably the monogenic conditions referred to as Cryopyrin-Associated Autoinflammatory Syndromes (CAPS), due to mutations in the inflammasome receptor NLRP3 which triggers processing of IL-1β.

IL-1β can be associated with a number of autoinflammatory diseases. For these, neutralization of IL-1β results in a rapid and sustained reduction in disease severity. Treatment for autoimmune diseases often includes immunosuppressive drugs whereas neutralization of IL-1β is mostly anti-inflammatory.

For example IL-1β implicated diseases can include gout, type 2 diabetes, heart failure, recurrent pericarditis, rheumatoid arthritis, and smoldering myeloma also are responsive to IL-1β neutralization.

It is well established that IL-1β is implicated in numerous inflammatory diseases (see e.g., Dinarello, Blood. 2011 Apr. 7; 117(14): 3720-3732). For example, the following conditions can be treated with blocking or reduction in IL-1β: Classic autoinflammatory diseases; Familial Mediterranean fever (FMF); Pyogenic arthritis, pyoderma gangrenosum, acne (PAPA); Cryopyrin-associated periodic syndromes (CAPS); Hyper IgD syndrome (HIDS); Adult and juvenile Still disease; Schnitzler syndrome; TNF receptor-associated periodic syndrome (TRAPS); Blau syndrome; Sweet syndrome; Deficiency in IL-1 receptor antagonist (DIRA); Recurrent idiopathic pericarditis; Macrophage activation syndrome (MAS); Urticarial vasculitis; Antisynthetase syndrome; Relapsing chondritis; Behget disease; Erdheim-Chester syndrome (histiocytosis); Synovitis, acne, pustulosis, hyperostosis, osteitis (SAPHO); Rheumatoid arthritis; Periodic fever, aphthous stomatitis, pharyngitis, adenitis syndrome (PFAPA); Urate crystal arthritis (gout); Type 2 diabetes; Smoldering multiple myeloma; Postmyocardial infarction heart failure; or Osteoarthritis.

Diseases associated with IL-1β include Gastric Cancer Risk After H. pylori Infection and Periodontal Disease.

(c) IL-17 Associated Diseases

Interleukin 17 is a pro-inflammatory cytokine produced by T-helper cells, gamma-delta T cells and subsets of innate lymphoid cells (Sutton et al, EJI 2012; Klose and Artis, Nat Immunol, 2016), and is induced and/or promoted by cytokines including IL-6, IL-23, IL-1β, or TGFβ. To elicit its functions, IL-17 binds to a type I cell surface receptor called IL-17R of which there are at least three variants IL17RA, IL17RB, and IL17RC. IL-17 acts as a potent mediator in delayed-type reactions by increasing chemokine production in various tissues. Signaling from IL-17 recruits monocytes and neutrophils to the site of inflammation in response to invasion by pathogens, similar to Interferon gamma. In promoting inflammation, IL-17 has been demonstrated to act synergistically with tumor necrosis factor and interleukin-1. This activity can also be redirected towards the host and result in various autoimmune disorders that involve chronic inflammation, such as the skin disorder psoriasis.

IL-17 is implicated in numerous inflammatory diseases (see e.g., psoriasis, vitiligo, allergies, autoimmune disease, rheumatoid arthritis, inflammatory bowel disease, multiple sclerosis, or asthma) (Wang et al PlosOne 2011).

(d) IL18 Associated Diseases

IL-18 has been shown to induce severe inflammatory reactions, which suggests its role in certain inflammatory disorders. For example, IL-18 has been implicated in age-related macular degeneration, Hashimoto's thyroiditis, Alzheimer's disease.

Diseases associated with IL18 include Adult-Onset Still's Disease and Sapho Syndrome.

(e) Casp1 Associated Diseases

Caspase-1/Interleukin-1 converting enzyme (ICE) plays a central role in cell immunity as an inflammatory response initiator. Caspase-1 has also been shown to induce necrosis and may also function in various developmental stages. Studies suggest a role in the pathogenesis of Huntington's disease. Alternative splicing of the gene results in five transcript variants encoding distinct isoforms. Recent studies implicated caspase-1 in promoting CD4 T-cell death and inflammation by HIV, two signature events that fuel HIV disease progression to AIDS.

(f) Inducible Nitric Oxide Synthases (iNOS), NOS2, and NO Associated Diseases

Nitric oxide synthases (NOSs) are a family of enzymes catalyzing the production of nitric oxide (NO) from L-arginine. NO is an important cellular signaling molecule. It helps modulate vascular tone, insulin secretion, airway tone, and peristalsis, and is involved in angiogenesis and neural development. It may function as a retrograde neurotransmitter. Nitric oxide is mediated in mammals by the calcium-calmodulin controlled isoenzymes eNOS (endothelial NOS) and nNOS (neuronal NOS). The inducible isoform, iNOS, is involved in immune response, binds calmodulin at physiologically relevant concentrations, and produces NO as an immune defense mechanism, as NO is a free radical with an unpaired electron. It is the proximate cause of septic shock and may function in autoimmune disease.

(q) HIF-1α Associated Diseases

Hypoxia-inducible factor 1-alpha, also known as HIF-1-alpha, is a subunit of a heterodimeric transcription factor hypoxia-inducible factor 1 (HIF-1) that is encoded by the HIF1A gene. It is a basic helix-loop-helix PAS domain containing protein, and is considered as the master transcriptional regulator of cellular and developmental response to hypoxia. The dysregulation and overexpression of HIF1A by either hypoxia or genetic alternations have been heavily implicated in cancer biology, as well as a number of other pathophysiologies, specifically in areas of vascularization and angiogenesis, energy metabolism, cell survival, and tumor invasion. Two other alternative transcripts encoding different isoforms have been identified.

(h) Pycard (ASC) Associated Diseases

Apoptosis-associated speck-like protein containing a CARD or ASC is a protein that in humans is encoded by the PYCARD gene.

This gene encodes an adaptor protein that is composed of two protein-protein interaction domains: an N-terminal PYRIN-PAAD-DAPIN domain (PYD) and a C-terminal caspase-recruitment domain (CARD). The PYD and CARD domains are members of the six-helix bundle death domain-fold superfamily that mediates assembly of large signaling complexes in the inflammatory and apoptotic signaling pathways via the activation of caspase. In normal cells, this protein is localized to the cytoplasm; however, in cells undergoing apoptosis, it forms ball-like aggregates near the nuclear periphery. Two transcript variants encoding different isoforms have been found for this gene.

Diseases associated with PYCARD include Chronic Recurrent Multifocal Osteomyelitis and Cinca Syndrome.

(i) NLRP3 Associated Diseases

NACHT, LRR and PYD domains-containing protein 3 (NALP3) also known by cryopyrin is a protein that in humans is encoded by the NLRP3 gene located on the long arm of chromosome 1.

NALP3 is expressed predominantly in macrophages and as a component of the inflammasome, detects products of damaged cells such as extracellular ATP and crystalline uric acid. Activated NALP3 in turn triggers an immune response. Mutations in the NLRP3 gene are associated with a number of organ specific autoimmune diseases.

Mutations in the NLRP3 gene have been associated with a spectrum of dominantly inherited autoinflammatory diseases called cryopyrin-associated periodic syndrome (CAPS). This includes familial cold autoinflammatory syndrome (FCAS), Muckle-Wells syndrome (MWS), chronic infantile neurological cutaneous and articular (CINCA) syndrome, and neonatal-onset multisystem inflammatory disease (NOMID).

Defects in this gene have also been linked to familial Mediterranean fever. In addition, the NALP3 inflammasome has a role in the pathogenesis of gout and neuroinflammation occurring in protein-misfolding diseases, such as Alzheimer's, Parkinson's, and Prion diseases.

Deregulation of NALP3 has been connected with carcinogenesis. For example, all the components of the NALP3 inflammasome are downregulated or completely lost in human hepatocellular carcinoma.

Diseases associated with NALP3 are familial cold autoinflammatory syndrome (FCAS), Muckle-Wells syndrome (MWS), chronic infantile neurological cutaneous and articular (CINCA) syndrome, and neonatal-onset multisystem inflammatory disease (NOMID).

(i) IL-6 Associated Diseases

Interleukin 6 (IL-6) is an interleukin that acts as both a pro-inflammatory cytokine and an anti-inflammatory myokine. In humans, it is encoded by the IL6 gene.

Interleukin 6 is secreted by B cells, T cells, and macrophages to stimulate immune response, e.g. during infection and after trauma, especially burns or other tissue damage leading to inflammation. IL-6 also plays a role in fighting infection, as IL-6 has been shown in mice to be required for resistance against bacterium Streptococcus pneumoniae.

In addition, osteoblasts secrete IL-6 to stimulate osteoclast formation. Smooth muscle cells in the tunica media of many blood vessels also produce IL-6 as a pro-inflammatory cytokine. IL-6's role as an anti-inflammatory cytokine is mediated through its inhibitory effects on TNF-alpha and IL-1, and activation of IL-1ra and IL-10.

IL-6 is associated with and stimulates the inflammatory and autoimmune processes in many diseases such as diabetes, atherosclerosis, depression, Alzheimer's Disease, systemic lupus erythematosus, multiple myeloma, prostate cancer, Behget's disease, inflammatory bowel disease (Neurath, Nat Rev Immunol, 2014), rheumatoid arthritis, vitiligo, and systemic sclerosis (O'Reilly et al, Clin and Translational Immunol, 2013).

IL-6 has been associated with diabetes mellitus and systemic juvenile rheumatoid arthritis.

(k) IFNB Associated Diseases

Interferons (IFNs) are a group of signaling proteins made and released by host cells in response to the presence of several pathogens, such as viruses, bacteria, parasites, and also tumor cells. In a typical scenario, a virus-infected cell will release interferons causing nearby cells to heighten their anti-viral defenses.

IFNs belong to the large class of proteins known as cytokines, molecules used for communication between cells to trigger the protective defenses of the immune system that help eradicate pathogens. Interferons are named for their ability to “interfere” with viral replication by protecting cells from virus infections. IFNs also have various other functions: they activate immune cells, such as natural killer cells and macrophages; they increase host defenses by up-regulating antigen presentation by virtue of increasing the expression of major histocompatibility complex (MHC) antigens. Certain symptoms of infections, such as fever, muscle pain and “flu-like symptoms”, are also caused by the production of IFNs and other cytokines.

Overactivation of type I interferon secretion is linked to autoimmune diseases. Interferon beta is a protein that in humans is encoded by the IFNB1 gene. Diseases associated with IFNB1 include Relapsing-Remitting Multiple Sclerosis and Secondary Progressive Multiple Sclerosis.

(l) IL-12p70 and IL-126/IL12B Associated Diseases

Subunit beta of interleukin 12 (also known as IL-12B, natural killer cell stimulatory factor 2, cytotoxic lymphocyte maturation factor p40, or interleukin-12 subunit p40) is a protein that in humans is encoded by the IL12B gene. IL-12B is a common subunit of interleukin 12 and Interleukin 23.

This gene encodes a subunit of interleukin 12, a cytokine that acts on T and natural killer cells, and has a broad array of biological activities. Interleukin 12 is a disulfide-linked heterodimer composed of the 40 kD cytokine receptor like subunit encoded by this gene, and a 35 kD subunit encoded by IL12A. This cytokine is expressed by activated macrophages that serve as an essential inducer of Th1 cells development. This cytokine has been found to be important for sustaining a sufficient number of memory/effector Th1 cells to mediate long-term protection to an intracellular pathogen. Overexpression of this gene was observed in the central nervous system of patients with multiple sclerosis (MS), suggesting a role of this cytokine in the pathogenesis of the disease. The promoter gene polymorphism of this gene has been reported to be associated with the severity of atopic and non-atopic asthma in children.

Interleukin 12 (IL-12) is an interleukin that is naturally produced by dendritic cells, macrophages, neutrophils, and human B-lymphoblastoid cells (NC-37) in response to antigenic stimulation. IL-12 is composed of a bundle of four alpha helices. It is a heterodimeric cytokine encoded by two separate genes, IL-12A (p35) and IL-12B (p40). The active heterodimer (referred to as ‘p70’), and a homodimer of p40 are formed following protein synthesis.

IL-12 is linked with autoimmunity. Administration of IL-12 to people suffering from autoimmune diseases was shown to worsen the autoimmune phenomena. This is believed to be due to its key role in induction of Th1 immune responses. In contrast, IL-12 gene knock-out in mice or a treatment of mice with IL-12 specific antibodies ameliorated the disease.

Interleukin 12 (IL-12) is produced by activated antigen-presenting cells (dendritic cells, macrophages). It promotes the development of Th1 responses and is a powerful inducer of IFNγ production by T and NK cells.

Other diseases associated with IL12B include Immunodeficiency 29, Mycobacteriosis and Familial Atypical Mycobacteriosis.

IL-12p70 has been shown to be overexpressed in Crohn's disease.

Dysregulated expression of IL-12 p40 can lead to prolonged, unresolved inflammation manifesting into chronic inflammatory disorders such as inflammatory bowel disease (IBD).

Overexpression IL12B was observed in the central nervous system of patients with multiple sclerosis (MS).

Diseases associated with IL12RB1 include Immunodeficiency 30 and Familial Atypical Mycobacteriosis.

(m) GM-CSF Associated Diseases

Granulocyte-macrophage colony-stimulating factor (GM-CSF), also known as colony stimulating factor 2 (CSF2), is a monomeric glycoprotein secreted by macrophages, T cells, B cells, mast cells, NK cells, endothelial cells and fibroblasts that functions as a cytokine. The pharmaceutical analogs of naturally occurring GM-CSF are called sargramostim and molgramostim.

GM-CSF is found in high levels in joints with rheumatoid arthritis, in the cerebrospinal fluid of MS patients and in the serum of patients with acute aortic aneurysm. Also, its receptor is highly expressed in subsets of myeloid cells in patients with rheumatoid arthritis and psoriatic arthritis. GM-CSF can activate microglial cells that promote inflammation of the central nervous system. Targeting GM-CSF may reduce inflammation or damage and could be beneficial for patients with rheumatoid arthritis, MS, plaque psoriasis, and asthma (Wicks and Roberts, Nat Rev Rheumatology, 2016).

(n) P2rx7

P2X purinoceptor 7 is a protein that in humans is encoded by the P2RX7 gene.

The product of this gene belongs to the family of purinoceptors for ATP. Multiple alternatively spliced variants which would encode different isoforms have been identified although some fit nonsense-mediated decay criteria.

The receptor is found in the central and peripheral nervous systems, in microglia, in macrophages, in uterine endometrium, and in the retina. The P2X7 receptor also serves as a pattern recognition receptor for extracellular ATP-mediated apoptotic cell death, regulation of receptor trafficking, mast cell degranulation, and inflammation.

Diseases associated with P2RX7 include Extrapulmonary Tuberculosis and N Syndrome. Among its related pathways are Peptide ligand-binding receptors and Nucleotide-binding domain, leucine rich repeat containing receptor (NLR) signaling pathways.

Microglial P2X7 receptors are thought to be involved in neuropathic pain because blockade or deletion of P2X7 receptors results in decreased responses to pain, as demonstrated in vivo.

Moreover, P2X7 receptor signaling increases the release of pro-inflammatory molecules such as IL-1β, IL-6, and TNF-α. In addition, P2X7 receptors have been linked to increases in pro-inflammatory cytokines such as CXCL2 and CCL3. Interestingly, P2X7 receptors are also linked to P2X4 receptors, which are also associated with neuropathic pain mediated by microglia.

P2RX7 has also been linked to osteoporosis. Mutations in this gene have been associated to low lumbar spine bone mineral density and accelerated bone loss in post-menopausal women.

P2RX7 has also been linked to diabetes. The ATP/P2X7R pathway may trigger T-cell attacks on the pancreas, rendering it unable to produce insulin. This autoimmune response may be an early mechanism by which the onset of diabetes is caused.

P2RX7 has also been linked to hepatic fibrosis. One study in mice showed that blockade of P2X7 receptors attenuates onset of liver fibrosis.

(o) LPS-Mediated Immune Response Associated Diseases

The immunomodulatory agents as described herein have been shown to suppress a lipopolysaccharide (LPS)-mediated immune response. Diseases associated with LPS-mediated immune response that can be treated with immunomodulatory agents can include: autoimmune disease and responses, MS flare ups, Guillain-Barré syndrome and a variant of Guillain-Barré called Miller-Fisher syndrome.

Methods described herein are generally performed on a subject in need thereof. A subject may be a rodent, a human, a livestock animal, a companion animal, or a zoological animal. In one embodiment, the subject may be a rodent, e.g. a mouse, a rat, a guinea pig, etc. In another embodiment, the subject may be a livestock animal. Non-limiting examples of suitable livestock animals may include pigs, cows, horses, goats, sheep, llamas and alpacas. In still another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In a preferred embodiment, the subject is a human.

### III. Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to compositions and pharmaceutical formulations comprising an Ikb-ζ modulation agent. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

### Definitions

When introducing elements of the present disclosure or the preferred aspects(s) thereof, the articles “a,” “an,” “the,” and “said” are intended to mean that there are one or more of the elements. The terms “comprising,” “including,” and “having” are intended to be inclusive and mean that there may be additional elements other than the listed elements.

As used herein, the following definitions shall apply unless otherwise indicated. For purposes of this invention, the chemical elements are identified in accordance with the Periodic Table of the Elements, CAS version, and the Handbook of Chemistry and Physics, 75^{th} Ed. 1994. Additionally, general principles of organic chemistry are described in “Organic Chemistry,” Thomas Sorrell, University Science Books, Sausalito: 1999, and “March's Advanced Organic Chemistry,” 5^{th} Ed., Smith, M. B. and March, J., eds. John Wiley & Sons, New York: 2001, the entire contents of which are hereby incorporated by reference.

The term “alkyl” as used herein alone or as part of a group refers to saturated monovalent hydrocarbon radicals having straight or branched hydrocarbon chains or, in the event that at least 3 carbon atoms are present, cyclic hydrocarbons or combinations thereof and contains 1 to 20 carbon atoms (C.sub.1-20alkyl), suitably 1 to 10 carbon atoms (C.sub.1-10alkyl), preferably 1 to 8 carbon atoms (C.sub.1-8alkyl), more preferably 1 to 6 carbon atoms (C.sub.1-4alkyl), and even more preferably 1 to 4 carbon atoms (C.sub.1-4alkyl). Examples of alkyl radicals include methyl, ethyl, propyl, isopropyl, n-butyl, isobutyl, sec-butyl, tert-butyl, pentyl, isoamyl, hexyl, cyclopropyl, cyclobutyl, cyclopentyl, cyclohexyl, and the like.

The term “alkenyl” as used herein alone or as part of a group refers to monovalent hydrocarbon radicals having a straight or branched hydrocarbon chains having one or more double bonds and containing from 2 to about 18 carbon atoms, preferably from 2 to about 8 carbon atoms, more preferably from 2 to about 5 carbon atoms. Examples of suitable alkenyl radicals include ethenyl, propenyl, alkyl, 1,4-butadienyl, and the like.

The term “alkynyl” as used herein alone or as part of a group refers to monovalent hydrocarbon radicals having a straight or branched hydrocarbon chains having one or more triple bonds and containing from 2 to about 10 carbon atoms, more preferably from 2 to about 5 carbon atoms. Examples of alkynyl radicals include ethynyl, propynyl, (propargyl), butyny,l and the like.

The term “aryl” as used herein, alone or as part of a group, includes an organic radical derived from an aromatic hydrocarbon by removal of one hydrogen, and includes monocyclic and polycyclic radicals, such as phenyl, biphenyl, naphthyl.

The term “alkoxy” as used herein, alone or as part of a group, refers to an alkyl ether radical wherein the term alkyl is as defined above. Examples of alkyl ether radical include methoxy, ethoxy, n-propoxy, isopropoxy, n-butoxy, isobutoxy, sec-butoxy, tert-butoxy, and the like.

The term “cycloalkyl” as used herein, alone or in combination, means a saturated or partially saturated monocyclic, bicyclic or tricyclic alkyl radical wherein each cyclic moiety contains from about 3 to about 8 carbon atoms, more preferably from about 3 to about 6 carbon atoms. Examples of such cycloalkyl radicals include cyclopropyl, cyclobutyl, cyclopentyl, cyclohexyl, and the like.

The term “cycloalkylalkyl” as used herein, alone or in combination, means an alkyl radical as defined above which is substituted by a cycloalkyl radical as defined above. Examples of such cycloalkylalkyl radicals include cyclopropylmethyl, cyclobutyl-methyl, cyclopentylmethyl, cyclohexylmethyl, 1-cyclopentylethyl, 1-cyclohexylethyl, 2-cyclopentylethyl, 2-cyclohexylethyl, cyclobutylpropyl, cyclopentylpropyl, cyclohexylbutyl, and the like.

The term “substituted” as used herein means that one or more of the hydrogen atoms bonded to carbon atoms in the chain or ring have been replaced with other substituents. Suitable substituents include monovalent hydrocarbon groups including alkyl groups such as methyl groups and monovalent heterogeneous groups including alkoxy groups such as methoxy groups.

The term “unsubstituted” as used herein means that the carbon chain or ring contains no other substituents other than carbon and hydrogen.

The term “branched” as used herein means that the carbon chain is not simply a linear chain. “Unbranched” means that the carbon chain is a linear carbon chain.

The term “saturated” as used herein means that the carbon chain or ring does not contain any double or triple bonds. “Unsaturated” means that the carbon chain or ring contains at least one double bond. An unsaturated carbon chain or ring may include more than one double bond.

The term “hydrocarbon group” means a chain of 1 to 25 carbon atoms, suitably 1 to 12 carbon atoms, more suitably 1 to 10 carbon atoms, and most suitably 1 to 8 carbon atoms. Hydrocarbon groups may have a linear or branched chain structure. Suitably the hydrocarbon groups have one branch.

The term “carbocyclic group” means a saturated or unsaturated hydrocarbon ring. Carbocyclic groups are not aromatic. Carbocyclic groups are monocyclic or polycyclic. Polycyclic carbocyclic groups can be fused, spiro, or bridged ring systems. Monocyclic carbocyclic groups contain 4 to 10 carbon atoms, suitably 4 to 7 carbon atoms, and more suitably 5 to 6 carbon atoms in the ring. Bicyclic carbocyclic groups contain 8 to 12 carbon atoms, preferably 9 to 10 carbon atoms in the rings.

The term “heteroatom” means an atom other than carbon e.g., in the ring of a heterocyclic group or the chain of a heterogeneous group. Preferably, heteroatoms are selected from the group consisting of sulfur, phosphorous, nitrogen and oxygen atoms. Groups containing more than one heteroatom may contain different heteroatoms.

The term “heterocyclic group” means a saturated or unsaturated ring structure containing carbon atoms and 1 or more heteroatoms in the ring. Heterocyclic groups are not aromatic. Heterocyclic groups are monocyclic or polycyclic. Polycyclic heteroaromatic groups can be fused, spiro, or bridged ring systems. Monocyclic heterocyclic groups contain 4 to 10 member atoms (i.e., including both carbon atoms and at least 1 heteroatom), suitably 4 to 7, and more suitably 5 to 6 in the ring. Bicyclic heterocyclic groups contain 8 to 18 member atoms, suitably 9 or 10 in the rings.

The term “imine” or “imino”, as used herein, unless otherwise indicated, includes a functional group or chemical compound containing a carbon-nitrogen double bond. The expression “imino compound”, as used herein, unless otherwise indicated, refers to a compound that includes an “imine” or an “imino” group as defined herein.

The term “hydroxyl”, as used herein, unless otherwise indicated, includes —OH.

The terms “halogen” and “halo”, as used herein, unless otherwise indicated, include a chlorine, chloro, Cl; fluorine, fluoro, F; bromine, bromo, Br; or iodine, iodo, or I.

The term “cyano”, as used herein, unless otherwise indicated, includes a —CN group.

The term “alcohol”, as used herein, unless otherwise indicated, includes a compound in which the hydroxyl functional group (—OH) is bound to a carbon atom. In particular, this carbon center should be saturated, having single bonds to three other atoms.

The term “solvate” is intended to mean a solvate form of a specified compound that retains the effectiveness of such compound. Examples of solvates include compounds of the invention in combination with, for example: water, isopropanol, ethanol, methanol, dimethylsulfoxide (DMSO), ethyl acetate, acetic acid, or ethanolamine.

The term “mmol”, as used herein, is intended to mean millimole. The term “equiv”, as used herein, is intended to mean equivalent. The term “mL”, as used herein, is intended to mean milliliter. The term “g”, as used herein, is intended to mean gram. The term “kg”, as used herein, is intended to mean kilogram. The term “μg”, as used herein, is intended to mean micrograms. The term “h”, as used herein, is intended to mean hour. The term “min”, as used herein, is intended to mean minute. The term “M”, as used herein, is intended to mean molar. The term “μL”, as used herein, is intended to mean microliter. The term “μM”, as used herein, is intended to mean micromolar. The term “nM”, as used herein, is intended to mean nanomolar. The term “N”, as used herein, is intended to mean normal. The term “amu”, as used herein, is intended to mean atomic mass unit. The term “° C.”, as used herein, is intended to mean degree Celsius. The term “wt/wt”, as used herein, is intended to mean weight/weight. The term “v/v”, as used herein, is intended to mean volume/volume. The term “MS”, as used herein, is intended to mean mass spectroscopy. The term “HPLC”, as used herein, is intended to mean high performance liquid chromatograph. The term “RT”, as used herein, is intended to mean room temperature. The term “e.g.”, as used herein, is intended to mean example. The term “N/A”, as used herein, is intended to mean not tested.

As used herein, the expression “pharmaceutically acceptable salt” refers to pharmaceutically acceptable organic or inorganic salts of a compound of the invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, or pamoate (i.e., 1,1′-methylene-bis-(2-hydroxy-3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counterions. Hence, a pharmaceutically acceptable salt can have one or more charged atoms and/or one or more counterion. As used herein, the expression “pharmaceutically acceptable solvate” refers to an association of one or more solvent molecules and a compound of the invention. Examples of solvents that form pharmaceutically acceptable solvates include, but are not limited to, water, isopropanol, ethanol, methanol, DMSO, ethyl acetate, acetic acid, and ethanolamine. As used herein, the expression “pharmaceutically acceptable hydrate” refers to a compound of the invention, or a salt thereof, that further includes a stoichiometric or non-stoichiometric amount of water bound by non-covalent intermolecular forces.

The terms “Isomer,” “isomeric form,” “stereochemically isomeric forms,” or “stereolsomeric forms,” as used herein, defines all possible isomeric as well as conformational forms, made up of the same atoms bonded by the same sequence of bonds but having different three-dimensional structures which are not interchangeable, which compounds or intermediates obtained during said process may possess. Unless otherwise mentioned or indicated, the chemical designation of a compound encompasses the mixture of all possible stereochemically isomeric forms which said compound may possess. Said mixture may contain all diastereoisomers, epimers, enantiomers, and/or conformers of the basic molecular structure of said compound. More in particular, stereogenic centers may have the R- or S-configuration, diastereoisomers may have a syn- or anti-configuration, substituents on bivalent cyclic saturated radicals may have either the cis- or trans-configuration and alkenyl radicals may have the E or Z-configuration. All stereochemically isomeric forms of said compound both in pure form or in admixture with each other are intended to be embraced within the scope of the present invention.

Specific embodiments disclosed herein may be further limited in the claims using “consisting of” or “consisting essentially of” language, rather than “comprising”. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

As various changes could be made in the above-described materials and methods without departing from the scope of the invention, it is intended that all matter contained in the above description and in the examples given below, shall be interpreted as illustrative and not in a limiting sense.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Dimethyl Itaconate Down-Regulates IKBζ Induction

Dimethyl itaconate (DI) is a cell permeable chemical analog of itaconate, naturally produced metabolite in inflammatory macrophages. The inventors have shown that DI treatment of macrophages selectively down-regulates LPS-induced production of inflammatory cytokines such as IL1b, IL6, IL12, but not TNF. The present work deciphered the molecular mechanism responsible for selective action of DI on gene transcription. The transcriptional profile of DI treated cells showed a signature typical for oxidative and xenobiotic/electrophilic stress. In line with an oxidative signature, DI-treated cells exhibited increased intracellular ROS production and substantial drop in cellular glutathione levels, events typical for Nrf2-inducing agents. A number of Nrf2-triggering agents modulate inflammatory conditions by down-regulating nuclear factor-k B (NFkB) pathway, however DI did not show effect on classical NFkB signaling as shown by activation of key NFkB regulators and p65 nuclear localization in LPS-activated cells. Therefore its selective action towards NFkB primary response genes was tested. This example shows that DI treatment selectively down-regulates Ikb-ζ induction in an Nrf2-independent manner.

Similarly, DI showed to be potent inhibitor of Ikb-ζ in IL-17A-stimulated keratinocytes where Ikb-ζ plays a key role in induction of psoriasis-associated genes. Moreover, DI was capable to ameliorate psoriatic pathology associated in murine model of psoriasis. Similarly, DI was able to ameliorate clinical development of EAE disease. Finally, since Ikb-ζ has been also shown to control survival of ABC but not GCB DLBCL cell lines, DI potential to limit proliferation of DLBCL cell lines was tested. DI was selectively toxic to ABC but not GCB subtypes of DLBCL cell lines. Therefore, DI can be used as a therapeutic option for treatment of Ikbz/II17 associated diseases such as 1) autoinflammatory diseases (e.g., psoriasis and multiple sclerosis) or 2) Ikbz-dependent tumors such as ABC subtype of DLBCL.

Antioxidant Signature of DI Treated Macrophages

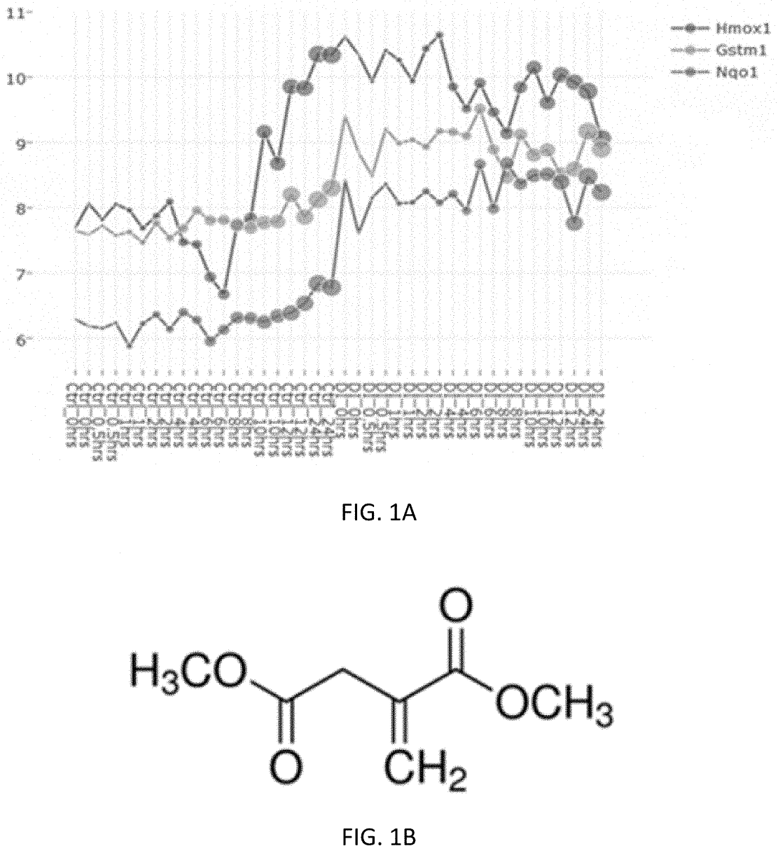

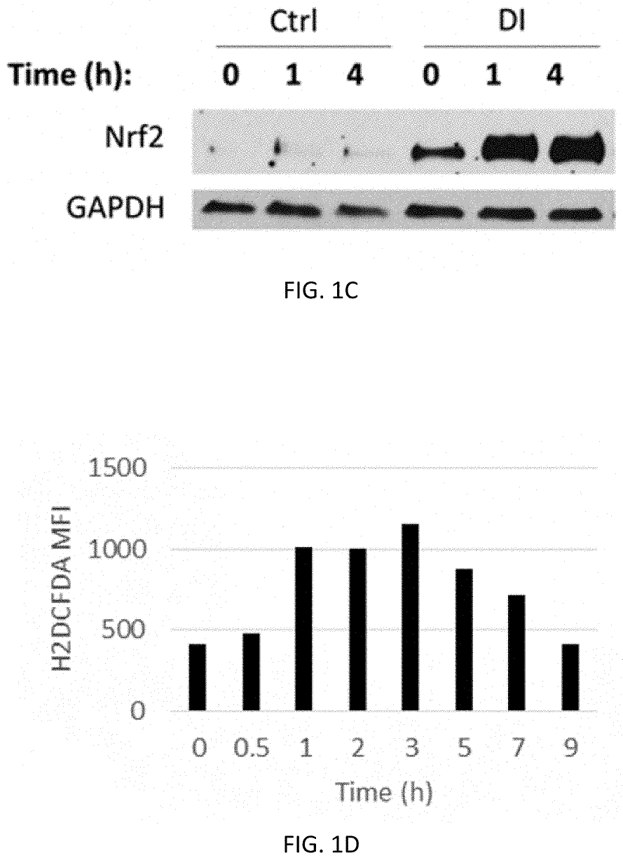

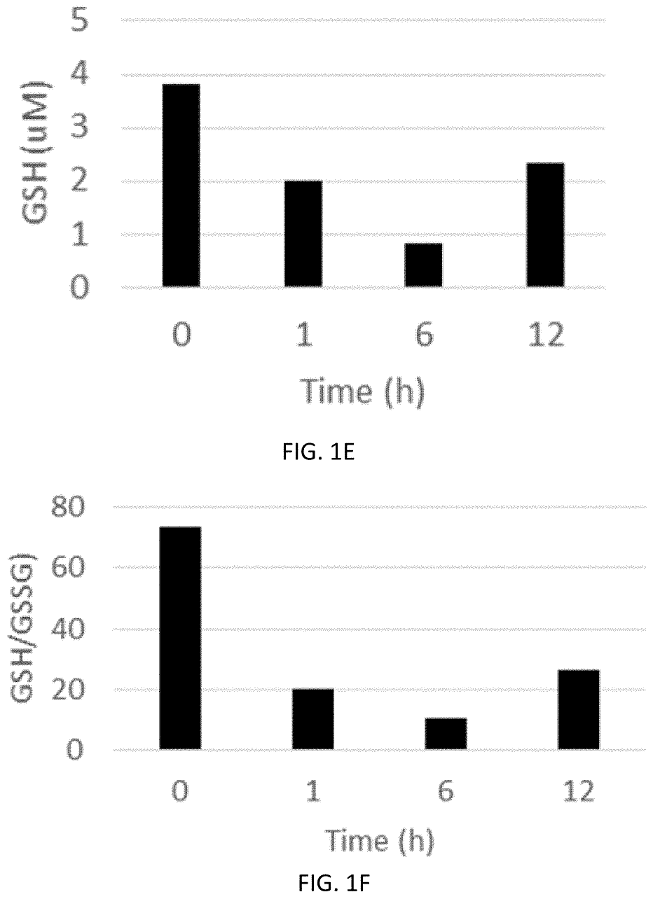

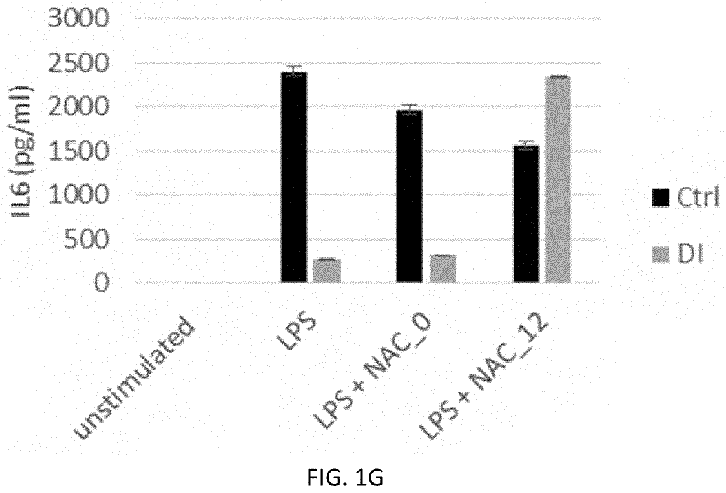

To elucidate the mechanism of DI action in macrophage effector functions this study explored DI-up-regulated genes in mouse macrophages by global transcriptomic profiling. Analysis of DI-pretreated cells identified genes significantly enriched by DI treatment as several prototypical oxidative stress response genes that are commonly up-regulated in cells exposed to oxidative and xenobiotic/electrophilic stress (see e.g., FIG. 1A). DI is a methyl ester of itaconic acid, or methylenesuccinic acid that poses electrophilic unsaturated bond which can undergo Michael additions with nucleophiles as SH groups in cysteine (see e.g., FIG. 1B). Nfr2/Keap1 antioxidant system is a master regulator of cellular response to electrophilic stress. A cysteine-rich protein Keap1 acts as a redox sensor and upon addition/oxidation of its sulfhydryl groups activates Nrf2 transcription factor. 12 h pretreatment of macrophages with DI triggered strong Nrf2 response as detected by Nrf2 western blot and induction of Nrf2 target genes (see e.g., FIG. 1C). These data suggest active intracellular presence of DI. In line with upregulation of oxidative signature pathways DI-treated cells exhibited increased intracellular ROS (see e.g., FIG. 1D) production and substantial drop in cellular GSH (see e.g., FIG. 1E) and GSH/GSSG ratio (see e.g., FIG. 1F).

It was shown that DI pretreatment of BMDMs down-regulates production of IL6, IL12, and IL1b, but not TNFα. To test whether neutralization of oxidative stress can diminish DI effect on IL6 production, N-acetylcysteine (NAC) was used in cells pretreated with DI and stimulated with LPS. NAC addition completely abrogated effect of DI when added simultaneously with DI but not when added at the time of LPS stimulation (see e.g., FIG. 1G). These data suggest that DI-mediated cellular response build during 12 h of pretreatment leads to changes in cellular state that results in down-regulation of subset of inflammatory genes in macrophages.

DI Down-Regulates Ikb-ζ Induction

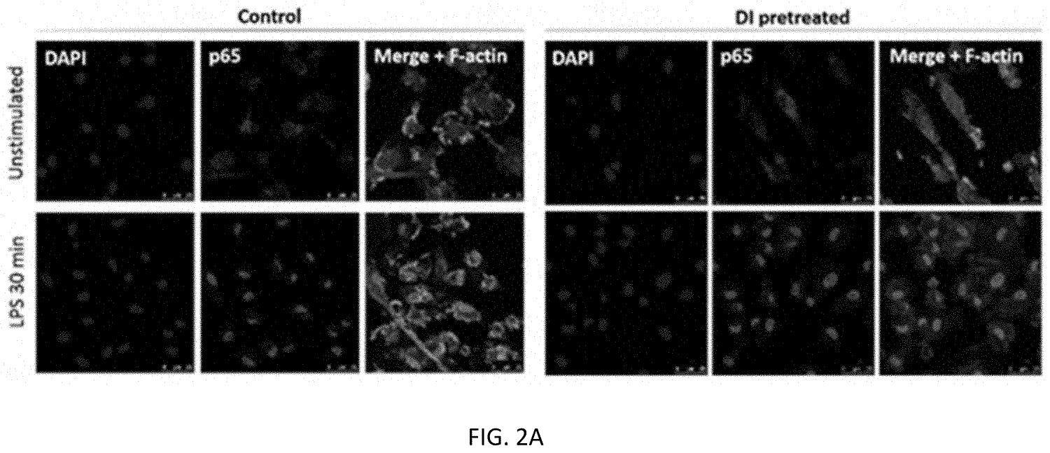

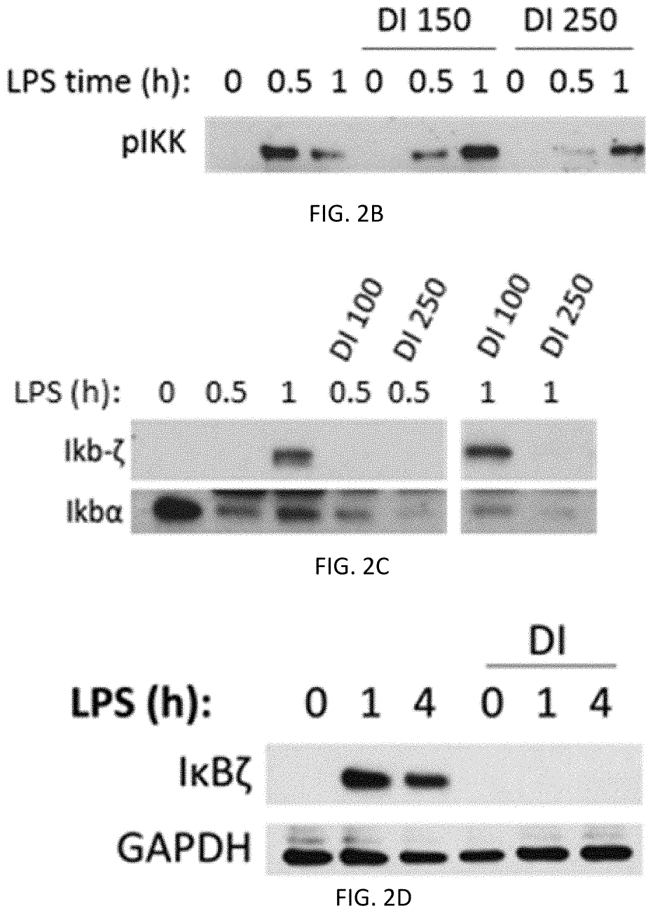

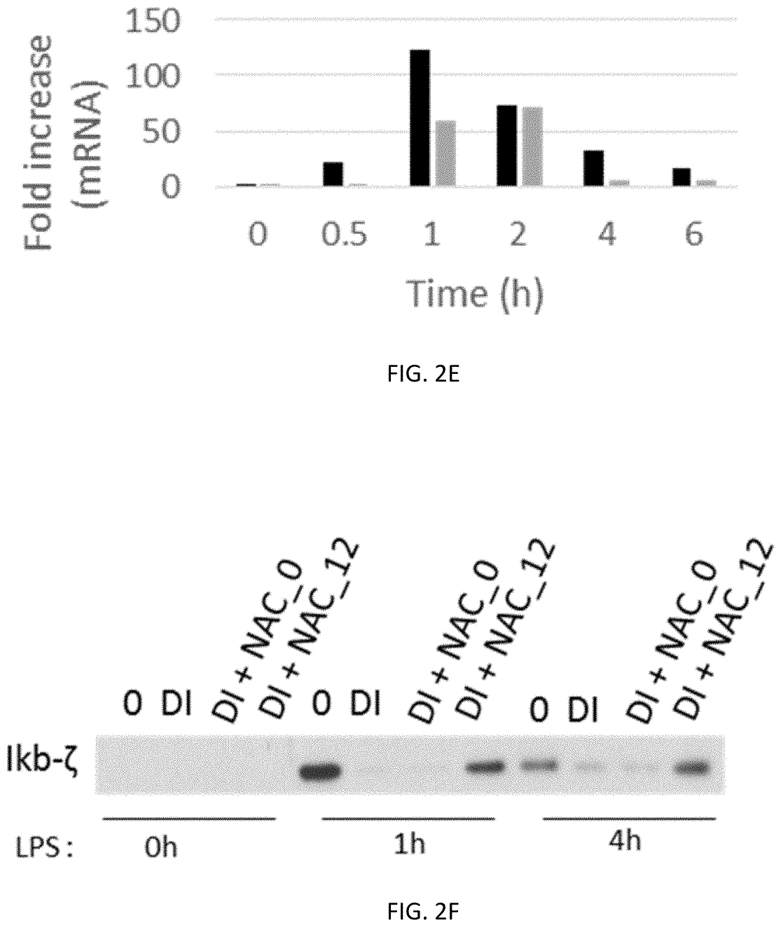

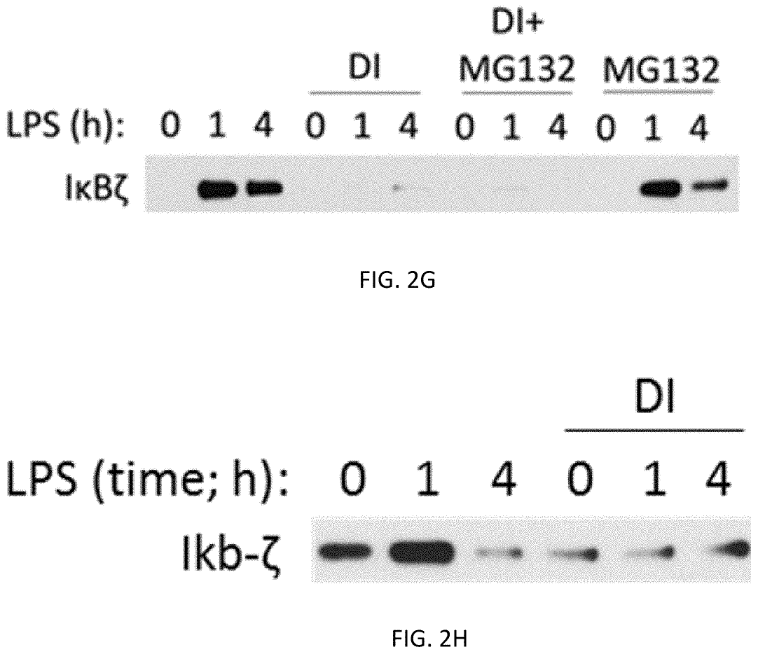

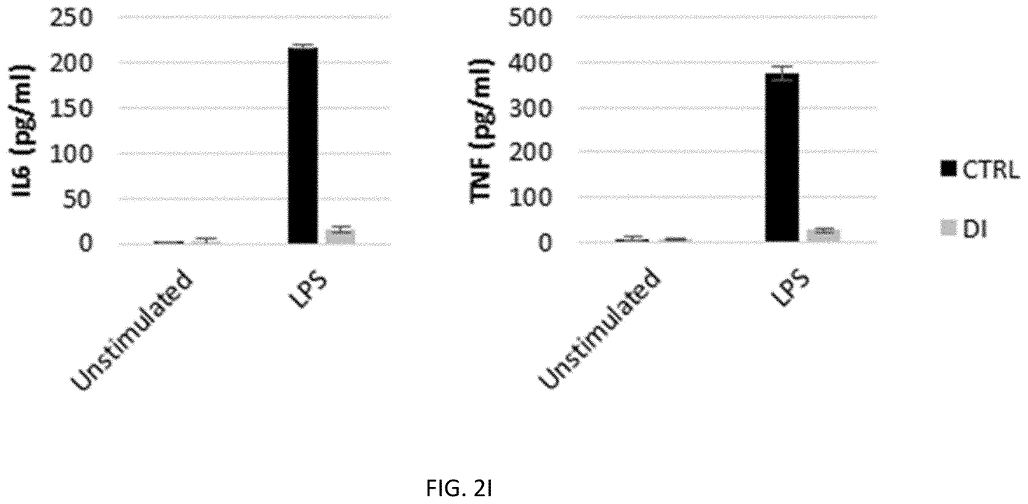

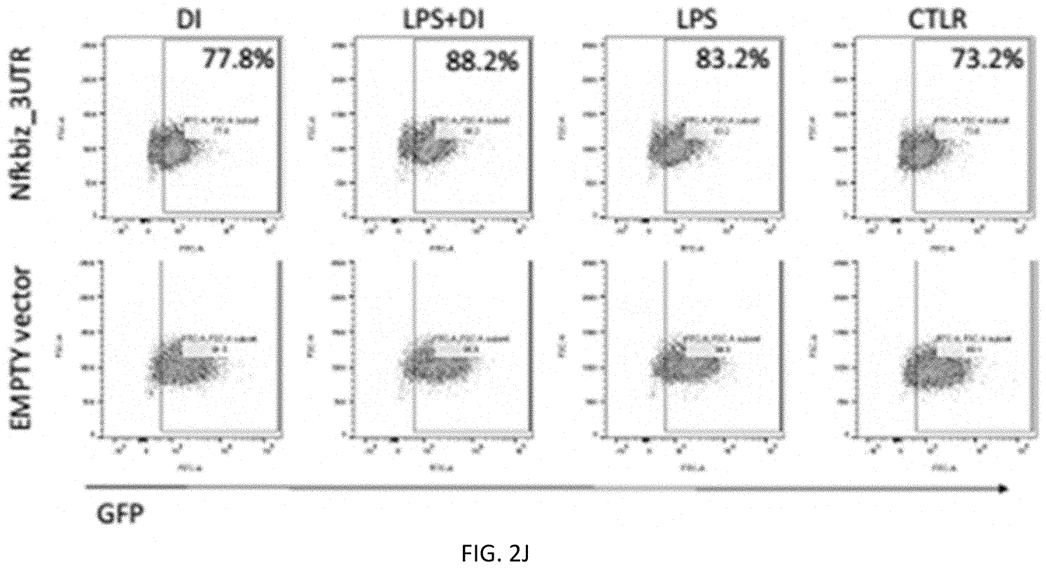

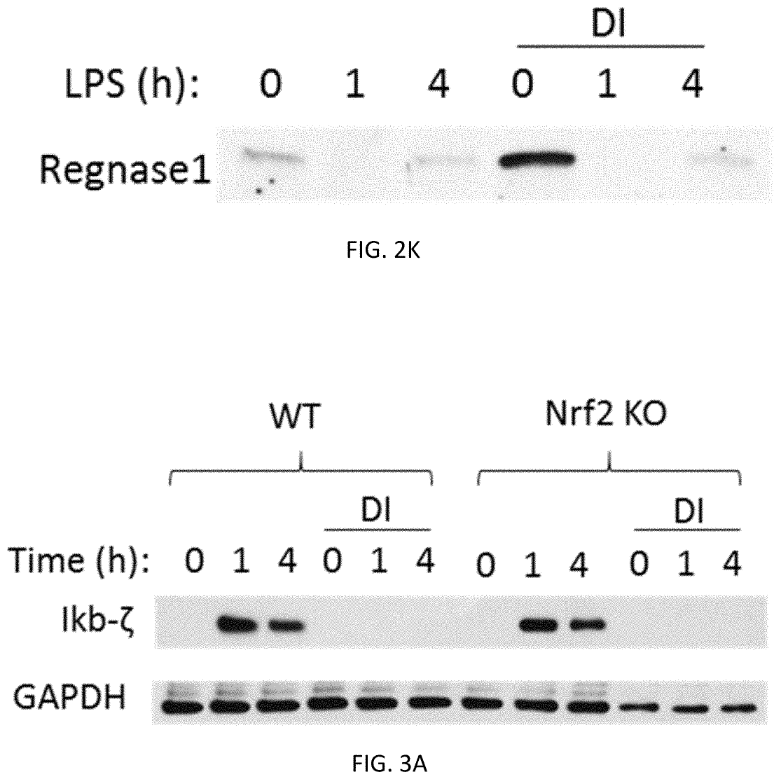

Compounds with related chemical properties as DI (e.g., DMF, DEM) have been shown to down-regulate NFkB response in immune cells. Since canonical NFkB response was not affected by DI in concentrations effective to inhibit IL6 production but not TNF as detected by p65 nuclear translocation (see e.g., FIG. 2A), IKK activation (see e.g., FIG. 2B), and IkBa degradation (see e.g., FIG. 2C), possible NFkB target genes with selective function in cytokine response was explored. Ikb-ζ is an inducible nuclear protein that regulates Toll/IL-1-receptor-mediated gene expression and has been shown to be critical for production of IL6 but not TNFα in macrophages. LPS induced strong expression of Ikb-ζ as detected on mRNA and protein level and DI pretreatment resulted in almost complete inhibition of Ikb-ζ induction of protein level (see e.g., FIG. 2D) with only moderate effect on mRNA level (see e.g., FIG. 2E). In agreement with neutralizing effect of NAC on downregulation of cytokine production by DI, NAC pretreatment also restored Ikb-ζ production in DI-pretreated cells (see e.g., FIG. 2F). Diminished Ikb-ζ protein levels were not due to proteasomal degradation since inhibition of proteasome with MG132 did not affect Ikb-ζ levels in DI treated cells (see e.g., FIG. 2G). These data suggest that DI acts on NFkBiz mRNA expression and/or interferes with posttranscriptional regulation of Ikb-ζ. Ikb-ζ expression in LPS-stimulated human monocytes was also tested. DI treatment of monocytes resulted in decreased Ikb-ζ protein levels (see e.g., FIG. 2H) and inhibition of cytokine production (see e.g., FIG. 2I).

Ikb-ζ mRNA has been previously shown to be regulated by several mechanisms affecting mRNA 3-UTR elements. Therefore Ikb-ζ 3-UTR reporter system was utilized in mouse BV2 immortalized cell line. DI pretreatment and LPS stimulation did not affect expression of reporter gene GFP as detected by flow cytometry (see e.g., FIG. 2J). Ikb-ζ mRNA has been shown to be negatively regulated by Regnase-1. Upon various cell stimuli (T cells, macrophages), this regulation is relieved by Regnase-1 degradation by MALT-1. DI treatment up-regulated Regnase-1 in unstimulated macrophages that was subsequently degraded upon LPS stimulation to a similar extent as in control cells (see e.g., FIG. 2K).

DI Acts on Ikb-ζ Induction in NRF2-Independent Manner

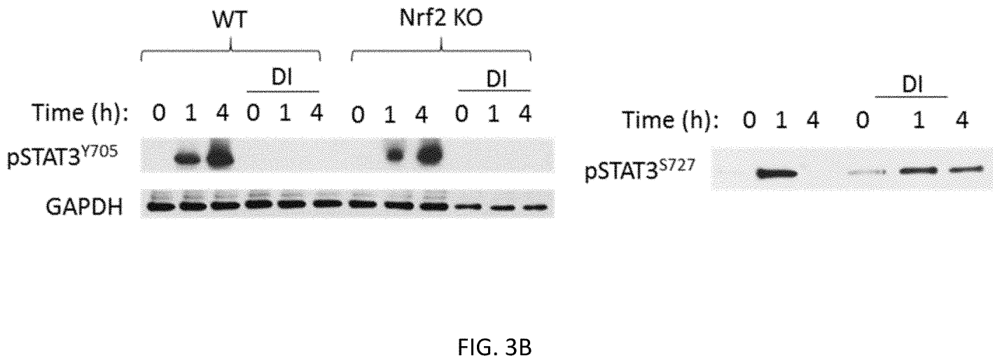

To investigate whether effect of DI on Ikb-ζ occurs through activation of Nrf2 pathway Ikb-ζ induction was analyzed in DI-pretreated Nrf2-deficient cells. As shown herein, (see e.g., FIG. 3A) lack of Nrf2 did not affect inhibition of Ikb-ζ induction proving that DI-effect on Ikb-ζ occurs in Nrf2-independent manner.

DI Acts on STAT3 Activation in NRF2-Independent Manner

Inhibition of STAT3 in macrophages leads to down-regulation of IL6 but not TNFα production. It was tested whether beside specific NFkB components DI could have additional effect of STAT3 activation. STAT3 was activated upon 1 h and 4 h of LPS stimulation as detected by Y705 site phosphorylation (see e.g., FIG. 3B). Nrf2 KO showed no differences in extent of inhibition of STAT3 activation compared with WT cells. Together these data show that DI inhibits STAT3 activation in macrophages by mechanism that does not involve Nrf2. DI also affecting S727 phosphorylation in WT cells, which manifested as increased phosphorylation in unstimulated cells and decreased induction in phosphorylation after LSP stimulation compared to DI-untreated cells.

DI down-regulates IL17-induced Ikb-ζ and STAT3 activation in keratinocytes

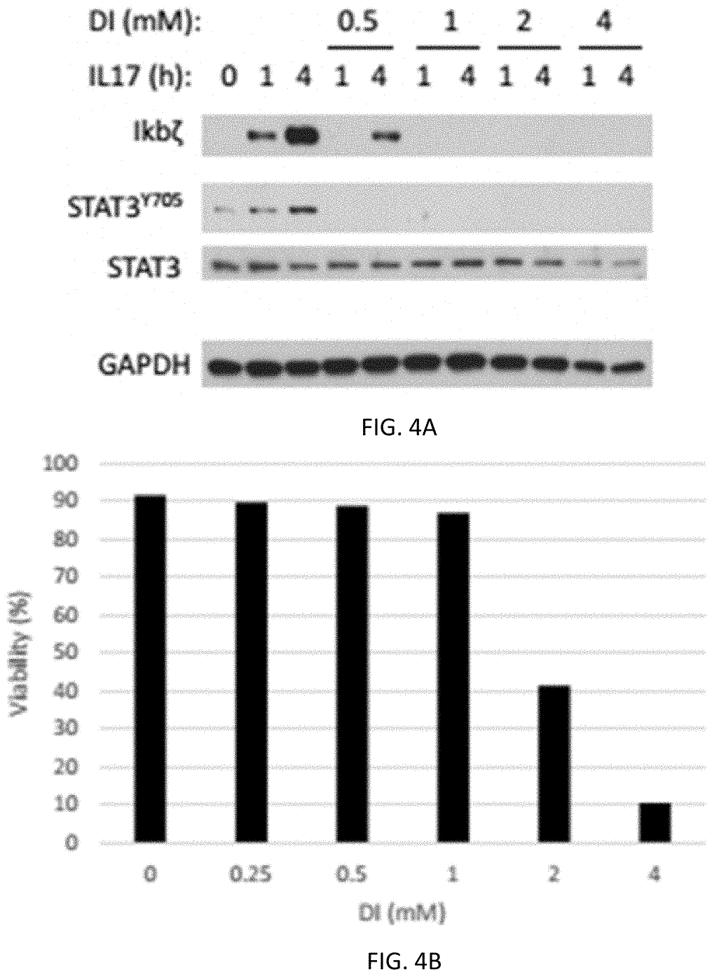

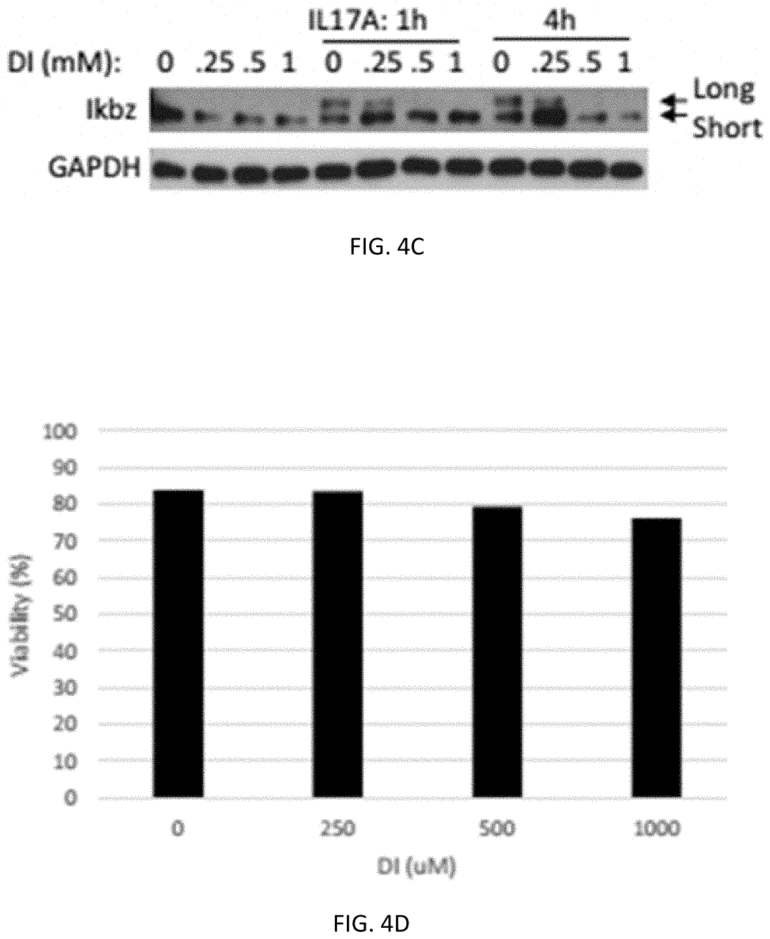

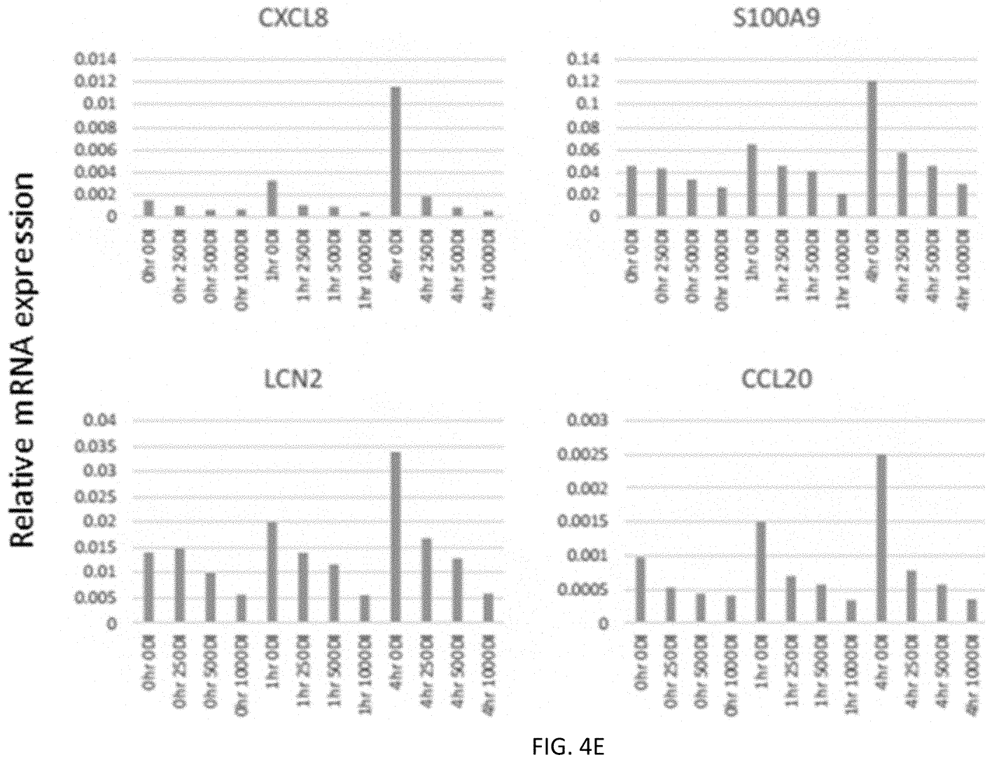

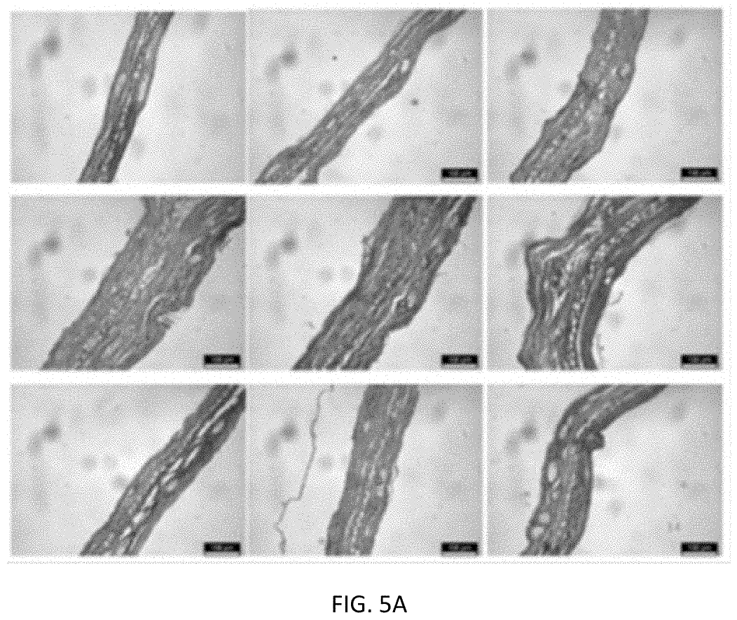

Ikb-ζ has been shown to be induced by stimulation of IL17 receptor in different cellular systems. It was hypothesized that DI could be potentially used to regulate Ikb-ζ induction in keratinocytes. To test this hypothesis, human immortalized HaCat kereatinocyte cell line was used, and DI effect on IL17A-mediated Ikb-ζ induction was tested. Ikb-ζ induction increased during 4 h of IL17A stimulation and 12 h of DI pretreatment at non-toxic concentrations almost completely inhibited this induction (see e.g., FIG. 4A). All tested DI concentrations were also effective to inhibit STAT3 activation. To ensure this was not due to a loss of cell viability, the cell viability was determined by flow cytometry (see e.g., FIG. 4B). To confirm these data in primary cells human primary keratinocytes were generated. In primary cells only long form of Ikb-ζ has been induced by IL17. Presence of short form of Ikb-ζ might reflect a donor specific condition. Induction of the long form of Ikb-ζ was inhibited by DI treatment (see e.g., FIG. 4C). Viability of primary keratinocytes was not affected by DI treatment (see e.g., FIG. 4D). In line with Ikb-ζ down-regulation, expression of IL-17A signature genes including Lcn2 and S100a9 was also inhibited as detected by qPCR (see e.g., FIG. 4E).

DI Ameliorates Psoriatic Pathology in Mouse Model

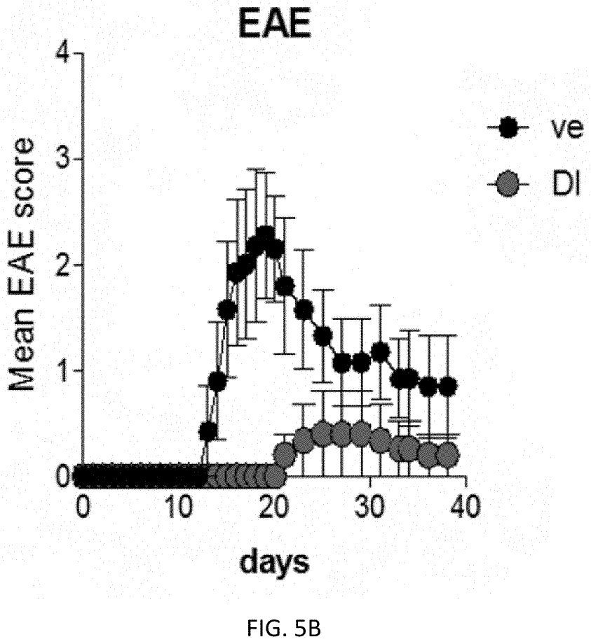

NFKBIZ was identified as a new psoriasis susceptibility locus and Ikb-ζ has been shown to act as a direct transcriptional activator of TNFα/IL17-inducible psoriasis-associated genes and as a key driver in development of psoriasis. To characterize effect of DI in the pathogenesis of psoriasis a mouse model of psoriasis-like skin inflammation (induced by topical application of the Toll-like receptor (TLR) agonist imiquimod) and which in many ways models human psoriasis was used. DI was administered to mice daily, which led to significant improvement of the psoriatic pathology (see e.g., FIG. 5A).

DI ameliorates pathology in the EAE model of multiple sclerosis

Similarly, the same daily regimen of DI administration was used in vivo in the EAE model of the mice and found that disease was ameliorated (see e.g., FIG. 5B)

DI Selectively Inhibits Growth of ABC Subtype of DLBCL Lymphoma

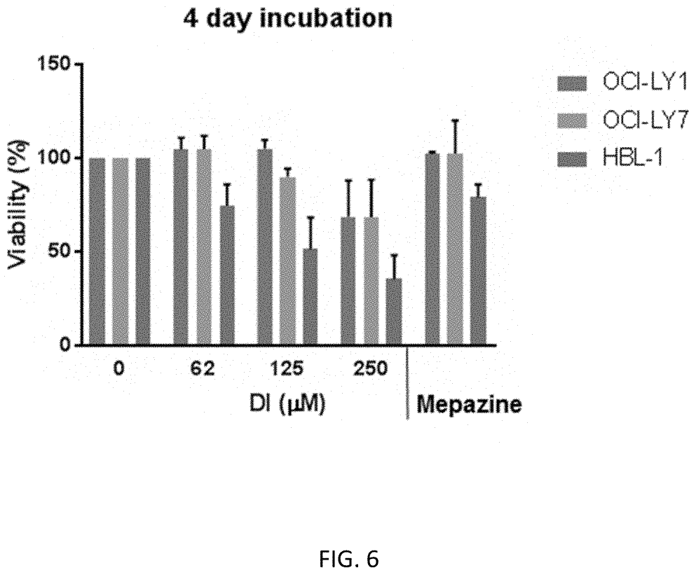

Constitutive activation of the NFkB pathway is a hallmark of the activated B-cell-like (ABC) subtype of diffuse large B-cell lymphoma (DLBCL). Ikb-ζ has been shown to be up-regulated in ABC compared to germinal center B-cell-like (GCB) DLBCL primary patient samples and silencing of Ikb-ζ by RNA interference was toxic to ABC but not to GCB DLBCL cell lines. These findings suggest that Ikb-ζ might represent a promising molecular target for future therapies of ABC lymphoma subtypes. To test whether DI potential to down-regulate Ikb-ζ expression can be exploited in treatment of ABC DLBCL HBL1 cells line (ABC) and LY1 and LY7 (GCB) cell lines were treated with various concentrations of DI for 4 days. DI dosage was refreshed every day and mepazine, an inhibitor of MALT1 proteolytic activity was used as control compound that has proven selective toxicity to ABC growths. Mepazine treatment inhibited growth of HBL1 but not LY1 and LY7 (see e.g., FIG. 6). DI at dose 125-250 μM showed selective toxicity towards ABC but not GCB cells. These data suggest DI as a potent and selective inhibitor of ABC growth and therapeutic option in ABC subtypes of DLBCL lymphoma.

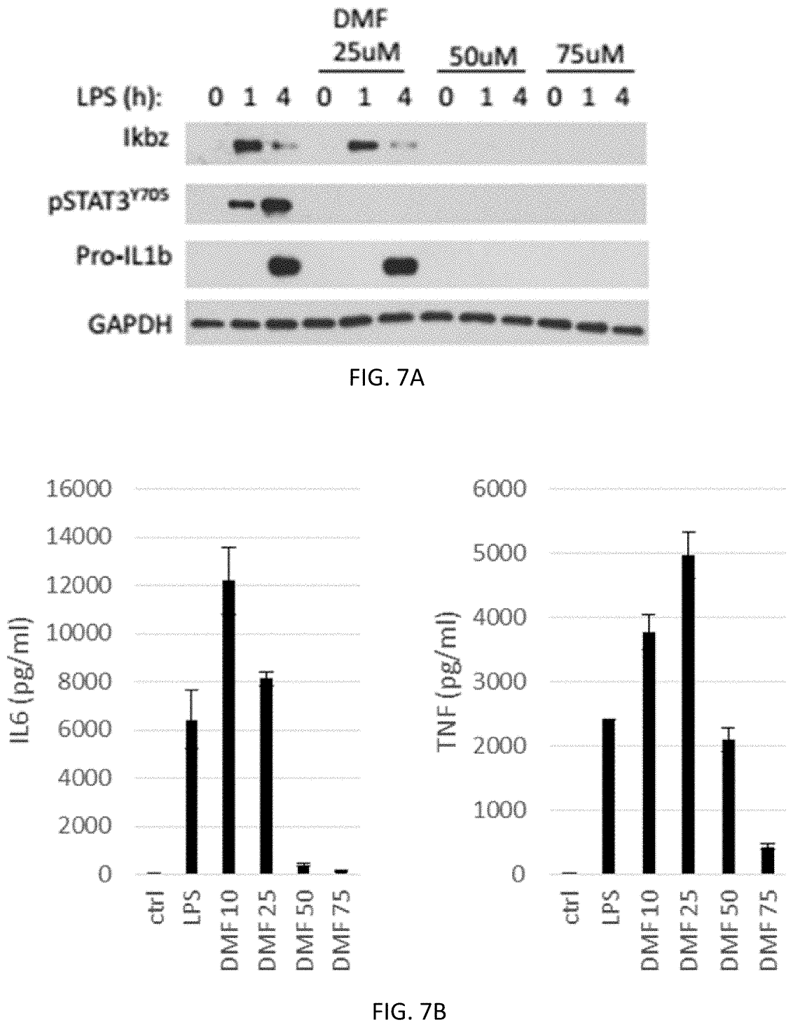

Dimethyl fumarate (DMF) is a currently approved drug for treatment of psoriasis. Structure of DMF resembles Michael acceptor chemical features of DI. DMF has been shown to modulate macrophage inflammatory cytokine production in Nrf2-independent manner. To compare DI and DMF effects in LPS-stimulation macrophages, BMDMs were pretreated with various concentrations of DMF and Ikb-ζ induction and STAT3 activation were analyzed. At concentration effective to inhibit IL6 but not TNF production, DMF strongly inhibited Ikb-ζ induction and all tested concentrations of DMF were effective to inhibit STAT3 activation. These data strongly support the notion that DMF might exert some of its physiological effects through inhibition of Ikb-ζ and STAT3 inhibition. Based on the obtained data it DMF can also be effective in treatment of ABC DLBCL lymphoma subtypes (see e.g., FIG. 7).

### Example 1 References

1. Lampropoulou, V. et al. Itaconate Links Inhibition of Succinate Dehydrogenase with Macrophage Metabolic Remodeling and Regulation of Inflammation. Cell Metab. 24, 158-166 (2016).
2. Schulze-Topphoff, U. et al. Dimethyl fumarate treatment induces adaptive and innate immune modulation independent of Nrf2. Proc Natl Acad Sci USA 113, 4777-4782 (2016).
3. McGuire, V. A. et al. Dimethyl fumarate blocks pro-inflammatory cytokine production via inhibition of TLR induced M1 and K63 ubiquitin chain formation. Sci. Rep. 6, 31159 (2016).
4. Yamamoto, M. et al. Regulation of Toll/IL-1-receptor-mediated gene expression by the inducible nuclear protein IκBζ. Nature 430, 218-222 (2004).
5. Nogai, H. et al. I k B-z controls the constitutive NF-k B target gene network and survival of ABC DLBCL. Blood 122, 2242-2251 (2015).
6. Nagel, D. et al. Pharmacologic inhibition of MALT1 protease by phenothiazines as a therapeutic approach for the treatment of aggressive ABC-DLBCL. Cancer Cell 22, 825-37 (2012).

### Example 2: Dimethyl Itaconate Down-Regulates Ikbζ-Mediated Inflammatory Responses

Dimethyl itaconate (DI) is a cell permeable chemical analog of itaconate, metabolite that is naturally produced by inflammatory macrophages. The inventors have shown that DI treatment of bone marrow-derived macrophages (BMDMs) selectively inhibits LPS-induced production of inflammatory cytokines such as IL-1β, IL-6, IL-12, but not TNF [1], In the present work the molecular mechanism responsible for selective action of DI on gene transcription was deciphered.

Transcriptional profile of DI treated cells shows signature typical for oxidative and xenobiotic/electrophilic stress. DI-treated cells exhibited increased intracellular ROS production and drop in cellular glutathione levels, events typical for Nrf2-inducing agents. A number of Nrf2-triggering agents modulate inflammatory conditions by down-regulating the NF-κB pathway [2], however, at concentrations effective to inhibit IL-6, DI did not show inhibition of NF-κB signaling. Therefore selective action of DI towards NF-κB primary response gene, Ikbζ, which selectively regulates IL-6, IL-12 but not TNF transcription, was tested. This example shows that DI treatment down-regulates LPS-mediated Ikbζ protein induction in macrophages. This effect can be reversed by N-acetylcysteine and is independent of Nrf2-mediated antioxidant response. Analysis of the genes up- or down-regulated by DI in Nrf2-independent manner identified signature pathways connected to protein synthesis suggesting that DI may regulate Ikbζ by translation-associated mechanisms. Next the DI effect in other Ikbζ-dependent systems was tested. Ikbζ has been shown to act as a direct transcriptional activator of TNF/IL-17-inducible psoriasis-associated genes such as Defb4, S100a7a, S100a9a, or Lcn2 and as a key driver in development of psoriasis [4], Thus, DI the effect of DI on Ikbζ-induction in keratinocytes was tested. It was shown that DI effectively inhibits Ikbζ in IL-17A stimulated keratinocytes and down-regulates expression of Ikbζ-target genes. Moreover, DI is capable to ameliorate psoriatic pathology associated with murine model of psoriasis and down-regulates major Ikbζ target genes in skin tissue. Based on these data it is suggested that DI can be a therapeutic option for treatment of Ikbz/IL17 associated diseases.

DI Treated Macrophages Show Signature of Antioxidant Response

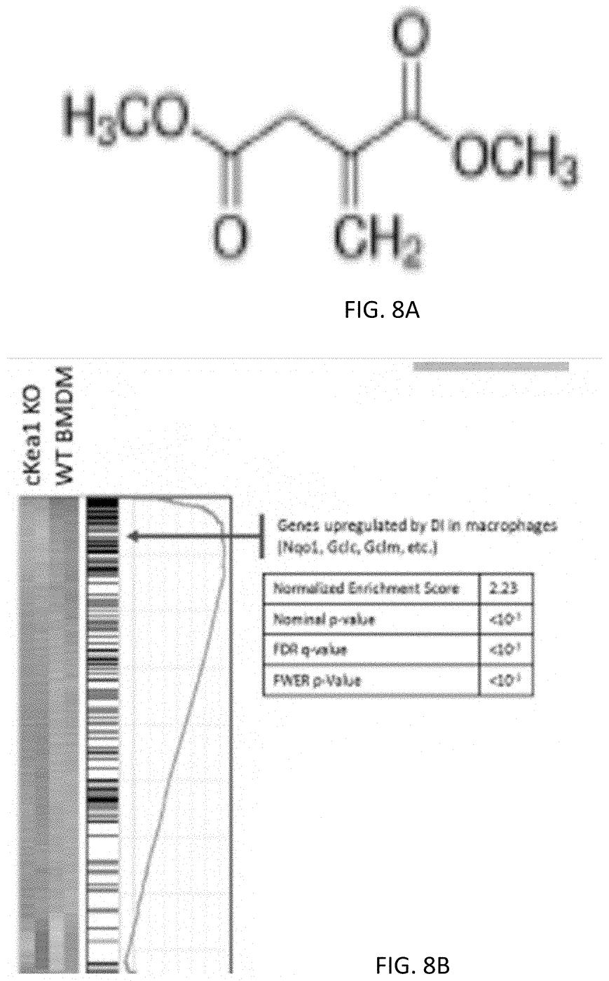

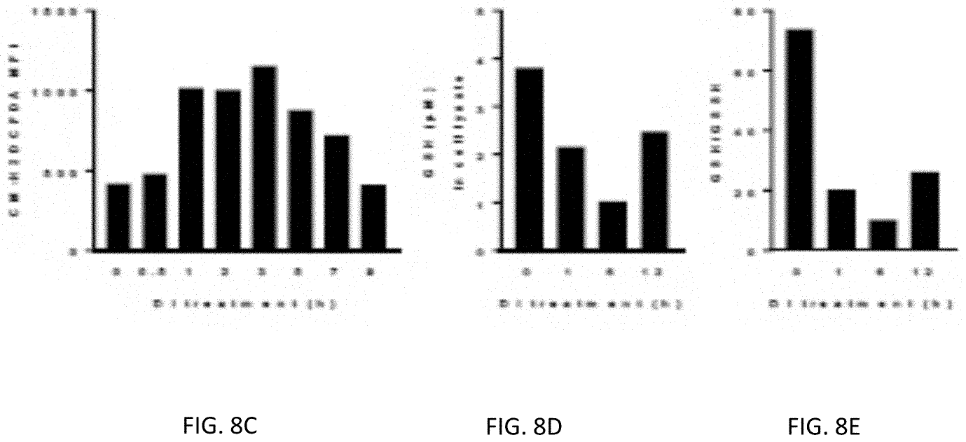

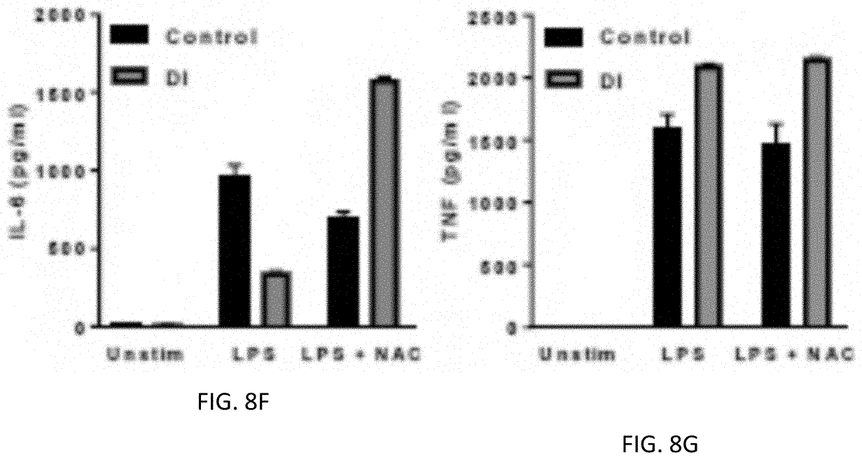

FIG. 8 shows (A) Structure of DI. (B) Comparison of transcriptional profiles of cKeap KO BMDMs and BMDMs treated with 250 μM DI for 12 h. (C) Triggering of Nrf2 expression and expression of Nrf2-targets (Nqo1, HO-1) in BMDMs treated as in B. (D) Analysis of intracellular DI uptake and identification of DI-glutathione adduct. (D) ROS production measured by detection of CM-H2DCFDA in BV2 cells treated with DI as in (B). (F) Glutathione depletion and GSH/GSSG ratio (G) in BMDMs treated with DI as in (B). (H) N-acetylcysteine (NAC) neutralizes effect of DI on cytokine production. BMDMs were treated with 250 μM DI and stimulated with LPS for 12 h. In some samples NAC was added simultaneously with DI.

DI does not Inhibit Signaling Through NF-κB Pathway

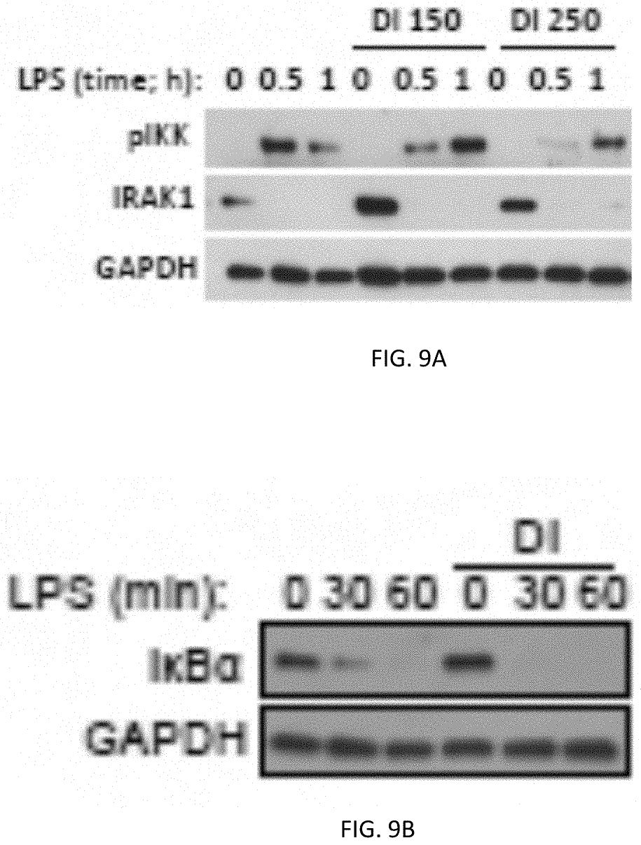

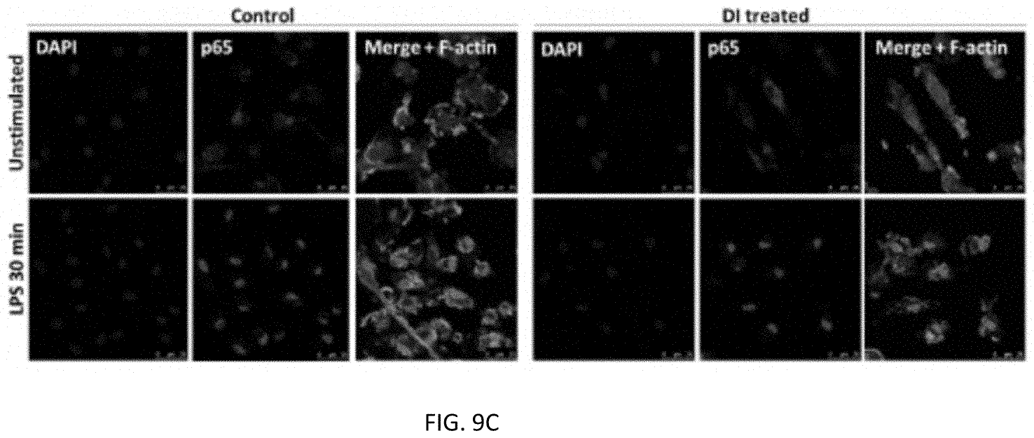

FIG. 9 (A) BMDMs were treated with 250 μM DI for 12 h and stimulated with LPS. Loss of IRAK1 detection upon LPS refers to its K63 ubiquitination [2] that correlates with activation. DI affected phosphorylation of IKK in time-dependent manner. (B) Degradation of Ikbζ is also not affected by DI treatment. (B) BMDMs were treated with DI as in A and fixed, permeabilized and stained for p65, F-Actin and nuclei. DI pretreatment does not inhibit nuclear translocation of p65 upon LPS stimulation.

DI Down-Regulates Ikbζ Induction

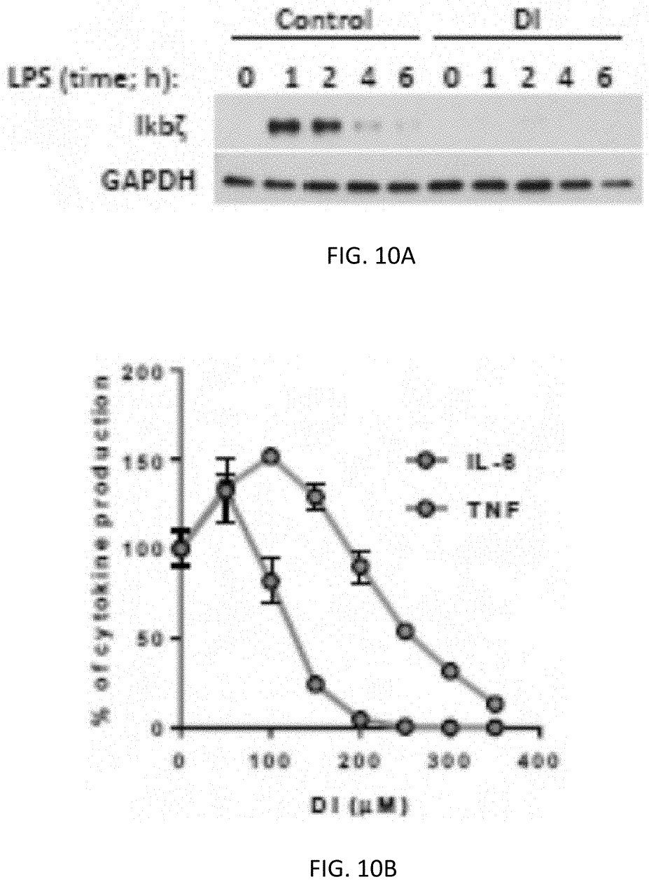

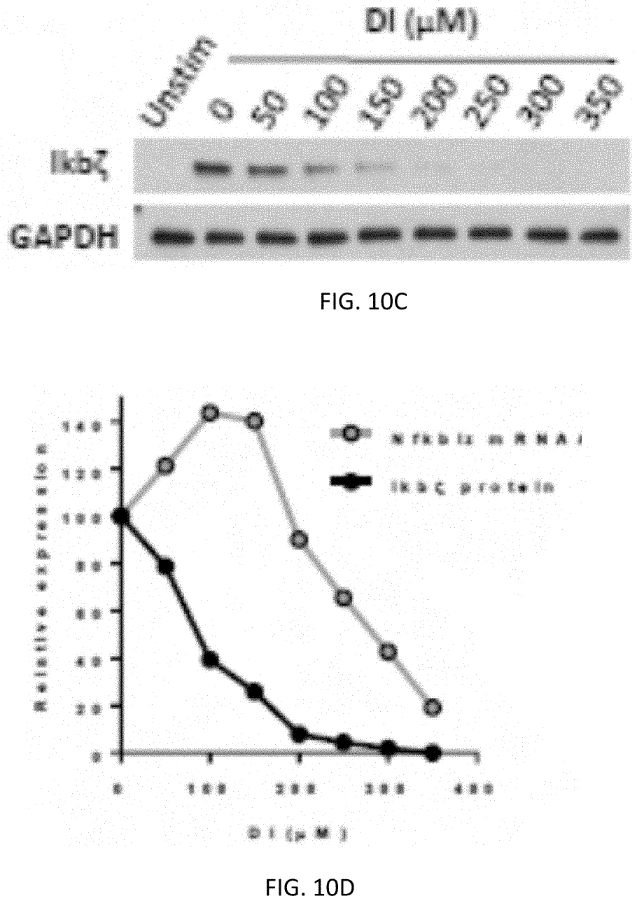

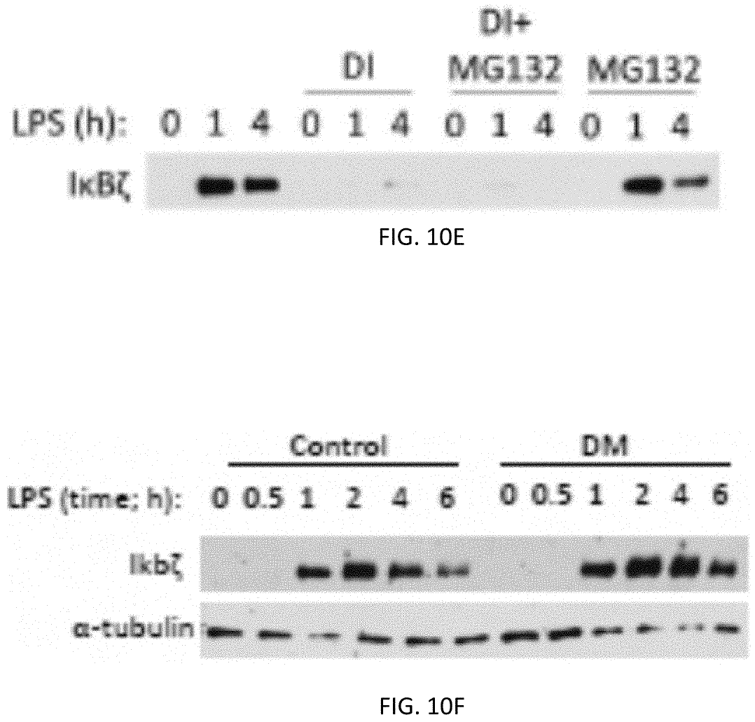

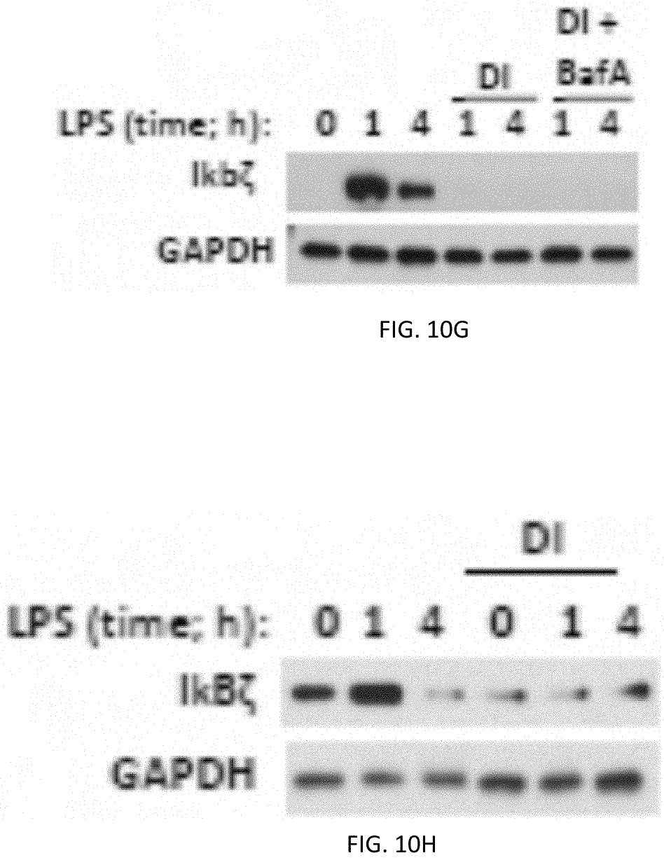

FIG. 10 (A) Detection of Ikbζ expression in BMDMs treated with DI for 12 h and stimulated with LPS. (B) Dose dependent effect of DI on cytokine production in BMDMs pretreated with DI for 12 h and stimulated with LPS for 4 h. (C) Dose dependent inhibition of Ikbζ expression in in cell treated as in B and stimulated with LPS for 1 h. (D) Nfkbiz mRNA expression in Ikbζ protein expression in cells treated as in (C). (D) Diminished Ikbζ protein expression is not due to the proteasomal degradation as shown by treatment of cells with MG132 or due to lysosomal/autophagy degradation as shown by treatment of cells with BafalomycinA (BafA) (E). (F) Dimethyl malonate (DM), a succinyl dehydrogenase inhibitor, does not affect Ikbζ expression. BMDMs were treated with 10 mM DM for 12 h and stimulated with LPS. (H) Detection of Ikbζ expression in human CD14+ monocytes treated with DI for 12 h and stimulated with LPS.

DI Effect on Ikbζ is Independent of Nrf2

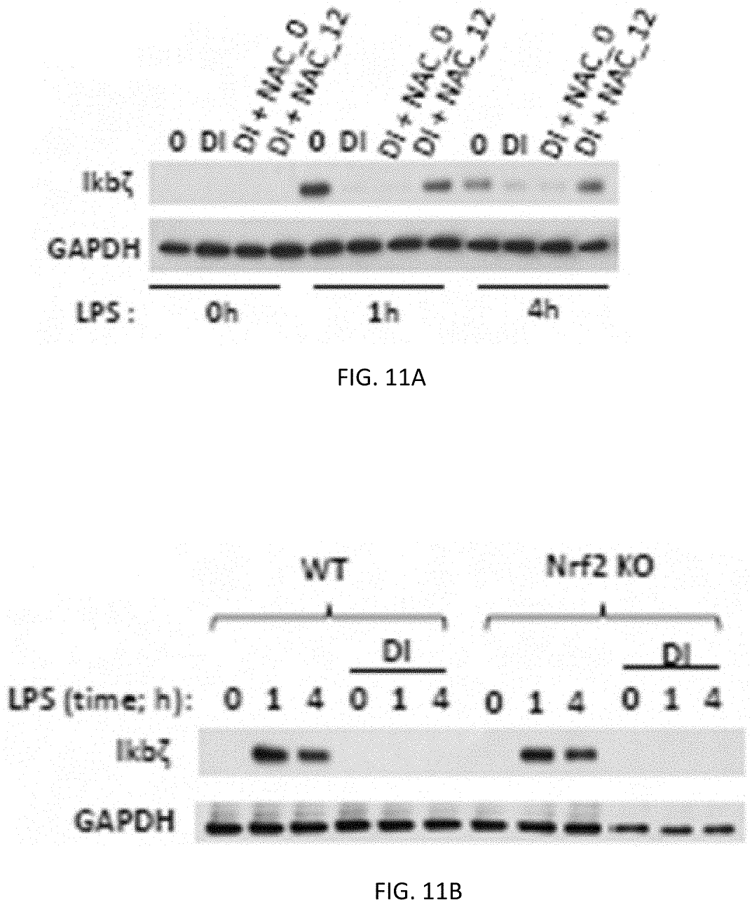

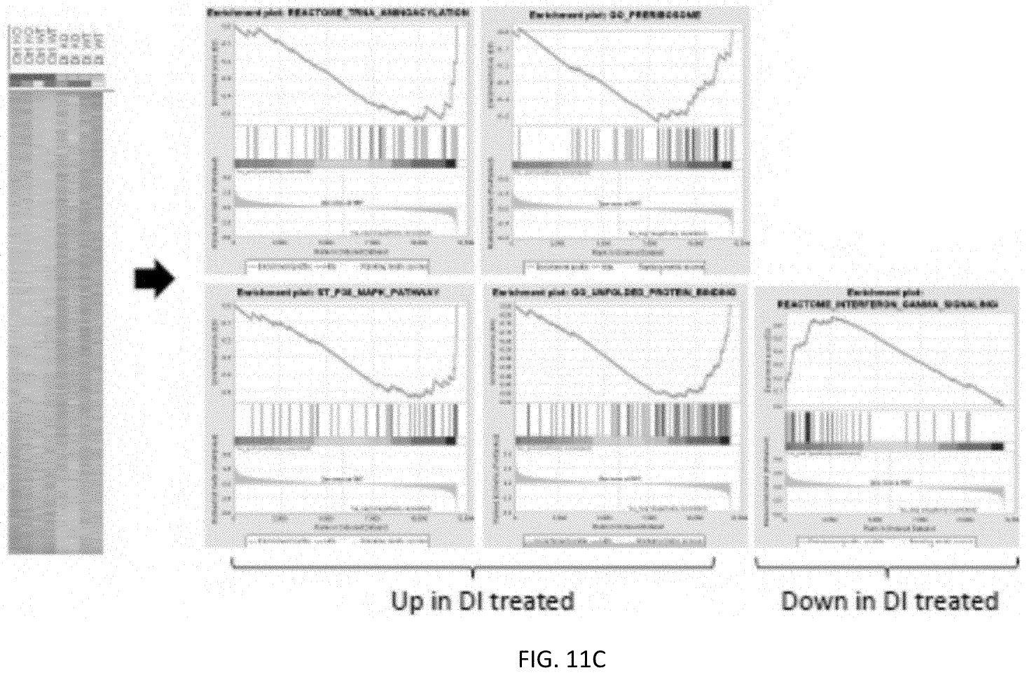

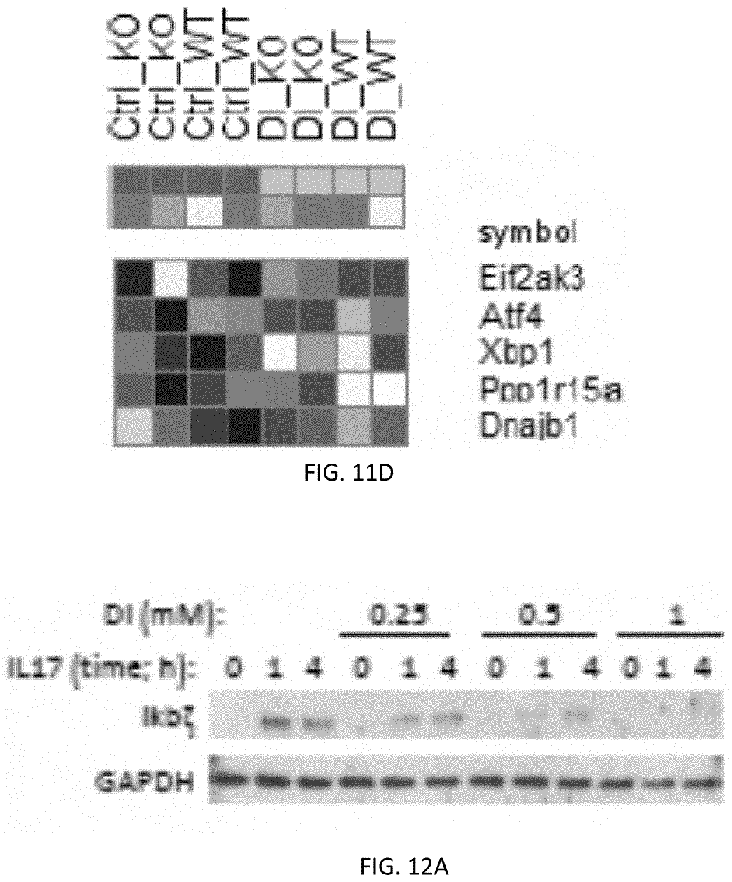

FIG. 11 (A) Ikbζ expression in BMDMs treated with 250 μM DI for 12 h and stimulated with LPS. In some samples N-acetylcysteine was added simultaneously with DI (NAC_12) or at the time of LPS stimulation (NAC_0). (B) Ikbζ expression in Nrf2 deficient BMDMs. Cell were treated with 250 μM DI for 12 h prior to LPS stimulation. (C) Transcriptional comparison of DI treated WT and Nrf2 KO BMDM. Left panel shows signature pathways up-regulated and down-regulated by DI in independent of Nrf2. (D) Most significantly up-regulated genes by DI in Nrf2-independent manner.

DI Down-Regulates IL17-Induced Ikbζ Activation in Keratinocytes

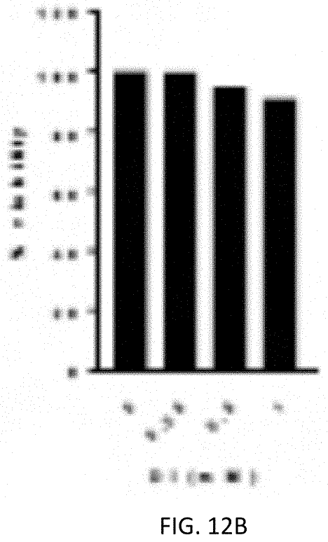

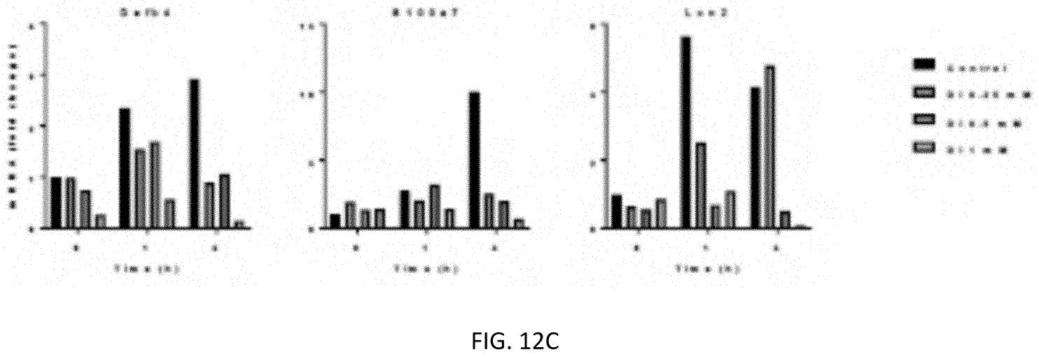

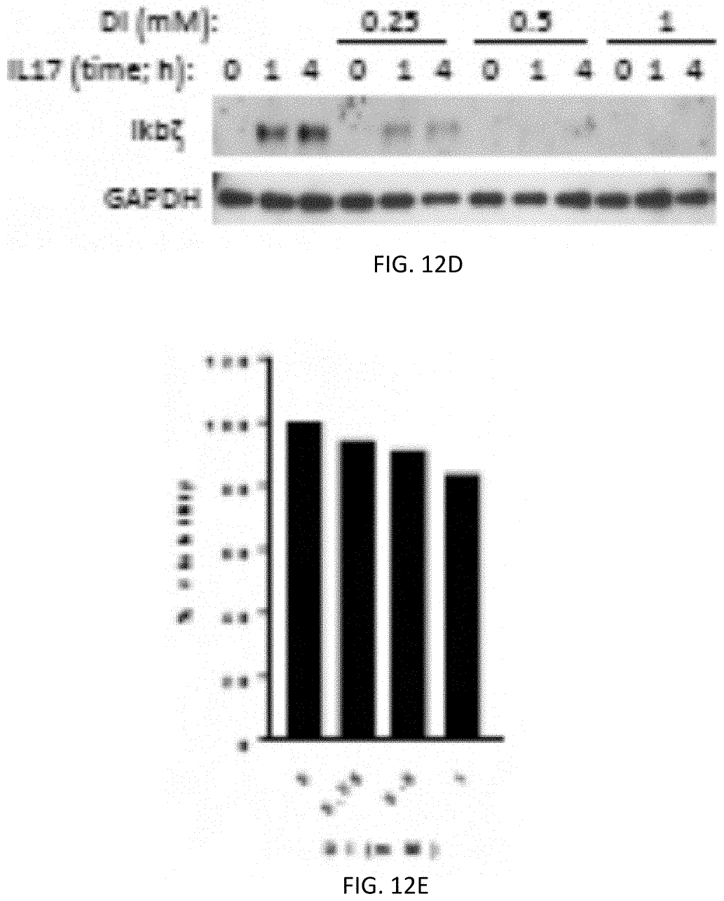

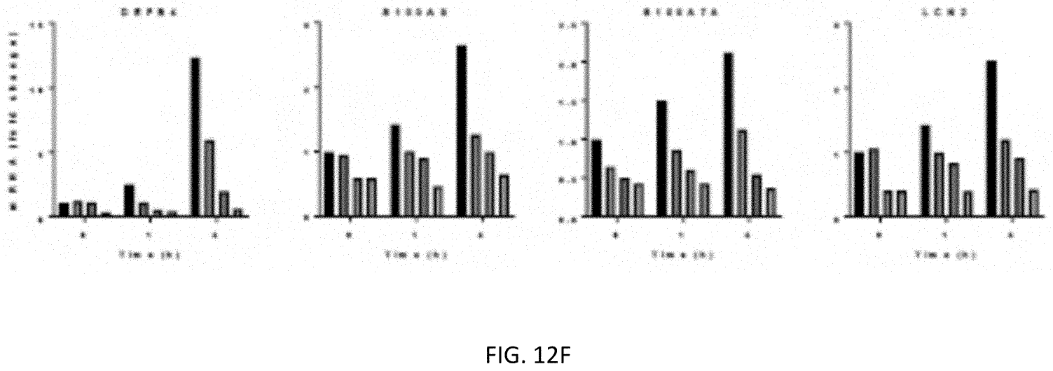

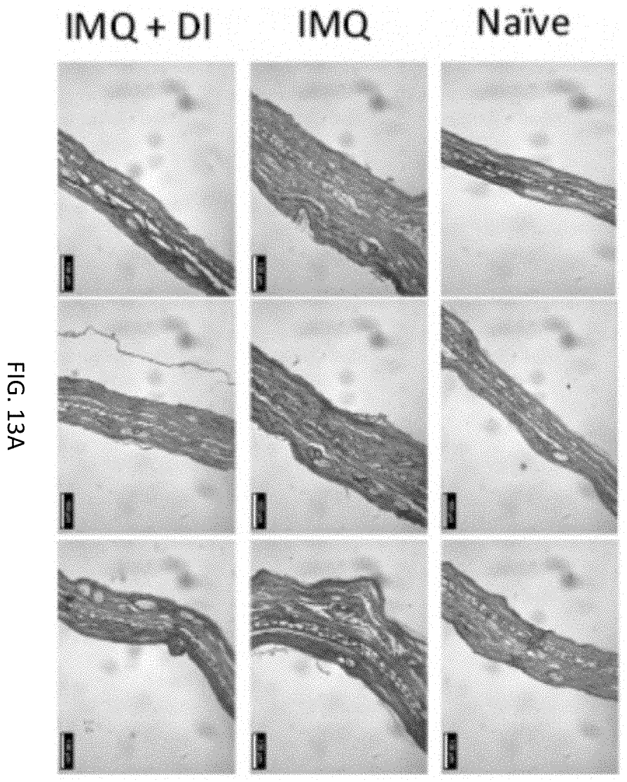

FIG. 12 (A) Ikbζ expression in primary mouse keratinocytes that were treated with DI for 12 h and then stimulated with IL-17A (100 ng/ml). (B) Viability of mouse keratinocytes treated as in (A). (C) qPCR analysis of gene expression in mouse keratinocytes treated as in (A). (D) Ikbζ expression in primary human keratinocytes that were treated with DI for 12 h then stimulated with IL-17A (100 ng/ml). (E) Viability of mouse keratinocytes treated as in (D). (F) qPCR analysis of gene expression in mouse keratinocytes treated as in (D).

DI Ameliorates Psoriatic Pathology in Mouse Model

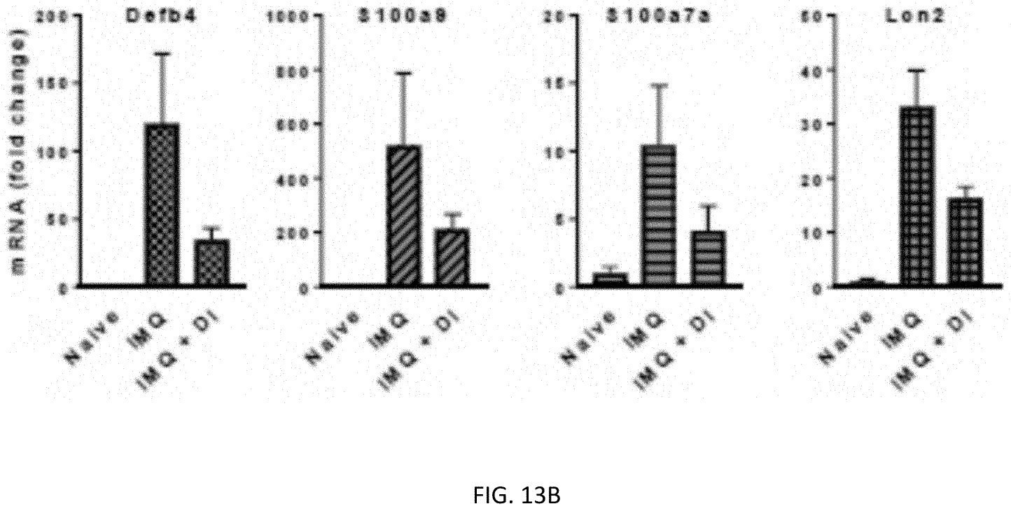

FIG. 13A BIG mice were injected i.p. with a dose of DI and imiquimod (IMQ) was applied topically on ear skin daily for 7 days. Sections of imiquimod-treated ears from mice following 5 d of treatment are shown. FIG. 13B qPCR analysis of gene expression in skin tissue of mice treated as in (A).

### CONCLUSIONS

This example showed that (i) DI triggers Nrf2 antioxidant response, transiently increases cellular ROS, and depletes GSH; (ii) DI down-regulates expression of Ikbζ on posttranscriptional and/or translational level; (iii) the effect of DI on Ikbζ is independent of Nrf2 response; and (iv) DI inhibits IL-17-induced Ikbζ and its target genes in primary keratinocytes and in the model of murine psoriasis.

### Example 2 References

1. Lampropoulou, V., Sergushichev, A., Bambouskova, M., Nair, S., Vincent, E. E., Loginicheva, E., . . . Artyomov, M. N. (2016). Itaconate Links Inhibition of Succinate Dehydrogenase with Macrophage Metabolic Remodeling and Regulation of Inflammation. Cell Metabolism, 24(1), 158-166.
2. McGuire, V. A., Ruiz-Zorrilla Diez, T., Emmerich, C. H., Strickson, S., Ritorto, M. S., Sutavani, R. V., . . . Arthur, J. S. C. (2016). Dimethyl fumarate blocks pro-inflammatory cytokine production via inhibition of TLR induced M1 and K63 ubiquitin chain formation. Scientific Reports, 6(1), 31159.
3. Yamamoto, M., Yamazaki, S., Uematsu, S., Sato, S., Hemmi, H., Hoshino, K., . . . Akira, S. (2004). Regulation of Toll/IL-1-receptor-mediated gene expression by the inducible nuclear protein IκBζ. Nature, 430(6996), 218-222.
4. Johansen, C., Mose, M., Ommen, P., Bertelsen, T., Vinter, H., Hailfinger, S., . . . Iversen, L. (2015). IκBζ is a key driver in the development of psoriasis. Proceedings of the National Academy of Sciences of the United States of America, 112(43), E5825-33.

### Example 3: Electrophilic Stress Induced by Itaconate and its Derivatives Regulates IKBζ/Atf3 Inflammatory Axis

Metabolic regulation emerged as a novel powerful principle guiding immune responses. Inflammatory macrophages undergo extensive metabolic rewiring marked by significant production of itaconate that was recently described as an immunoregulatory metabolite. Itaconate and its membrane permeable derivative dimethyl itaconate (DI) selectively inhibit a subset of cytokines, affecting IL-6, IL-12 but not TNF-α. The major effects of itaconate on cellular metabolism during macrophage activation has been mainly accounted for the inhibition of succinate dehydrogenase (SDH). Yet, SDH inhibition alone is not sufficient for the striking immunoregulatory effects observed with DI. Furthermore, the regulatory pathway responsible for such selective effects of itaconate/DI on inflammatory program has not been defined. Here it is shown that itaconate/DI induces electrophilic stress, reacts with glutathione and subsequently induces both Nrf2-dependent and Nrf2-independent responses. It was found that electrophilic stress can selectively regulate secondary but not primary transcriptional response to toll-like receptor (TLR) stimulation via inhibition of IκBζ protein induction. IκBζ regulation occurs in an Nrf2-independent manner, and ATF3 was identified as its key mediator. This inhibitory effect is conserved across species and cell types, and DI administration in vivo ameliorates IL-17/IκBζ-driven skin pathology in the mouse model of psoriasis, highlighting therapeutic potential of this regulatory pathway. The present results demonstrate that targeting the DI-IκBζ regulatory axis can serve as an important new strategy for the treatment of IκBζ-mediated autoimmune diseases.

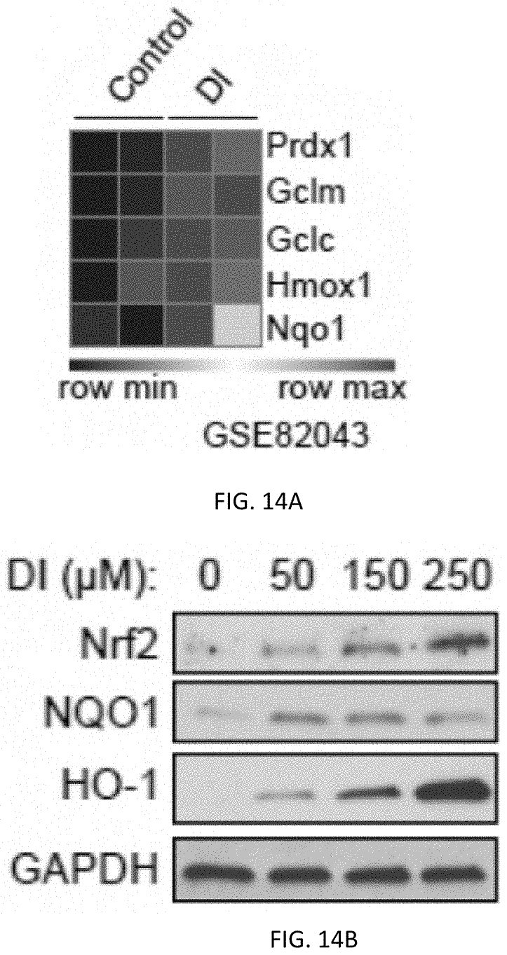

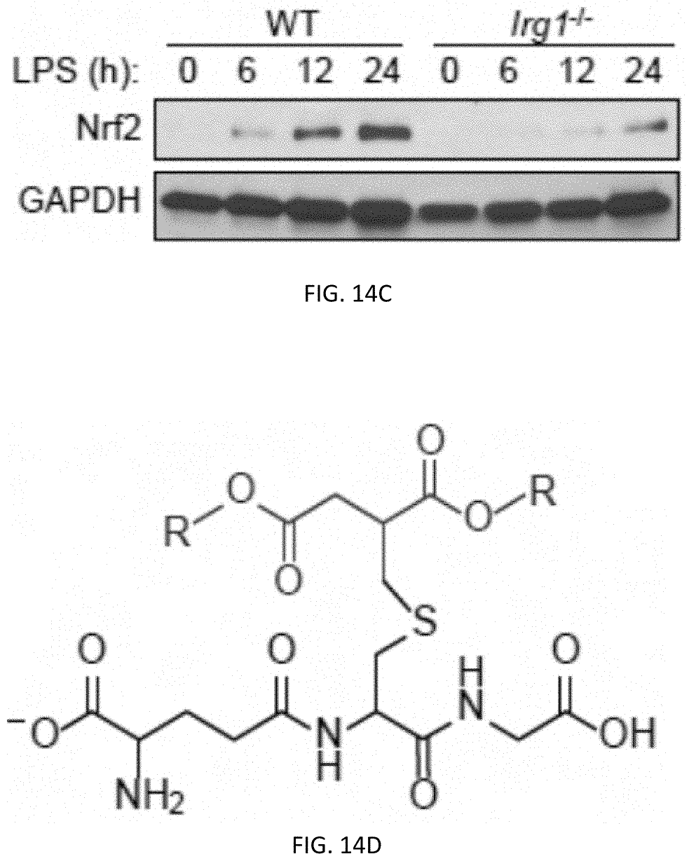

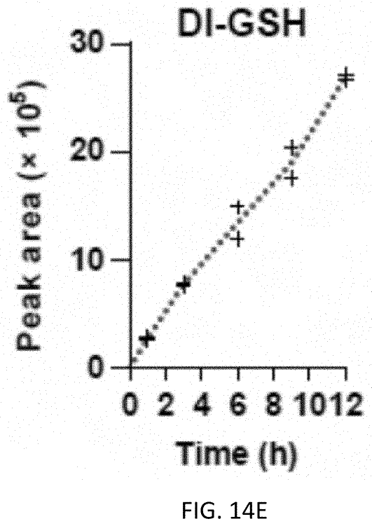

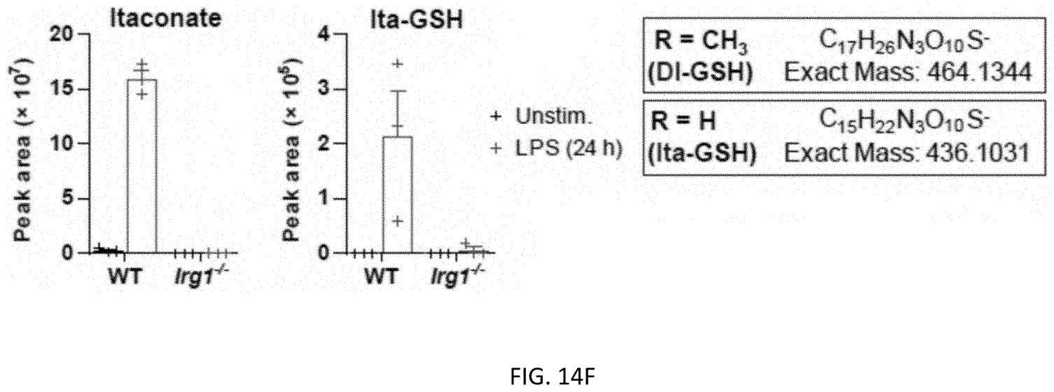

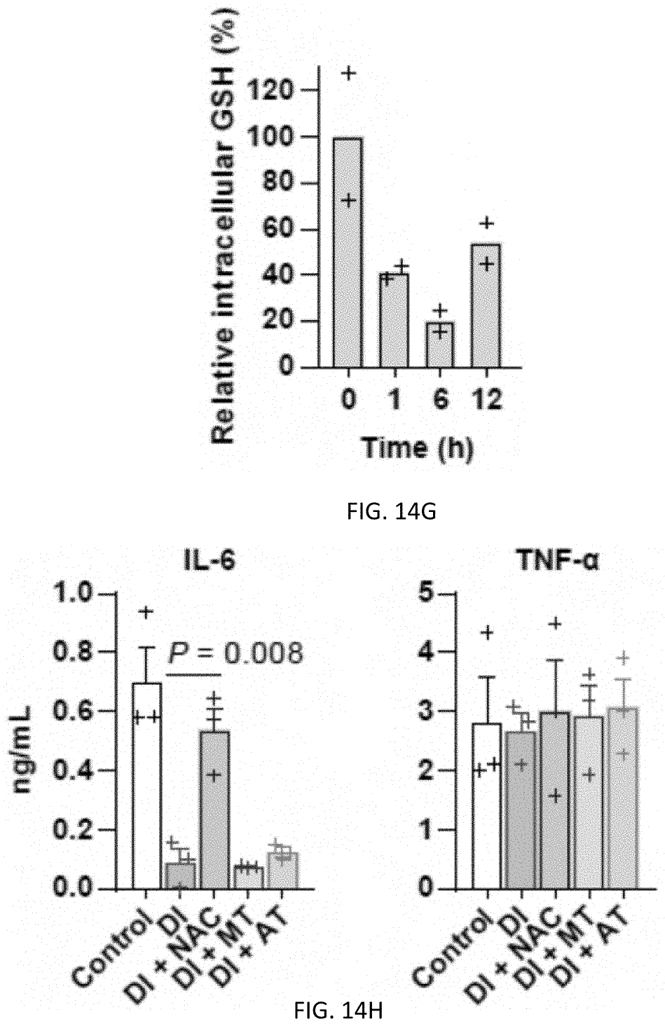

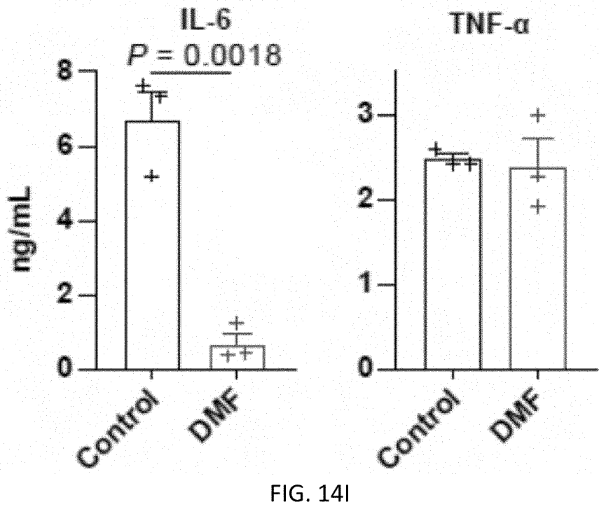

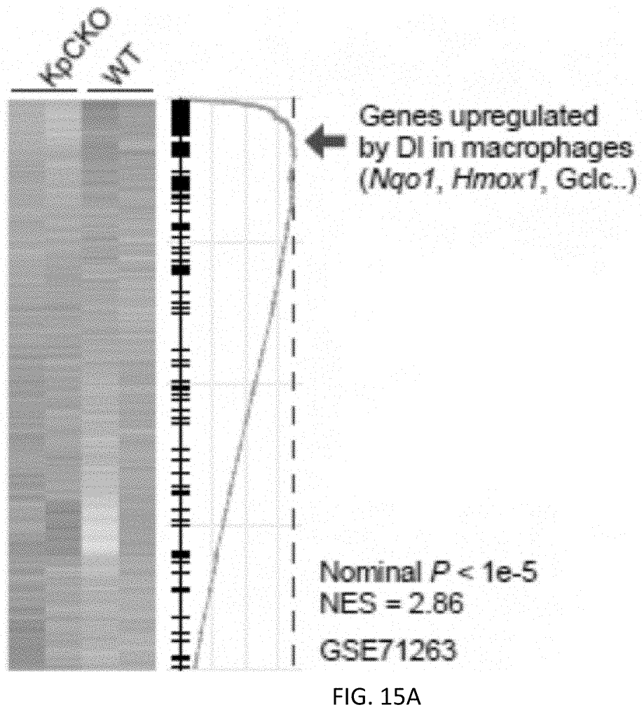

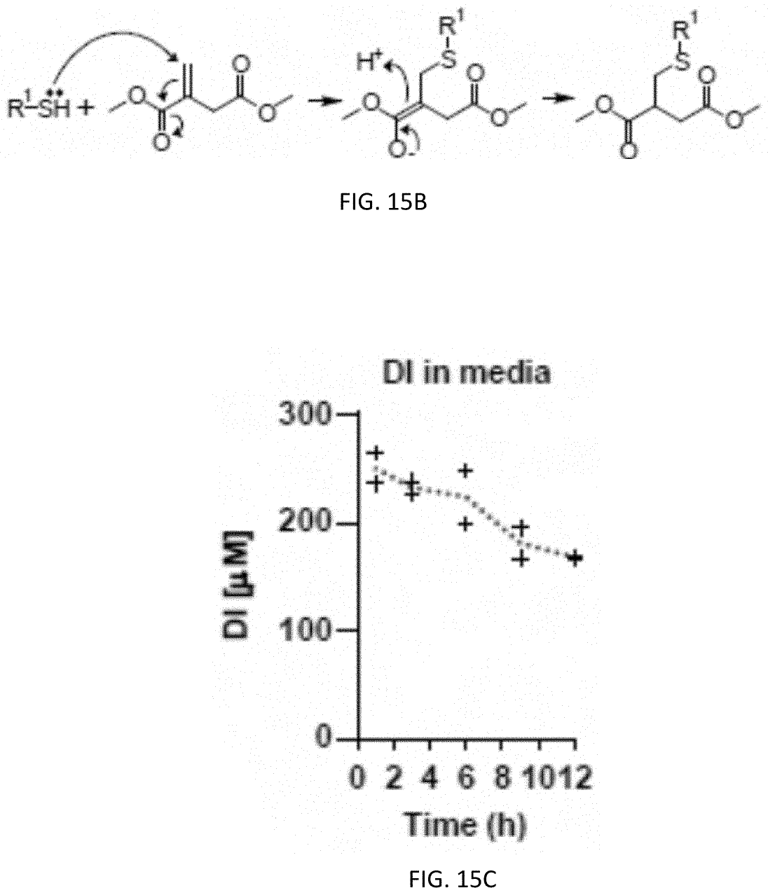

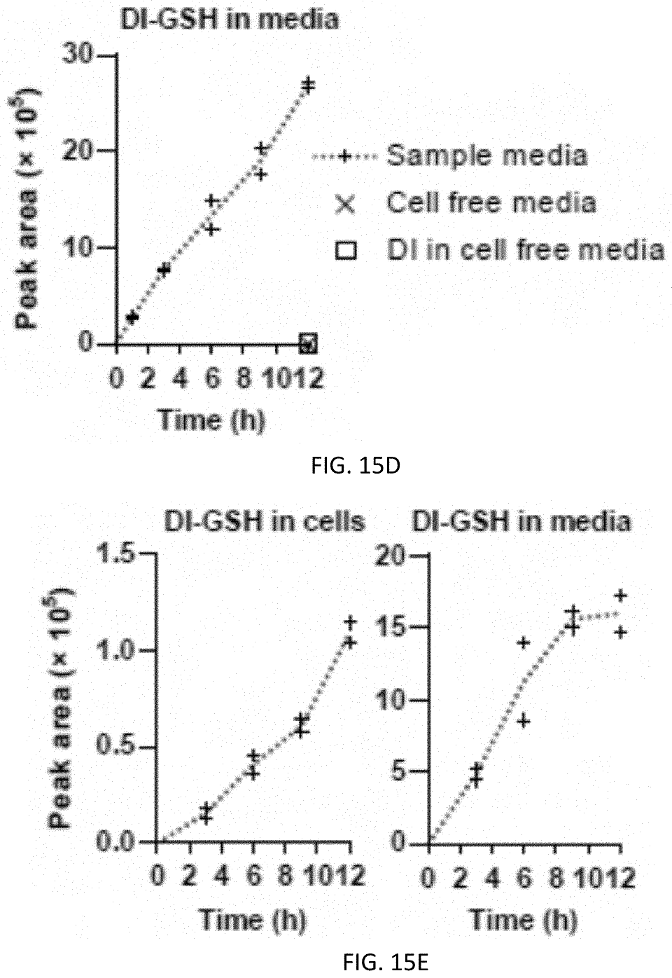

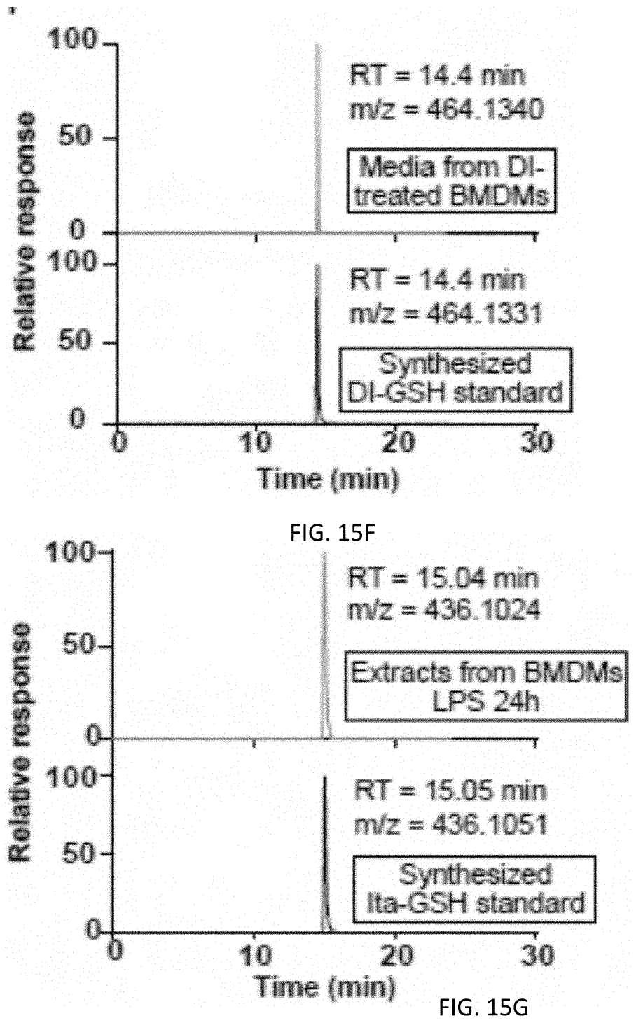

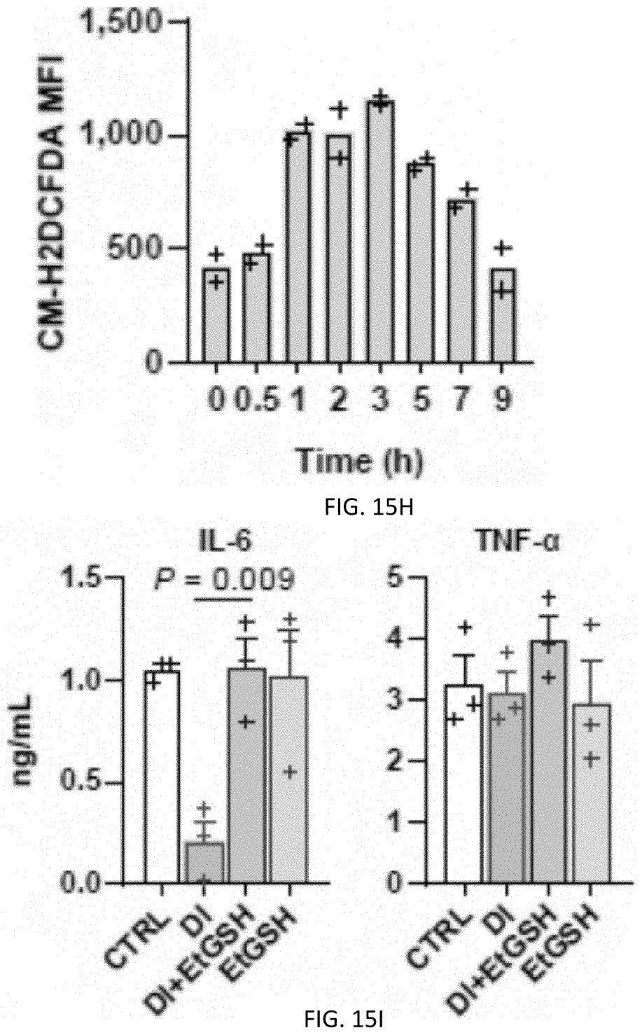

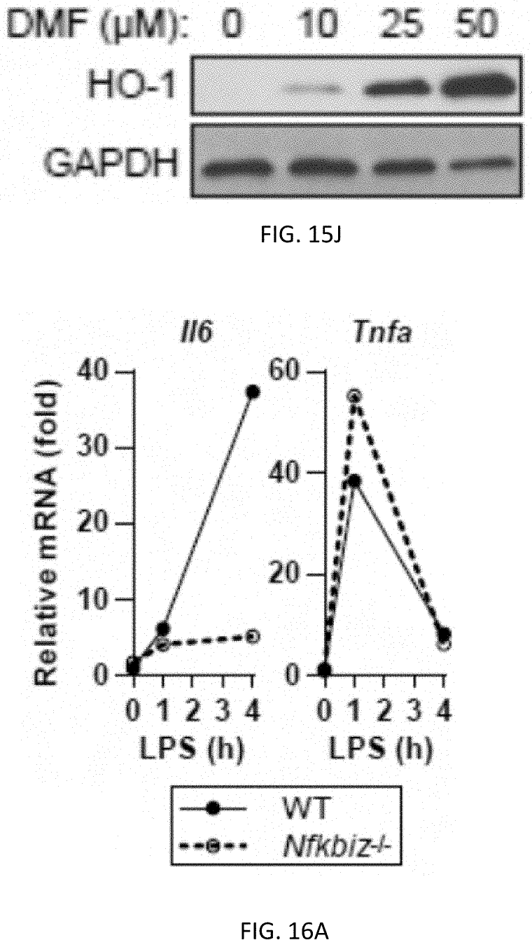

Differential gene expression in the previously described transcriptional analysis of DI-treated bone-marrow derived macrophages (BMDMs) showed enrichment for electrophilic/xenobiotic stress response pathways, specifically upregulation of classical transcriptional markers of Nrf2-mediated response Hmox1, Nqo1, Gclm etc. (see e.g., FIG. 14A). Indeed, Nrf2 protein levels as well as the protein levels of its classical target genes increased during 12 hour DI treatment (see e.g., FIG. 14B). Endogenous itaconate also induced Keap1-Nrf2 response, as Nrf2 protein levels upon LPS activation were higher in WT macrophages than in Irg1−/− (see e.g., FIG. 14C). Overall, transcriptional signatures of DI treatment matched those of the Keap1 knockout macrophages (see e.g., FIG. 15A) indicating induction of global electrophilic stress response.

Given its structure, DI can readily act as an electrophile in Michael reaction (see e.g., FIG. 15B) and trigger electrophilic stress, which is commonly controlled via glutathione (GSH) buffering. For instance, covalent conjugation with GSH was described forfumarate, a metabolite typically accumulated in cells with mutated fumarate hydratase. Analysis of the cell media from DI-treated macrophages suggested that it was uptaken from media and showed significant peak at retention time 14.4 min with the m/z of 464.1334 that was accumulating during incubation with DI and corresponded to diester of methylsuccinated GSH (DI-GSH) (see e.g., FIG. 14D, FIG. 14E, FIG. 15C, and FIG. 15D). DI-GSH origin was confirmed using synthesized 13C5-labeled DI for cell treatment (see e.g., FIG. 15E). Importantly, the reactivity with GSH was also observed for natural itaconate; methylsuccinated GSH (Ita-GSH, see e.g., FIG. 14D) was detected in LPS-stimulated macrophages that correlated with itaconate production and was absent in Irg1−/− cells (see e.g., FIG. 14F). The identity of the DI-GSH and Ita-GSH conjugates was confirmed by LCMS retention time with a chemically synthesized standards (see e.g., FIG. 15F and FIG. 15G).

The reactivity of DI led to substantial drop of cellular GSH concentration and associated increase in ROS generation (see e.g., FIG. 14G and FIG. 15H). Thus, a panel of antioxidants/ROS scavengers was tested by administering them simultaneously with DI. Only N-acetylcysteine (MAC) or cell permeable GSH (EtGSH) co-treatment reversed the effect of DI suggesting that DI acts preferentially by modulating the cellular pool of thiol-containing molecules (see e.g., FIG. 14H and FIG. 15I). Prototypical electrophile dimethyl fumarate (DMF) also triggered Nrf2 response and showed selective inhibition of IL-6 (see e.g., FIG. 14I and FIG. 15J).

In fact, TNFA is induced during primary transcriptional response to TLR stimulation, while II6 is a product of the secondary transcriptional responses. The major transcription factor that was reported to selectively regulate secondary transcriptional response to TLR activation is IκBζ, encoded by the Nfkbiz gene (see e.g., FIG. 16A, FIG. 17A, and FIG. 17B). Strikingly, DI completely abolished LPS-induced induction of IκBζ protein and inhibited its target genes (II12b, Edn1, etc.) in both BMDMs and human blood monocytes (see e.g., FIG. 16B, FIG. 17C, FIG. 17D, FIG. 17E, FIG. 17F, and FIG. 17G). This suggested that observed specificity of DI action on IL-6 may stem from the selective inhibition of the secondary transcriptional response to TLR activation. Indeed, DI did not inhibit IκBα degradation and upstream signaling in response to LPS nor did it prevent LPS-mediated p65 nuclear translocation (see e.g., FIG. 17H, FIG. 17I, and FIG. 17J). Whether NAC/EtGSH-mediated reversal of the DI effect on IL-6 was associated with recovery of IκBζ protein induction was then tested. Indeed, co-treatment of BMDMs with DI and NAC (or EtGSH), but not α-tocopherol, restored LPS-mediated IκBζ induction in BMDMs and in human blood monocytes, while NAC itself was not able to elevate IL-6 levels on untreated or DI-treated Nfkbiz−/− background BMDMs (see e.g., FIG. 16C, FIG. 17K, FIG. 17L, and FIG. 17M). Consistent with the notion that primary NF-κB-mediated response is not affected by DI at this concentration range, Nfkbiz mRNA levels were not changed by DI, affecting IκBζ protein induction at the post-transcriptional level (see e.g., FIG. 16D, FIG. 16E, and FIG. 18A).

To understand the regulation of IκBζ protein proteasome, inhibitor MG132 and autophagosome-lysosome fusion inhibitor bafilomycin A was used, but neither rescued IκBζ induction during DI treatment (see e.g., FIG. 18B). DI action was also not due to regulation of Nfkbiz 3′UTR as indicated by GFP reporter assay (see e.g., FIG. 18C). Finally, cellular stress had been shown to regulate mRNA translation via phosphorylation-driven inactivation of the eIF2a. Indeed, a marked increase in macrophage eIF2α phosphorylation was detected in response to DI (see e.g., FIG. 18 D) suggesting that the inhibitory effect of DI on IκBζ protein levels is associated with regulation of IκBζ translation. Yet, this suppression was very specific to IκBζ, and possibly few other proteins, as DI did not affect protein synthesis globally judged by either metabolic labeling of nascent proteins or proteomic profiling of DI treated macrophages (see e.g., FIG. 18E, FIG. 18F, FIG. 18G, and FIG. 18H).

To further understand the effect of electrophilic stress on IκBζ-mediated inflammatory program, a panel of itaconate derivatives with different levels of electrophilicity and also included DMF (see e.g., FIG. 16F) was set up. Inhibitory effects of compounds on IκBζ correlated with their electrophilic strength: DI, DMF and 3MI inhibited IκBζ induction while MI did not (see e.g., FIG. 16G and FIG. 16H). To decouple contribution of the feedback GSH synthesis during electrophilic stress response to these compounds, combinations of itaconate derivatives with buthionine sulfoximine (BSO) were tested, a non-electrophilic inhibitor of GSH synthesis was tested, which itself does not inhibit cytokine production nor trigger Nrf2 response (see e.g., FIG. 19A, FIG. 19B, and FIG. 19C). BSO enhanced the inhibitory effect of DI on IκBζ/IL-6 (see e.g., FIG. 16I and FIG. 19D) and unlocked the effect of MI on IκBζ/IL-6 (see e.g., FIG. 16J and FIG. 19E)

Effects of endogenously-produced itaconate (weak electrophile) on the IκBζ-regulatory axis were considered. Temporal dynamics of IκBζ upon LPS activation is very different from that of itaconate: IκBζ is peaking at around 1 hour and is already downregulated significantly by 4 hours, while itaconate is only getting induced at 2-4 hours and plateaus later after LPS stimulation (see e.g., FIG. 16K). Thus, to address the physiological relevance of endogenous itaconate to IκBζ regulation, an experiment was designed where both natural itaconate and IκBζ are present in the cells at the same time. Specifically, in vitro macrophage tolerization was evaluated, when cells are first stimulated with LPS and then rechallenged with LPS at 18 hours. This design ensures that endogenous itaconate is produced in sufficient amounts and LPS restimulation triggers IκBζ induction once again. It was observed that in the presence of BSO, there was striking difference in IκBζ protein levels upon restimulation between Irg1−/− and WT cells (see e.g., FIG. 16L and FIG. 19F), confirming potential regulatory effects of natural itaconate on IκBζ.

Next, major regulatory hubs connecting the electrophilic stress to the blockade of IκBζ induction was identified. First, whether Nrf2 is involved in the DI inhibition of IκBζ synthesis was tested. Nrf2-deficient BMDMs did not alleviate DI-mediated IκBζ and IL-6 inhibition (see e.g., FIG. 20A, FIG. 20B, and FIG. 21A). Similarly, macrophages genetically lacking p62/SQSTM1 or HO-1 did not rescue IκBζ inhibition by DI (see e.g., FIG. 21B, FIG. 21C, and FIG. 21D). Given the Nrf2-independent nature of DI action, RNA-seq in Nrf2−/− and WT BMDMs were performed and the genes that were differentially expressed upon DI treatment were analyzed (see e.g., FIG. 21E). Most differentially DI-regulated pathways included upregulated integrated stress response pathways (Atf3, Atf4 and Eif2ak3/PERK etc.), while downregulating interferon (IFN) response pathway (Isg15 etc.) (see e.g., FIG. 20C and FIG. 21F). Focusing on ATF3 as a potential candidate, Nrf2-independent transcriptional signature of DI treatment was compared to the publicly available dataset that profiled WT and Atf3−/− macrophages at their basal states. Strikingly, highly statistically significant overlap between genes regulated by ATF3 and genes regulated by DI were found (see e.g., FIG. 20D), suggesting that DI action might be mediated by ATF3. Indeed, ATF3 protein was upregulated by DI treatment even on Nrf2−/− background (see e.g., FIG. 20E). Furthermore, Atf3−/− cells restored IκBζ protein levels upon DI treatment and significantly increased IL-6 production in DI-treated Atf3−/− cells compared to WT (see e.g., FIG. 20F and FIG. 20G). In Atf3−/− macrophages, DI failed to increase eIF2α phosphorylation, even though it still induced Nrf2 response (see e.g., FIG. 21G and FIG. 21H). DI-mediated ATF3 expression was efficiently decreased by co-treatment with NAC or EtGSH in both mouse macrophages and human monocytes (see e.g., FIG. 21I, FIG. 21J, and FIG. 21K). Endogenous itaconate also efficiently induced ATF3 response: when comparing WT and Irg1−/− BMDMs tolerized in the presence of BSO and then restimulated, ATF3 was induced in WT cells but not in the Irg1−/− (see e.g., FIG. 20H)

Intriguingly, IκBζ also plays major role outside the macrophage context: it is induced upon IL-17 treatment of epithelial cells and orchestrates downstream inflammatory responses. In fact, Nfkbiz polymorphisms have been associated with a number of immune related conditions, including psoriasis. Thus, the in vitro effect of DI pretreatment on IκBζ induction in IL-17A-stimulated primary keratinocytes was first tested. Induction of IκBζ was inhibited by DI in primary mouse and human keratinocytes (see e.g., FIG. 22A, FIG. 22B, and FIG. 23). To further examine the DI inhibitory effect on IκBζ induction by IL-17, expression of the well characterized IκBζ target genes such as Defb4, S100a7a, Lcn2 and S100a9 in mouse and human keratinocytes were analyzed. As expected, expression of these genes was downregulated by DI in correlation with IκBζ protein levels (see e.g., FIG. 22C and FIG. 22D). These data suggested that DI can also modulate induction of IκBζ in multiple immune contexts.

Thus, the ability of DI to interfere with IκBζ signaling in vivo was explored. A mouse model of psoriasis induced by the TLR7/8 agonist imiquimod (IMQ) was used. In this model, skin inflammation is induced by topical application of IMQ cream on mouse ears to induce psoriasis-like pathology resembling the human disease. Daily topical application of IMQ to the mouse ear skin for 7 days led to significant scaling and edema of the skin in control animals while mice treated with DI in addition to IMQ manifested no detectable skin changes (see e.g., FIG. 22E, FIG. 22F, and FIG. 22G). Likewise, quantitative analysis of ear skin-derived mRNA showed significant induction of the IκBζ target genes Defb4, S100a9, S100a7a, and Lcn2 after IMQ application, whereas their expression was markedly reduced in the skin of mice treated with DI (see e.g., FIG. 22H). Daily DI administration did not significantly affect SDH activity in heart and liver (see e.g., FIG. 24A, FIG. 24B, and FIG. 24C), suggesting favorable safety profile. These data demonstrate that DI can act as an IκBζ inhibitor in vivo and could provide a targeted approach for treating various autoimmune conditions.

Experimental Animals

C57BL/6N WT were from Charles River Laboratories. Irg1−/− mice were previously published. Nrf2−/− mice (cat #017009) and control WT C57BL/6J mice (cat #000664) were purchased from Jackson Laboratory. Nfkbiz−/− mice described were provided by Prof. Shizuo Akira, sex matched animals were used in experimnets. p62-deficient mice were provided by Prof. Herbert W. Virgin (Department of Pathology and Immunology, Washington University School of Medicine, USA). Mice were maintained at Washington University under specific pathogen-free conditions in accordance with Federal and University guidelines and protocols approved by the Animal Studies Committee of Washington University. Femurs and tibias from Hmox1lox/− and control LyzMcre/creHmox1lox/− described were provided by Dr. Miguel P. Soares. Femurs and tibias from Atf3−/− mice as described and control C57BL/6 WT mice were provided by Dr. Tsonwin Hai. Mice used for the study were 6-12 weeks old. If not indicated otherwise, female mice were used.

Bone Marrow-Derived Macrophages (BMDMs) and Mouse Cell Cultures

BMDM were prepared from 6- to 12-week-old mice as described 1 and cultured in RMPI-1640 medium supplemented with 10% fetal bovine serum (FBS), 2 mM L-glutamine, and 100 U/mL penicillin-streptomycin and mouse recombinant M-CSF (20 μg/mL, Peprotech). For experiments, cells were seeded at concentration 106 cells/mL in tissue culture plates of various formats. The cells were treated with DI (250 μM (unless stated otherwise), cat #592498, Sigma), DMF (50 μM (unless stated otherwise), cat #242926, Sigma), 3-(ethoxycarbonyl)but-3-enoic acid (3MI, 5 μM, Aris Pharmaceuticals Inc.), 4-ethoxy-2-methylene-4-oxobutanoic acid (MI, Aris Pharmaceuticals Inc.) for 12 h and activated with lipopolysaccharide (LPS; 100 ng/mL; E. coli 0111:B4, Sigma) for 1 h or as indicated. In some experiments cells were stimulated with combination of LPS (20 ng/mL) and IFN-γ (50 ng/mL; Peprotech). In some experiments cells were treated with α-tocopherol (AT, 10 μM; Sigma), MitoTEMPO (MT, 500 μM; Sigma), N-acetylcysteine (NAC, 1 mM, Sigma), ethylester of GSH (EtGSH; 1 mM, Santa Cruz) or buthionene sulfoximine (BSO, 500 μM, Sigma) alone or in combination with DI for 12 h. In some experiments BMDMs were treated with DI for 12 h and bafilomycin A (BafA, 100 nM, Sigma) or MG132 (10 μM, Selleckchem) were added 30 min before subsequent LPS stimulation.

In tolerization experiments cells were stimulated with LPS (100 ng/ml) for 18 h then washed with PBS at 37° C. and restimulated with LPS (100 ng/ml) for 1 h. In some samples, BSO (500 μM) was present during first stimulation.

BV2 microglial cell line was a kind gift from Prof. Herbert W. Virgin (Department of Pathology and Immunology, Washington University School of Medicine, USA). BV2 cells were maintained in DMEM medium supplemented with 10% FBS, 2 mM L-glutamine, 1 mM sodium pyruvate and 100 U/mL penicillin-streptomycin.

RNA Sequencing (RNA-Seq) Analysis

mRNA was extracted with oligo-dT beads (Invitrogen), and libraries were prepared and quantified as described. All RNA-seq experiments generated in the study were performed in n=2 independent cultures. Raw and processed data was deposited to Gene Expression Omnibus with accession numbers GSE102190 and GSE110749. Pre-ranked gene set enrichment analysis (GSEA) was done using fgsea R package. For analysis of WT and Nrf2−/− BMDMs genes were ranked according signal to noise statistic, only top 10,000 genes ordered by mean expression were considered. MSigDB C2 and H gene set collections were used. For analysis of Keap1 conditional KO (KpCKO)(GSE71263) and Atf3−/− (GSE61055) datasets, differential expression analysis was carried out using limma package, genes were ranked by the corresponding test statistics and P were calculated using pre-ranked gene set enrichment analysis method fgsea package with 200,000 gene-set permutations. Heatmaps were generated using Phantasus web-service (https://artyomovlab.wustl.edu/phantasus/).

Western Blots

Cells were lysed in RIPA Lysis Buffer System (Santa Cruz) and heat-denatured at 95° C. for 5 min in reducing sample buffer (BioRad). Proteins were separated on 4%-20% polyacrylamide gradient gels (BioRad) and transferred onto 0.45 μm pore size PVDF membranes (Millipore). Non-specific binding was blocked with 5% skim milk (or 5% BSA when phospho-proteins were analyzed), and membranes were probed with primary antibodies specific to Nrf2 (#12721), HO-1 (#70081), IκBζ (mouse specific, #93726), IκBζ(#9244), ATF3 (#D2Y5W), IRAK1 (#4504; sensitivity of IRAK1 detection diminishes upon IRAK1 K63 ubiquitination), phospho-IKK (Ser176/180, #2697), p62/SQSTM1 (#5114), phospho-eIF2α (Ser51, #9721), eIF2α (#5324) from Cell Signaling; GAPDH (sc-25778), IκBα (sc-1643), ATF3 (sc-188), SDHA (sc-166909) from Santa Cruz; NQO1 (ab28947) from Abeam, followed by incubation with anti-rabbit-HRP (1:10,000; sc-2030) or anti-mouse-HRP (1:10,000; sc-2031) from Santa Cruz and Clarity western ECL substrate (Bio-Rad). Membranes were exposed to X-Ray films (Research Products International Corp.) and developed using SRX-101A film processor (Konica Minolta). GAPDH run on the same blot was used as loading control. After scanning of original films image brightness of some blots was adjusted and bands were cropped using ImageJ. Densitometry was performed using ImageJ.

DI Detection with Gas-Chromatography-Mass Spectrometry (GCMS)

For DI measurement media was collected from cells at various time points of incubation with 250 μM DI and placed on ice. An equal volume of ethyl acetate (Sigma) was added and the samples were vortexed at 4° C. for 1 min. After centrifugation at 14,000 g for 2 min at 4° C., the organic phase was collected and approx. 20-30 mg of sodium sulfate (Sigma) was added. The samples were vortexed prior to analysis. GCMS analysis was performed using a Thermo Trace 1300 GC equipped with a 30 m DB-35MS capillary column connected to a Thermo TSQ Quantum MS operating under electron impact (EI) ionization at 70 eV. 1 μL of sample was injected in splitless mode at 270° C., using helium as the carrier gas at a flow rate of 1 mL min-1. The GC oven temperature was held at 100° C. for 3 min and increased to 240° C. at 3.5° min-1. The MS source and quadrupole were held at 230° C. and 280° C., respectively, and the detector recorded ion abundance in the range of 30-800 m/z.

Metabolite Profiling with Liquid Chromatography-Mass Spectrometry (LCMS)

Bone marrow-derived macrophages were seeded in 96-well plates at 105 cell per well for all analyses. After treatment, media was removed from the wells and the cells were washed 3× with PBS (37° C.) and immediately placed on dry ice. The frozen cells or media were stored at −80° C. until extraction. Cell extracts were prepared by adding 180 μL of 70/30 Ethanol/H2O solution at 70° C. with 300 ng/mL 13C5 15N1 d5-Glutamate as the internal standard. After rigorous mixing, the supernatant was collected after centrifugation (21,694 g for 10 min at 4° C.) and transferred to another 96-well plate and the solvent was evaporated under reduced pressure (Genevac). Prior to injection, dried extracts were reconstituted, or media was diluted (1:20) in LCMS grade water. The extracted samples were analyzed by high-resolution accurate mass (HRAM) liquid-chromatography-mass spectrometry. LC separation was achieved by reverse-phase ion-pairing chromatography. The UHPLC system consisted of a Vanquish (Thermo Fisher Scientific, San Jose, USA) pumping system, coupled to an autosampler and degasser. Chromatographic separation was performed using a Synergy Hydro-RP column (100 mm×2 mm, 2.5 μm particle size, Phenomenex, Torrance, Calif.). The elution gradient was carried out with a binary solvent system as described previously. HRAM data was acquired using a QExactive™ Orbitrap mass spectrometer (Thermo Fisher Scientific), which was equipped with a heated electrospray ionization source (HESI-II), operated in negative electrospray mode. Ionization source working parameters were optimized; the heater temperature was set to 300° C., ion spray voltage was set to 3500 V. An m/z scan range from 70 to 700 was chosen and the resolution was set at 70,000. The automatic gain control target was set at 1e6 and the maximum injection time was 250 ms. Instrument control and acquisition was carried out by Xcalibur 2.2 software (Thermo Fisher Scientific). All data analysis was conducted using EL-MAVEN software.

### Synthesis of DI-GSH Conjugate (N5-(1-((carboxymethyl)amino)-3-((4-methoxy-2-(methoxycarbonyl)-4-oxobutyl)thio)-1-oxopropan-2-yl)glutamine)

To a vial charged with dimethyl 2-methylenesuccinate (0.158 g, 1.0 mmol) was added ethanol (1.000 mL), triethylamine (0.167 mL, 1.200 mmol) and the mixture was cooled in an ice water bath prior to the addition of GSH (0.369 g, 1.200 mmol). The resulting suspension was stirred overnight and allowed to slowly warm to room temperature (ice melt) affording a light yellow solution. The mixture was dried under reduced pressure and purified by RP-HPLC: Instrumentation: Agilent Automated Purification System w/Single Quad MS and DAD; Waters AcQuity UPLC I-Class with QDa and UV. Method: XSelect CSH Prep C18 OBD 5 μm 19×100 column. Solvents A and B are water w/0.1% formic acid and acetonitrile, respectively. 10 minute method time with a gradient from 10% B to 40% B over 5 minutes. Samples were loaded at 10% B. The flow rate during the loading was 25 mL/min and it was raised to 40 mL/min during separation affording N5-(1-((carboxymethyl)amino)-3-((4-methoxy-2-(methoxycarbonyl)-4-oxobutyl)thio)-1-oxopropan-2-yl)glutamine as a white solid (314 mg, 67.4%) ESI m/z (M+H)+=466.0 1H NMR (400 MHz, DMSO-d6) δ 8.70 (t, J=6.6 Hz, 1H), 8.32 (d, J=8.6 Hz, 1H), 8.13 (d, J=1.2 Hz, 1H), 4.43-4.33 (m, 1H), 3.67 (m, 1H), 3.60 (s, 3H), 3.58 (s, 3H), 3.25 (m, 3H, under water in DMSO), 3.00-2.70 (m, 3H), 2.70-2.56 (m, 3H), 2.28 (m, 2H), 1.95-1.75 (m, 2H).

### Synthesis of Ita-GSH Conjugate (2-(((2-(4-amino-4-carboxybutanamido)-3-((carboxymethyl)amino)-3-oxopropyl)thio)methyl)succinic acid)

To a vial charged with 2-methylenesuccinic acid (itaconic acid) (0.021 g, 0.163 mmol) was added water (0.651 mL) and GSH (0.05 g, 0.163 mmol). The resulting suspension was heated at 37° C. overnight affording a pale yellow solution which was directly purified by RP-HPLC as follows: XSelect Prep C18 5 μm 19×100 column. Solvents A and B are water w/0.1% formic acid and acetonitrile, respectively with a 10 minute method time and a gradient from 5% B to 10% B over 5 minutes. Samples were loaded at 5% B. The flow rate during the loading and the separation was 40 mL/min. Mass spectral data were acquired from 200-1000 amu in electrospray positive mode. Product was successfully isolated, 2-(((2-(4-amino-4-carboxybutanamido)-3-((carboxymethyl)amino)-3-oxopropyl)thio)methyl)succinic acid (28.4 mg, 0.065 mmol, 39.9% yield) as a pale white solid. ESI m/z (M+H)+=437.1 1H NMR (400 MHz, Deuterium Oxide) δ 4.48 (ddd, J=8.7, 5.0, 3.3 Hz, 1H), 3.86 (s, 2H), 3.71 (t, J=6.3 Hz, 1H), 2.97 (ddt, J=12.1, 9.1, 5.6 Hz, 2H), 2.87-2.52 (m, 5H), 2.43 (td, J=7.5, 5.4 Hz, 2H), 2.06 (q, J=7.3 Hz, 2H).

### Synthesis of 13C5-dimethyl itaconate (13C5-DI)

E. coli ita23 (provided by Klamt Lab, Max Planck Institute) was allowed to grow in 12C-glucose-LB broth (10 mL of 10 g/L bacto-trypsin, 5 g/L yeast extract, 10 g/L NaCl, 0.28 g/L CaCl2, 125 mg/L kanamycin and 0.2% (w/v) of 12C-glucose) overnight at 30° C. and 210 rpm (until OD420: 2.6). The production of itaconic acid was next initiated by dilution of 100 μL of the above culture into 250 mL of a 13C-glucose minimal media (5.0 g/L K2HPO4, 3.5 g/L KH2PO4, 3.5 g/L (NH3)NaHPO4, 0.25 g/L MgSO4, 11.3 mg/L CaCl2, 1.5 g/L glutamic acid, 0.5 mg/L thiamine, 25 mg/L kanamycin, 1 mL trace element solution and 0.4% 13C-glucose). The trace element solution consists of 1.6 g/L FeCl3, 0.2 g/L CoCl2 6H2C, 0.1 g/L CaCl2, 0.2 g/L ZnCl2 4H2C, 0.2 g/L NaMoO4, 0.05 g/L H3BC3. The bacteria were allowed to grow for 6 days until the OD420 reached 1.9-2.1. The cells were pelleted by centrifugation for 30 min at 14,000 g and 4° C. The supernatant was collected and lyophilized to afford a white powder (3.78 g). The powder was re-dissolved in distilled H2C (10 mL) and the pH reduced to 2 using concentrated HCl (ca. 0.5 mL). 13C-labelled itaconate was extracted into ethyl acetate (4×15 mL). The combined organic layers were dried over anhydrous Na2SO4 and concentrated in vacuo to afford 13C-labelled itaconate as an off-white solid (283 mg, 1.1 g/L of culture). 1H NMR (d6-DMSO, 500 MHz) δ 12.38 (br s, 2H, —COOH), 6.30-5.92 (m, 1H, H-2a), 5.71 (ddd, 1H, 1JHC: 158.8 Hz, 2JHC: 12.0 Hz, 3JHC: 5.9 Hz, H-2b), 3.38-3.03 (m, 2H, H-4); 13C NMR (d6-DMSO, 125 MHz) δ 172.0 (dt, 1JCC: 55.7 Hz, 2JCC: 2.6 Hz, C-1), 167.5 (d, 1JCC: 68.8 Hz, C-5), 135.3 (tdd, 1JCC: 69.1 Hz, 1 JCC: 46.7 Hz, 2JCC: 2.8 Hz, C-3), 127.4 (d, 1 JCC: 70.8 Hz, C-2), 38.1-36.5 (m, C-4) HR-MS 13C5H6C4 (M+Na+) calcd. 158.0326, found 158.0328.

The reactant 13C-itaconic acid (0.10 g, 9.5 mmol) was dissolved into methanol (1 mL). To this solution was added 1 drop of concentrated H2SO4 and the mixture was refluxed overnight (16 hours). The reaction was quenched with sat. NaHCO3 (1 mL), then extracted into dichloromethane (2×2 mL). The combined organic layers were dried over anhydrous Na2SO4 to afford a brown liquid. Yield: 97 mg, 80%. 1H NMR (CDCl3, 300 MHz) δ 6.62-6.00 (m, 1H, H-2a), 6.00-5.36 (m, 1H, H-2b), 3.74 (d, 3H, 3JHC: 3.8 Hz, H-6), 3.67 (d, 2H, 3JHC: 3.9 Hz, H-7), 3.60-2.99 (m, 2H, H-4); 13C NMR (CDCl3, 75 MHz) δ 172.0-170.6 (m, C-1), 167.5-166.0 (m, C-5), 133.9 (tdd, 1 JCC: 71.9 Hz, 2JCC 46.5 Hz, 3JCC 3.0 Hz, C-3), 130.0-127.2 (m, C-2), 52.4-52.3 (m, C-6), 52.3-52.2 (m, C-1), 38.7-36.7 (m, C-4) HR-MS C213C5H10O4 (M+Na+) calcd. 168.0639, found 168.0638.

Protein Mass Spectrometry

BMDMs were plated 2×106 cells per well in 6-well plates. Cells were either (1) treated with DI (250 μM) for 12 h and then stimulated with LPS (100 ng/mL) for 1 h, or (2) only stimulated with LPS (100 ng/mL) for 1 h, or (3) only treated with DI (250 μM) for 12 h, or (4) neither treated with DI nor LPS. Cells were then washed 3× with PBS and lysed in 200 μL of urea buffer (8 M urea, 75 mM NaCl, 50 mM Tris pH 8.0, 1 mM EDTA). Lysates were then cleared by centrifugation at 20,000 g and protein concentrations were determined by BCA assay (Pierce). 15 μg of total protein per sample were processed further. Disulfide bonds were reduced with 5 mM dithiothreitol and cysteines were subsequently alkylated with 10 mM iodoacetamide. Samples were diluted 1:4 with 50 mM Tris/HCl (pH 8.0) and sequencing grade modified trypsin (Promega) was added in an enzyme-to-substrate ratio of 1:50. After 16 h of digestion, samples were acidified with 1% formic acid (final concentration). Tryptic peptides were desalted on C18 StageTips according to and evaporated to dryness in a vacuum concentrator. Desalted peptides were labeled with the TMTIOplex mass tag labeling reagent according to the manufacturer's instructions (Thermo Scientific) with small modifications. Briefly, 0.2 units of TMTIOplex reagent was used per 15 μg of sample. Peptides were dissolved in 30 μl of 50 mM Hepes pH 8.5 solution and the TMTIOplex reagent was added in 12.3 μl of MeCN. After 1 h incubation the reaction was stopped with 2.5 μl 5% hydroxylamine for 15 min at 25° C. Differentially labeled peptides were mixed for each replicate and subsequently desalted on C18 StageTips and evaporated to dryness in a vacuum concentrator.

The peptide mixtures were fractionated by Strong Cation Exchange (SCX) using StageTips as previously described with slight modifications. Briefly, one StageTip was prepared per sample by 3 SCX discs (3M, #2251) topped with 2 C18 discs (3M, #2215). The packed StageTips were first washed with 100 μl methanol and then with 100 μl 80% acetonitrile and 0.2% formic acid. Afterwards they were equilibrated by 100 μl 0.2% formic acid and the sample was loaded onto the discs. The sample was transeluted from the C18 discs to the SCX discs by applying 100 μl 80% acetonitrile; 0.2% formic acid, which was followed by 3 stepwise elutions and collections of the peptide mix from the SCX discs. The first fraction was eluted with 50 μl 50 mM NH4AcO; 20% MeCN (pH ˜7.2), the second with 50 μl 50 mM NH4HCO3; 20% MeCN (pH 8.5) and the sixth with 50 μl 0.1% NH4CH; 20% MeCN (pH 9.5). 200 μl of 0.2% acetic acid was added to each of the 3 fractions and they were subsequently desalted on C18 StageTips as previously described and evaporated to dryness in a vacuum concentrator. Peptides were reconstituted in 10 μl 0.2% formic acid. Both the unfractionated samples plus the fractionated, less complex samples were afterwards analyzed by LCMS/MS on a Q-Exactive HF was performed as previously described. Briefly, around 1 μg of total peptides were analyzed on an Eksigent nanoLC-415 HPLC system (Sciex) coupled via a 25 cm C18 column (inner diameter of 100 μm, packed in-house with 2.4 μm ReproSil-Pur C18-AQ medium, Dr. Maisch GmbH) to a benchtop Orbitrap Q Exactive HF mass spectrometer (Thermo Fisher Scientific). Peptides were separated at a flow rate of 200 nL/min with a linear 206 min gradient from 2% to 25% solvent B (100% acetonitrile, 0.1% formic acid), followed by a linear 5 min gradient from 25 to 85% solvent B. Each sample was run for 270 min, including sample loading and column equilibration times. Data was acquired in data dependent mode using Xcalibur 2.8 software. MS1 Spectra were measured with a resolution of 60,000, an AGC target of 3e6 and a mass range from 375 to 2000 m/z. Up to 15 MS2 spectra per duty cycle were triggered at a resolution of 60,000, an AGC target of 2e5, an isolation window of 1.6 m/z and a normalized collision energy of 36.

All raw data were analyzed with MaxQuant software version 1.6.0.16 using a UniProt Mus musculus database (downloaded on May/16/2017), and MS/MS searches were performed with the following parameters: The five mass spec runs were grouped together. TMT11plex labeling on the MS2 level, oxidation of methionine, deamidation of asparagine and protein N-terminal acetylation as variable modifications; carbarnidomethylation as fixed modification; Trypsin/P as the digestion enzyme; precursor ion mass tolerances of 20 p.p.m. for the first search (used for nonlinear mass re-calibration) and 4.5 p.p.m. for the main search, and a fragment ion mass tolerance of 20 p.p.m. For identification, a maximum FDR of 1% separately on protein and peptide level was applied. 1 or more unique/razor peptides was required for protein identification and a ratio count for each of the 10 TMT channels. This provided a total of 4123 quantified protein groups.

Finally, the MaxQuant generated corrected TMT intensities were normalized such that at each condition/time point the corrected TMT intensity values added up to exactly 1,000,000, therefore each protein group value can be regarded as a normalized microshare (this was done separately for each TMT channel for all proteins that made the filter cutoff in all the TMT channels). After that a pseudocount of 1 was added to each intensity value in order to account for the noise level and make the fold change calls more robust for small intensity values. Finally, all values were log 2 transformed and the average of the log 2 values for all replicates per condition was established (if replicate samples were present).

Metabolic Labeling of Nascent Protein Synthesis with Click Chemistry

BMDMs were grown in 12-well plate, 106 cells per well and treated with DI (250 μM) for 10 h. The cells were then washed 3× with methionine deficient media and incubated 1 h without methionine in presence of 200 μM DI for 1 h. After that L-azidohomoalanine (Click-iT® AHA, C10102, Invitrogen) was added directly to cell media to final concentration 50 μM. The cells were then stimulated with LPS for 1 h. In some samples, cells were treated with puromycin 5 μg/mL 2 h before LPS stimulation to block translation. Cells were lysed in 100 μL of lysis buffer (50 mM Tris-HCl, pH 8, 1% SDS) supplemented with protease inhibitor cocktail, PMSF and Na3VO4 (Santa Cruz). Lysates were incubated on ice for 30 min, sonicated and cleared by centrifugation 13,000 g for 5 min at 4° C. Total protein concentration was determined using RC/DC™ Protein Assay (Biorad). 30 μg of protein was used for downstream reaction with 40 nM biotin-alkyne (B10185, Invitrogen) and reaction was carried out in Click-iT® Protein Reaction Buffer Kit (C10276, Invitrogen) according to manufacturer's protocol. Proteins were separated on 4%-20% polyacrylamide gradient gels (BioRad) and biotinylated proteins were detected by western blot with streptavidin-HRP conjugate (1:1000, #554066, BD Pharmingen). Membrane was striped using 0.2 M NaOH and reprobed to detect IκBζ (see Western blot analysis section).

Glutathione (GSH) Measurement

Total GSH concentration in cells was determined by GSH/GSSH Ratio Detection Assay Kit (Abeam) according to manufacturer protocol. Briefly, 106 BMDMs were lysed in 100 μL of 0.5% NP-40 in PBS, pH 6. Samples were deproteinized using trichloroacetic acid and neutralized by addition of 1M NaHCO3 to achieve pH 4-6. Collected extracts were diluted with supplied assay buffer and directly used for GSH measurement.

Cytokine Detection

Cytokines in cell supernatants were analyzed using DuoSet® ELISA kits according to manufacturer protocol (R&D Systems). The supernatants from BMDMs were diluted 1:4, from human blood monocytes 1:3.

RNA Isolation and Quantitative Real-Time PCR (qPCR)

RNA from cultured cells was isolated using a Total RNA I kit (OMEGA). RNA from mouse ear skin was extracted using RNAeasy mini kit (Qiagen) after tissue disruption with sterile zirconium beads on a MagNA Lyser (Roche). Isolated RNA was reverse transcribed using AffinityScript Multi-Temp reverse transcriptase (Agilent Technologies) according to the manufacturer's protocol. Reactions were performed in 96-well plates using a SYBR Green PCR Master mix (Thermo Fisher Scientific) using a LightCycler® 96 or LightCycler® 480 (Roche Diagnostics). All assays were performed at least in duplicates, and reaction mixtures in 20 μL volumes (96-plate) or 10 μL volumes (384-plate) were processed under the following cycling conditions: initial 10 min denaturation at 95° C., followed by 40 cycles at 95° C. for 10 s, 60° C. for 1 min. Threshold cycle (CT) values for each sample were determined by automated threshold analysis. Expression levels of all mRNAs were normalized to reference gene Actb in BMDMs or to Rpl19 in mouse keratinocytes and tissue samples; to ACTB in human blood monocytes or RPLP0 in human keratinocytes. The relative increase in the expression level of a gene was normalized to the level of expression in unstimulated control cells in each experiment. Primer pairs used are listed in TABLE 1. IκBζ-dependent genes selected for analysis in macrophages were previously published.

TABLE 1
Primers used in real-time quantitative PR (qPCR).

| | |
|---|---|
| Forward 5′→3′ | Reverse 5′→3′ |

Mouse primers

| | | |
|---|---|---|
| Actin | Ggagggggttgaggtgtt | Tgtgcacttttattggtctcaag |
| | (SEQ ID NO: 1) | (SEQ ID NO: 2) |
| Defb4 | cagtcatgaggatccattacctt | aatttgggtaaaggctgcaa |
| | (SEQ ID NO: 3) | (SEQ ID NO: 4) |
| Edn1 | tccttgatggacaaggagtgt | cccagtccatacggtacga |
| | (SEQ ID NO: 5) | (SEQ ID NO: 6) |
| Il6 | taccccaatttccaatgctc | tcttggtccttagccactcc |
| | (SEQ ID NO : 7) | (SEQ ID NO: 8) |
| Il12b | agcactagtttcaacaccaagaaa | cccagcctttcaaaattcttt |
| | (SEQ ID NO: 9) | (SEQ ID NO: 10) |
| Lcn2 | ccatctatgagctacaagagaacaat | tctgatccagtagcgacagc |
| | (SEQ ID NO: 11) | (SEQ ID NO: 12) |
| Nfkbiz | tatcgggtgacacagttgga | tgaatggacttccccttcag |
| | (SEQ ID NO: 13) | (SEQ ID NO: 14) |
| Rpl19 | gcatcctcatggagcacat | ctggtcagccaggagctt |
| | (SEQ ID NO: 15) | (SEQ ID NO: 16) |
| S100a7a | ccctgcaccaagagcaac | ggacccttcagggtacagg |
| | (SEQ ID NO: 17) | (SEQ ID NO: 18) |
| S100a9 | caccctgagcaagaaggaat | tgtcatttatgagggcttcattt |
| | (SEQ ID NO: 19) | (SEQ ID NO: 20) |
| Tnfa | aggctgtcgctacatcactg | acccgtagggcgattacag |
| | (SEQ ID NO: 21) | (SEQ ID NO :22) |
| U90926 | gtgattctgatggcccttct | atcttgccagggaatcttga |
| | (SEQ ID NO :23) | (SEQ ID NO :24) |

Human primers

| | | |
|---|---|---|
| ACTIN | TGTCCCCCAACTTGAGATGT | TGTGCACTTTTATTCAACTGGTC |
| | (SEQ ID NO: 25) | (SEQ ID NO: 26) |
| DEFB4 | TGATGCCTCTTCCAGGTGTT | GCCTCCTCATGGCTTTTTGC |
| | (SEQ ID NO: 27) | (SEQ ID NO: 28) |
| IL6 | ACTCACCTCTTCAGAACGAATTG | CCATCTTTGGAAGGTTCAGGTTG |
| | (SEQ ID NO: 29) | (SEQ ID NO: 30) |
| LCN2 | CCACCTCAGACCTGATCCCA | CCCCTGGAATTGGTTGTCCTG |
| | (SEQ ID NO: 31) | (SEQ ID NO: 32) |
| RPLP0 | CCTTCTCCTTTGGGCTGGTCATCCA | CAGACACTGGCAACATTGCGGACAC |
| | (SEQ ID NO: 33) | (SEQ ID NO: 34) |
| S100A7A | ACGTGATGACAAGATTGACAAGC | GCGAGGTAATTTGTGCCCTTT |
| | (SEQ ID NO: 35) | (SEQ ID NO: 36) |
| S100A9 | GGTCATAGAACACATCATGGAGG | GGCCTGGCTTATGGTGGTG |
| | (SEQ ID NO: 37) | (SEQ ID NO: 38) |
| TNFA | CCTCTCTCTAATCAGCCCTCTG | GAGGACCTGGGAGTAGATGAG |
| | (SEQ ID NO: 39) | (SEQ ID NO: 40) |

Lentiviral Transduction

Mouse Nfkbiz 3′UTR Lenti-reporter-GFP (#MT-m64048) vector or pLenti-UTR-GFR-Blank vector (#m014) were purchased from Applied Biological Materials. For lentiviral production transfection mixture of 1.5-mL of Opti-MEM medium (Invitrogen), 18 μg of psPAX2 (gift from Didier Trono, Addgene plasmid #12260), 13 μg of pCMV-V-SVG (gift from Bob Weinberg, Addgene plasmid #8454), 20 μg of lentiviral construct, and 105 μL of polyethylenimine (1 mg/mL; 25 kDa; linear form; Polysciences). The mixture was incubated at room temperature for 20 min and added to HEK-293T cells (ATCC) cultured in DMEM supplemented with 10% FBS, 2 mM L-glutamine and 100 U/mL penicillin-streptomycin in 150-cm2 tissue culture flask. 48 h later, virus-containing medium was filtered through 45 μm pore-size cellulose acetate filters and used directly for BV2 transduction in the presence of polybrene (8 μg/mL, Millipore). Next day, media was changed to fresh and two days after the cells were selected with puromycin at 5 μg/mL.

Flow Cytometry

For ROS measurements, cells were treated with DI (250 μM), loaded with 10 μM CM-H2DCFDA (Invitrogen) at RT for 30 min in Hank's balanced salt solution (HBSS). After incubation, cell were rinsed with warm HBSS, harvested and directly analyzed. Mean fluorescent intensity (MFI) of living cells is shown. For Nfkbiz 3′UTR reporter analysis GFP signal was determined in live cells. For analysis of cell viability cells were stained with propidium iodide (1 μg/mL) and percentage of negative cells was plotted. Cells were acquired on CantoII or LSRII flow cytometers (Becton Dickinson), and data were analyzed with FlowJo v.9.5.2 software (Tree Star). For gating strategy see e.g., FIG. 25.

Confocal Microscopy

BMDMs were seeded at eight-well multitest microscopy slides (MP Biomedicals). Cells were treated with DI (250 μM, 12 hours) and then stimulated with LPS (100 ng/mL, 30 min). The cells were fixed with 3% paraformaldehyde in PBS for 30 min and permeabilized with 0.1% Triton X-100 in PBS for 15 min. After washing with PBS, samples were blocked with 1% BSA for 15 min and subsequently labeled with p65-specific antibody (1:50, #8242, Cell Signaling), followed by AF568-conjugated anti-rabbit secondary antibody (1:500, #A11011, Thermo Fisher Scientific) in PBS containing 1% BSA. After labeling, the cells were washed with PBS and mounted in 50% (w/v) glycerol in PBS, pH 8.5 containing DAPI (1 μg/mL, Sigma) to label nuclei. Images of random fields of view were acquired using a Leica DMi8 confocal microscope (Leica Microsystems) equipped with a HC PL ARC 40×/1.3 oil immersion objective and exported with LAS AF Lite software (Leica).

Human Blood Monocytes Isolation and Treatment

Blood from healthy donors was acquired from LRS chambers supplied by the Mississippi Valley Regional Blood Center. Peripheral blood mononuclear cells (PBMCs) from buffy coats were recovered from the Ficoll interface after a 400 g centrifugation for 30 min. Monocytes were isolated by adherence on a cell culture dish for 1 h at 37° C. and 5% C02 in RPMI containing 1% Human Serum Albumin (Albutein, Grifols, Spain). After extensive washes to allow removal of non-adherent cells, monocytes (>95% purity) were harvested, counted and 5×105 cells were plated per well in 24-well plates. The cells were treated with DI (125 μM if not stated otherwise) for 12 h and stimulated with LPS (100 ng/mL) for 1 h or as indicated. In some experiments cells were treated with DI in presence of EtGSH (1 mM, Santa Cruz).

Primary Mouse and Human Keratinocytes

Primary keratinocytes were isolated from C57BL/6 WT newborn mice or human foreskins as previously described. 2×105 cells in 1 ml of KFSM media (Gibco cat #10725-018) [Ca2+ at 0.05 mM (mouse) and 0.09 mM (human)] were plated in 12-well tissue culture plates. After 2-3 days of cultivation, mouse or human cells were treated with DI for 12 hours and then stimulated with mouse recombinant IL-17A (100 ng/mL; cat #421-ML, R&D Systems) or human recombinant IL-17A (100 ng/mL; cat #7955-IL, R&D Systems) for 4 h or as indicated.

IMQ-Induced Psoriasis

To induce experimental psoriasis, Imiquimod (IMQ, Imiquimod Cream 5%, Perrigo. Co.) was applied daily to mice on both ears, (5 mg per ear) for 7 days. For the DI-treated mice: DI was administered via the intraperitoneal route at 20 mg/500 μL sterile PBS per mouse one day prior to IMQ application and daily thereafter for 7 days. After 7 days, mice were euthanized and ears were used for RNA extraction or histological analysis by performing H&E staining on 7 μm thick sections following paraffin embedding. Average ear thickness in each sample was quantified from images obtained at the identical settings using ImageJ. Thickness at 5 equidistant places in each image was quantified and mean of these values was used to represent results in each mouse.

SDH Activity in Mouse Heart and Liver

DI was administered to mice via the intraperitoneal route at 20 mg/500 μL sterile PBS per mouse either once per day for total length of 4 days (DI daily) or every 2 h, 3 times in total (DI overdose). In daily DI administration, last injection was 6 h before the tissue harvest, in DI overdose protocol last injection was 2 h before harvest. Mice were euthanized and heart (50 mg) and liver tissue (200 mg) were harvested, washed in PBS and processed for mitochondria isolation with Mitochondria Isolation Kit for Tissue (#89801, Thermo Scientific) according to manufacturer's instruction. For analysis of mitochondria purity cytoplasmic and mitochondrial fractions were diluted with reducing sample buffer and analyzed by western blot for SDH and GAPDH presence (see western blot analysis section). SDH activity in isolated mitochondria was analyzed using SDH Activity Colorimetric Kit (#MAK197, Sigma). Isolated mitochondria were directly resuspended in SDH Assay Buffer. In parallel, protein concentration in each sample was determined using RC/DC™ Protein Assay (Biorad) and activity was normalized to protein concentration.

Statistical Analysis

Unless stated otherwise in a specific section of the Methods, standard statistical analyses were performed using MS Excel or GraphPad Prism 7. The type and number of replicates and the statistical test used are described in the figure legends. Exact P values are shown where determined. Individual data points are shown, and the mean±s.e.m. is reported for analyses with n>2, the mean only is reported where n<2.

Data Availability

The raw and processed RNA-seq data have been deposited to Gene Expression Omnibus with access number GSE102190 and GSE110749. The original mass spectra may be downloaded from MassIVE (http://massive.ucsd.edu) using the identifier: MSV000082101. Source Data for the graphical representations found in all Figures and Extended Data Figures are provided. All other data that support the findings of this study are available from the corresponding author upon reasonable request.

### Example 3 References

1. Jha, A. K. et al. Network Integration of Parallel Metabolic and Transcriptional Data Reveals Metabolic Modules that Regulate Macrophage Polarization. Immunity 42, 419-430 (2015).
2. Lampropoulou, V. et al. Itaconate Links Inhibition of Succinate Dehydrogenase with Macrophage Metabolic Remodeling and Regulation of Inflammation. Cell Metab. 24, 158-166 (2016).
3. Cordes, T. et al. Immunoresponsive Gene 1 and Itaconate Inhibit Succinate Dehydrogenase to Modulate Intracellular Succinate Levels. J. Biol. Chem. 291, 14274-14284 (2016).
4. Gorrini, C., Harris, I. S. & Mak, T. W. Modulation of oxidative stress as an anticancer strategy. Nat. Rev. Drug Discov. 12, 931-947 (2013).
5. Kobayashi, E. H. et al. Nrf2 suppresses macrophage inflammatory response by blocking proinflammatory cytokine transcription. Nat. Commun. 7, 11624 (2016).
6. Sullivan, L. B. et al. The proto-oncometabolite fumarate binds glutathione to amplify ROS dependent signaling. Mol. Cell 51, 236-248 (2013).
7. Zheng, L. et al. Fumarate induces redox-dependent senescence by modifying glutathione metabolism. Nat. Commun. 6, 6001 (2015).
8. Medzhitov, R. & Horng, T. Transcriptional control of the inflammatory response. Nat. Rev. Immunol. 9, 692-703 (2009).
9. Yamamoto, M. et al. Regulation of Toll/IL-1-receptor-mediated gene expression by the inducible nuclear protein IκBζ. Nature 430, 218-222 (2004).
10. Dhamija, S. et al. IL-1-induced post-transcriptional mechanisms target overlapping translational silencing and destabilizing elements in IκBζ mRNA. J. Biol. Chem. 285, 29165-29178 (2010).
11. Harding, H. P. et al. An integrated stress response regulates amino acid metabolism and resistance to oxidative stress. Mol. Cell 11, 619-633 (2003).
12. Komatsu, M. et al. The selective autophagy substrate p62 activates the stress responsive transcription factor Nrf2 through inactivation of Keap1 Nat. Cell Biol. 12, 213-223 (2010).
13. Otterbein, L. E., Soares, M. P., Yamashita, K. & Bach, F. H. Heme oxygenase-1: unleashing the protective properties of heme. Trends Immunol. 24, 449-455 (2003).
14. Gilchrist, M. et al. Systems biology approaches identify ATF3 as a negative regulator of Toll-like receptor 4. Nature 441, 173-178 (2006).
15. Labzin, L. I. et al. ATF3 Is a Key Regulator of Macrophage IFN Responses. J. Immunol. 195, 4446-4455 (2015).
16. Okuma, A. et al. Enhanced Apoptosis by Disruption of the STAT3-IκB-ζ Signaling Pathway in Epithelial Cells Induces Sjögren's Syndrome-like Autoimmune Disease. Immunity 38, 450-460 (2013).
17. Muromoto, R. et al. IL-17A plays a central role in the expression of psoriasis signature genes through the induction of IκB-ζ in keratinocytes. Int. Immunol. 28, 443-452 (2016).
18. Johansen, C. et al. IκBζ is a key driver in the development of psoriasis. Proc. Natl. Acad. Sci. U.S.A. 112, 5825-5833 (2015).
19. Tsoi, L. C. et al. Enhanced meta-analysis and replication studies identify five new psoriasis susceptibility loci. Nat. Commun. 6, 7001 (2015).
20. Okada, K. et al. The α-glucosidase inhibitor acarbose prevents obesity and simple steatosis in sequestosome 1/A170/p62 deficient mice. Hepatol. Res. 39, 490-500 (2009).
21. Parada, E. et al. The microglial α7-acetylcholine nicotinic receptor is a key element in promoting neuroprotection by inducing heme oxygenase-1 via nuclear factor erythroid-2-related factor 2. Antioxid. Redox Signal. 19, 1135-1148 (2013).
22. Hartman, M. G. et al. Role for activating transcription factor 3 in stress-induced beta-cell apoptosis. Mol. Cell. Biol. 24, 5721-5732 (2004).
23. Vincent, E. E. et al. Mitochondrial Phosphoenolpyruvate Carboxykinase Regulates Metabolic Adaptation and Enables Glucose-Independent Tumor Growth. Mol. Cell 60, 195-207 (2015).
24. Sergushichev, A. An algorithm for fast preranked gene set enrichment analysis using cumulative statistic calculation. bioRxiv (2016). https://doi.org/10.1101/060012
25. Ritchie, M. E. et al. limma powers differential expression analyses for RNA-sequencing and microarray studies. Nucleic Acids Res. 43, e47-e47 (2015).
26. McGuire, V. A. et al. Dimethyl fumarate blocks pro-inflammatory cytokine production via inhibition of TLR induced M1 and K63 ubiquitin chain formation. Sci. Rep. 6, 31159 (2016).
27. Schindelin, J. et al. Fiji: an open-source platform for biological-image analysis. Nat. Methods 9, 676-682 (2012).
28. Rueden, C. T. et al. ImageJ2: ImageJ for the next generation of scientific image data. BMC Bioinformatics 18, 529 (2017).
29. Lu, W. et al. Metabolomic Analysis via Reversed-Phase Ion-Pairing Liquid Chromatography Coupled to a Stand Alone Orbitrap Mass Spectrometer. Anal. Chem. 82, 3212-3221 (2010).
30. Sahil et al. ElucidataInc/ElMaven: El-MAVEN v0.2.2. (2017). doi:10.5281/ZENODO. 1133506
31. Harder, B.-J., Bettenbrock, K. & Klamt, S. Model-based metabolic engineering enables high yield itaconic acid production by Escherichia coli. Metab. Eng. 38, 29-37 (2016).
32. Rappsilber, J., Mann, M. & Ishihama, Y. Protocol for micro-purification, enrichment, pre-fractionation and storage of peptides for proteomics using StageTips. Nat. Protoc. 2, 1896-1906 (2007).
33. Cheng, Z. et al. Pervasive, Coordinated Protein-Level Changes Driven by Transcript Isoform Switching during Meiosis. Cell 172, 910-923 (2018).
34. Keshishian, H. et al. Multiplexed, Quantitative Workflow for Sensitive Biomarker Discovery in Plasma Yields Novel Candidates for Early Myocardial Injury. Mol. Cell. Proteomics 14, 2375-2393 (2015).
35. Cox, J. J. & Mann, M. MaxQuant enables high peptide identification rates, individualized p.p.b.-range mass accuracies and proteome-wide protein quantification. Nat. Biotechnol. 26, 1367-1372 (2008).
36. Klaus Schulze-Osthoff Schmitz, et al. IκBζ IS a Transcriptional Key Regulator of CCL2/MCP-1. J. Immunol. 190, 4812-4820 (2013).
37. Stewart, S. A. et al. Lentivirus-delivered stable gene silencing by RNAi in primary cells. RNA 9, 493-501 (2003).
38. de Guzman Strong, C. et al. A milieu of regulatory elements in the epidermal differentiation complex syntenic block: implications for atopic dermatitis and psoriasis. Hum. Mol. Genet. 19, 1453-1460 (2010).
39. van der Fits, L. et al. Imiquimod-Induced Psoriasis-Like Skin Inflammation in Mice Is Mediated via the IL-23/IL-17 Axis. J. Immunol. 182, 5836-5845 (2009).

1. A method of treatment of a disease, disorder, or condition associated with an inflammatory response or an immune response comprising:
administering a therapeutically effective amount of a composition comprising itaconate, malonate, or a derivative thereof to a subject in need thereof; wherein, the therapeutically effective amount reduces or prevents inflammation or an immune response.

2. The method of claim 1, wherein the immune response is a lipopolysaccharide (LPS)-mediated immune response.

3. The method of claim 1, wherein reducing or preventing inflammation or an immune response results in reduced tissue injury during a cardiovascular infarction.

4. The method of claim 1, wherein the disease, disorder or condition is associated with Ikb-ζ or a cancer.

5. The method of claim 4, wherein administration of itaconate, malonate, or a derivative thereof inhibits tumor growth.

6. (canceled)

7. The method of claim 1, wherein the itaconate, malonate, or a derivative thereof is selected from one or more of the group consisting of: itaconate, itaconic acid, dimethyl itaconate (DI), 4-methyl itaconate, 3-(ethoxycarbonyl)but-3-enoic acid, 4-ethoxy-2-methylene-4-oxobutanoic acid, 4-octyl itaconate, dimethyl fumarate (DMF), diethyl malonate (DEM), dimethyl malonate, malonate, malonic acid, 2-methylenesuccinic acid, monoethylitaconate, 2-methyl fumaric acid, fuamaric acid, or a derivative or a pharmaceutically acceptable salt thereof, including all tautomers and stereoisomers.

8. The method of claim 1, wherein the itaconate, malonate, or a derivative thereof comprises a compound of formula I: 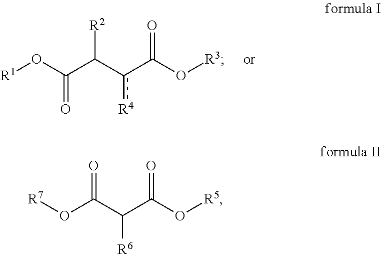
or a pharmaceutically acceptable salt thereof, including all tautomers and stereoisomers thereof wherein,
R¹ is hydrogen, unsubstituted or substituted alkyl; unsubstituted or substituted alkenes; or unsubstituted or substituted alkynes;
R² is hydrogen, unsubstituted or substituted alkyl; unsubstituted or substituted alkenes; or unsubstituted or substituted alkynes;
R³ is hydrogen, unsubstituted or substituted alkyl; unsubstituted or substituted alkenes; or unsubstituted or substituted alkynes;
R⁴ is hydrogen, unsubstituted or substituted alkyl; unsubstituted or substituted alkenes; or unsubstituted or substituted alkynes;
R⁵ is hydrogen, unsubstituted or substituted alkyl; unsubstituted or substituted alkenes; or unsubstituted or substituted alkynes;
R⁶ is hydrogen, unsubstituted or substituted alkyl; unsubstituted or substituted alkenes; or unsubstituted or substituted alkynes; and
R⁷ is hydrogen, unsubstituted or substituted alkyl; unsubstituted or substituted alkenes; or unsubstituted or substituted alkynes.

9. The method of claim 8, wherein,
R¹, R², R³, R⁴, R⁵, R⁶, or R⁷ is optionally substituted with one or more groups independently selected from the group consisting of hydroxyl; C₁₋₁₀ alkyl hydroxyl; amine; C₁₋₁₀carboxylic acid; C₁₋₁₀ carboxyl; straight chain or branched C₁₋₁₀ alkyl, optionally containing unsaturation; a C₂₋₆ cycloalkyl optionally containing unsaturation or one oxygen or nitrogen atom; straight chain or branched C₁₋₁₀ alkyl amine; C₁₋₁₀ ester; C₁₋₁₀ ether; heterocyclyl; heterocyclic amine; and aryl comprising a phenyl; heteroaryl containing from 1 to 4 N, O, or S atoms; unsubstituted phenyl ring; substituted phenyl ring; unsubstituted heterocyclyl; and substituted heterocyclyl;
the unsubstituted phenyl ring or substituted phenyl ring is optionally substituted with one or more groups independently selected from the group consisting of hydroxyl; C₁₋₁₀ alkyl hydroxyl; amine; C₁₋₁₀ carboxylic acid; C₁₋₁₀ carboxyl; straight chain or branched C₁₋₁₀ alkyl, optionally containing unsaturation; straight chain or branched C₁₋₁₀ alkyl amine, optionally containing unsaturation; a C₂₋₆ cycloalkyl optionally containing unsaturation or one oxygen or nitrogen atom; straight chain or branched C₁₋₁₀ alkyl amine; C₁₋₁₀ ester; C₁₋₁₀ ether; heterocyclyl; heterocyclic amine; aryl comprising a phenyl; and heteroaryl containing from 1 to 4 N, O, or S atoms; or
the unsubstituted heterocyclyl or substituted heterocyclyl is optionally substituted with one or more groups independently selected from the group consisting of hydroxyl; C₁₋₁₀ alkyl hydroxyl; amine; C₁₋₁₀ carboxylic acid; C₁₋₁₀ carboxyl; straight chain or branched C₁₋₁₀ alkyl, optionally containing unsaturation; straight chain or branched C₁₋₁₀ alkyl amine, optionally containing unsaturation; a C₂₋₆ cycloalkyl optionally containing unsaturation or one oxygen or nitrogen atom; C₁₋₁₀ ester; C₁₋₁₀ ether; heterocyclyl; straight chain or branched C₁₋₁₀ alkyl amine; heterocyclic amine; and aryl comprising a phenyl; and heteroaryl containing from 1 to 4 N, O, or S atoms.

10. The method of claim 9, wherein
R¹ is H, C₁₋₁₀ alkyl, or CH₃;
R² is CH₂, C₁₋₁₀ alkyl, CH₃, or H;
R³ is H, C₁₋₁₀ alkyl, CH₃, or C₈H₁₇;
R⁴ is H, C₁₋₁₀ alkyl, CH₂, or CH₃;
R⁵ is H, C₁₋₁₀ alkyl, or CH₃;
R⁶ is H, C₁₋₁₀ alkyl, or CH₃; and
R⁷ is H, C₁₋₁₀ alkyl, or CH₃.

11. The method of claim 8, wherein the compound of formula I or formula II, or a derivative thereof is selected from the group consisting of: 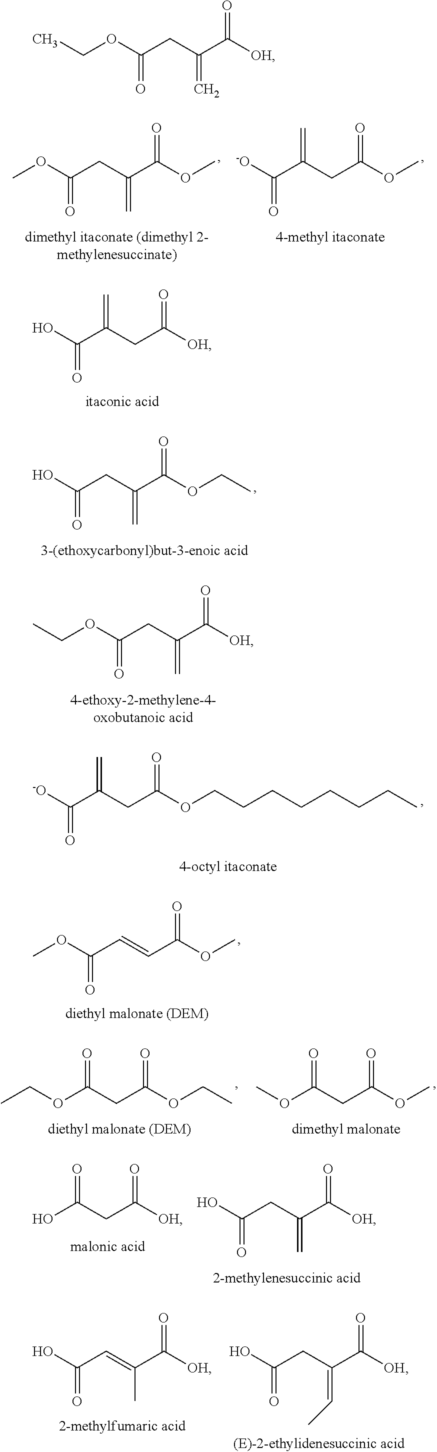 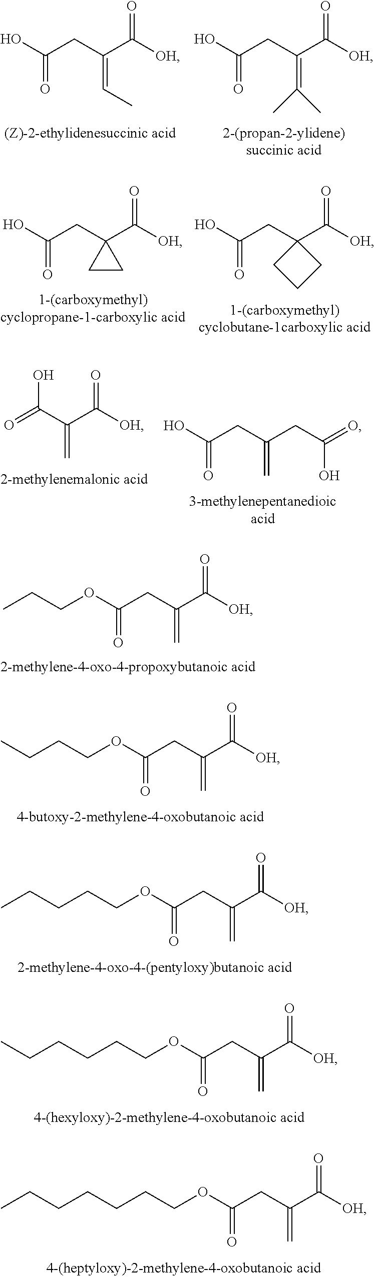 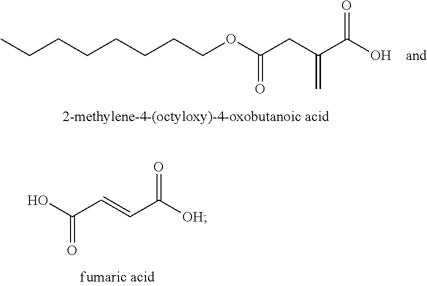 [SMILES: C=C(CC(=O)OCCCCCCCC)C(=O)O ; O=C(O)/C=C/C(=O)O]
or a pharmaceutically acceptable salt thereof, including all tautomers and stereoisomers.

12. The method of claim 1, wherein the itaconate, malonate, or a derivative thereof down-regulates Ikb-ζ induction.

13. The method of claim 12, wherein the itaconate, malonate, or a derivative thereof down-regulates Ikb-ζ induction in an Nrf2-independent manner.

14. The method of claim 13, wherein the itaconate, malonate, or a derivative thereof inhibits STAT3 activation in an Nrf2-independent manner.

15. (canceled)

16. The method of claim 1, wherein the immune response is an autoimmune response or a lipopolysaccharide (LPS)-mediated immune response; and
TNF-α production is substantially unaffected.

17. The method of claim 16, wherein the itaconate, malonate, or a derivative thereof interferes with (i) activation of pro-inflammatory macrophages, (ii) ROS-related oxidative stress, (iii) inflammatory T cell response (iv) pathogenic adaptive immune response, (v) IL-17, or (vi) GM-CSF-production; and TNF-α production is substantially unaffected.

18. The method of claim 1, wherein the disease, disorder, or condition is associated with increased expression or increased secretion of Ikb-ζ, STAT3, or IL17.

19. The method of claim 18, wherein the itaconate, malonate, or a derivative thereof inhibits: Ikb-ζ induction; STAT3 activation; IL-17-associated autoimmune inflammation; or frequency of IL-17-producing cells.

20. The method of claim 1, wherein the composition comprising itaconate, malonate, or a derivative thereof is formulated as a pharmaceutical composition comprising one or more pharmaceutically acceptable diluents or carriers.

21. The method of claim 4, wherein the Ikb-ζ associated disease, disorder, or condition is selected from the group consisting of:
(i) an NF-κB associated inflammatory diseases, such as rheumatoid arthritis (RA), atherosclerosis, multiple sclerosis, chronic inflammatory demyelinating polyradiculoneuritis, asthma, inflammatory bowel disease, Heliobacter pylori-associated gastritis, or systemic inflammatory response syndrome;
(ii) an NF-κB associated cancer, such as prostate cancer, breast cancer, lung cancer, head and neck squamous cell carcinomas, glioblastoma, skin cancer, brain cancer, glioma, liver cancer, non-small cell lung cancers (NSCLC), lymphoma, leukemia, rectal cancer, gastric cancer, or colon cancer;
(iii) a carcinoma, an autoimmune disease, Sjögren's syndrome (or a Sjögren's syndrome-like autoimmune disease), lupus, myxoid liposarcoma, brain glioblastoma multiforme, hypersensitivity syndrome, multiple sclerosis, susceptibility to pneumococcal disease, ocular surface inflammatory disorders, epilepsy, or meningococcal meningitis, atopic dermatitis, bursitis, tendinitis, psoriasis, or allergic conjunctivitis;
(iv) psoriasis, vitiligo, allergies, autoimmune disease, rheumatoid arthritis, inflammatory bowel disease, multiple sclerosis, or asthma; or
(v) an IL-17 associated cancer, such as breast cancer, lung cancer, colorectal cancer (CRC), prostate cancer, breast cancer, myeloma, melanoma, ovarian cancer, renal cell carcinoma, colon cancer, acute myeloid leukemia, gastric cancer, lymphoma, pancreatic cancer, or lung cancer.

22. (canceled)

23. The method of claim 2, wherein the Ikb-ζ modulation agent selectively down-regulates LPS-induced production of inflammatory cytokines, IL1b, IL6, or IL12, but not TNF.
